(19) United States
(12) Patent Application Publication
(10) Pub. No.: US 2023/0115515 A1
(43) Pub. Date: Apr. 13, 2023

(54) PYRAZOLYLPROPANAMIDE COMPOUNDS AND USES THEREOF FOR TREATMENT OF PROSTATE CANCER

(71) Applicant: University of Tennessee Research Foundation, Knoxville, TN (US)

(72) Inventors: Ramesh Narayanan, Cordova, TN (US); Duane D. Miller, Collierville, TN (US); Yali He, Germantown, TN (US); Thamarai Ponnusamy, Memphis, TN (US); Dong-Jin Hwang, Arlington, TN (US)

(21) Appl. No.: 17/957,539

(22) Filed: Sep. 30, 2022

Related U.S. Application Data
(63) Continuation of application No. PCT/US2021/025468, filed on Apr. 1, 2021.
(60) Provisional application No. 63/004,474, filed on Apr. 2, 2020.

(51) Int. Cl. A61K 31/415; A61K 31/4439; A61K 31/517; A61K 31/4192; A61K 31/506; A61K 31/422; A61P 35/00

(52) U.S. Cl. CPC A61K 31/415; A61K 31/4439; A61K 31/517; A61K 31/4192; A61K 31/506; A61K 31/422; A61P 35/00

(57) ABSTRACT

This invention relates to pyrazolylpropanamide compounds and uses thereof for treatment of prostate cancer, advanced prostate cancer, refractory prostate cancer, AR overexpressing prostate cancer, castration-resistant prostate cancer, castration-sensitive prostate cancer, AR-V7 expressing prostate cancer, or d567ES expressing prostate cancer, darolutamide resistant prostate cancer, enzalutamide resistant prostate cancer, apalutamide resistant prostate cancer, or abiraterone resistant prostate cancer.

### CROSS REFERENCE TO RELATED APPLICATIONS

This application is a continuation of PCT Application No. PCT/US2021/025468, filed Apr. 1, 2021, and claims the benefit of U.S. Provisional Application No. 63/004,474, filed Apr. 2, 2020, which are incorporated in their entirety herein by reference.

### FIELD OF THE INVENTION

This invention relates to pyrazolylpropanamide compounds and uses thereof for treatment of prostate cancer, advanced prostate cancer, refractory prostate cancer, AR overexpressing prostate cancer, castration-resistant prostate cancer, castration-sensitive prostate cancer, AR-V7 expressing prostate cancer, or d567ES expressing prostate cancer, darolutamide resistant prostate cancer, enzalutamide resistant prostate cancer, apalutamide resistant prostate cancer, or abiraterone resistant prostate cancer.

### BACKGROUND OF THE INVENTION

Prostate cancer (PC) is the second leading cause of cancer-related death, after lung cancer, in American men. Prostate cancer depends on the activation of androgen receptor (AR) signaling for its development, progression, growth, and survival.

Approximately 20-40% of PC patients treated with radiation and radical prostatectomy will experience tumor recurrence. Once the tumor recurs, androgen ablation therapy or androgen deprivation therapy (ADT) is the standard of care for most patients. ADT is achieved through surgical castration (orchiectomy) or chemical castration (injection of gonadotropin-releasing hormone agonist or antagonist), both of which cause a reduction in testosterone biosynthesis by testes. In addition to ADT, secondary hormonal suppression is provided by direct competitive ligand binding domain (LBD)-directed AR antagonists termed as antiandrogens such as flutamide (1), bicalutamide (2), nilutamide (3), enzalutamide (4), apalutamide (5), or darolutamide (6) or androgen synthesis inhibition such as abiraterone acetate (7) plus prednisone. Secondary hormonal suppression, that is, added to ADT, has been approved to treat castration-sensitive PC (CSPC) or castration-resistant prostate cancer (CRPC), with the approval trend toward their use earlier in the natural history of the disease in order to more effectively delay disease progression.

ADT is initially effective for advanced PCs; however, sustained ADT treatment, in combination with antiandrogens, often only stabilizes the disease for 2-3 years before PC becomes refractory, resulting in a more aggressive CRPC tumor phenotype where tumors become resistant to (ongoing ADT and) secondary hormonal therapies. Resistance to any one of bicalutamide (2), enzalutamide (4), apalutamide (5), or abiraterone acetate (7) can emerge just months after initiation and studies suggest that darolutamide (6) may behave similarly in the CPRC population (darolutamide (6) approved for mCSPC). Despite resistance to secondary hormonal therapies in CRPC whether direct (1-6) or indirect (7), AR signaling continues to be fundamental for tumor growth and disease progression. Correspondingly, novel mechanisms to inhibit the AR axis are needed in hormone-resistant PCs.

Although the exact mechanisms of CRPC progression are not always known clinically nor are they mutually exclusive, preclinical and clinical research has demonstrated numerous contributing factors to the emergence of CRPC that include (i) compensatory production of intratumoral androgens (e.g., DHT synthesized from adrenal precursors), (ii) AR gene amplifications and overexpression, (iii) AR LBD point mutations, (iv) alterations in the expression of coregulatory proteins, (v) ligand-independent activation of AR, (vi) constitutively active truncated AR splice variants (AR SVs), and (vii) induction of intracrine androgen metabolic enzymes. Direct and indirect antiandrogen therapies all target AR at the LBD and eventually fail because of the resistance mechanisms mentioned above. The development of AR antagonists for CRPC with novel mechanisms of action that are capable of durably treating patients with resistance to bicalutamide (2), enzalutamide (4), apalutamide (5), and abiraterone acetate (7) (cross-resistance of 7 to 4 and 5 is common; flutamide (1) and nilutamide (3) are rarely used) or darolutamide (6) (approved in 2019; patterns of resistance to 6 are still emerging) is an urgent need.

To provide clinical benefit for CRPC or to circumvent the emergence of CRPC, the next generation of AR-targeted therapeutics ideally should be able to bind to novel and/or multiple domains of the AR and inhibit a broad scope of AR functions across the broad scope of AR sequences present and emerging in the heavily pretreated CPRC population. Such novel antagonists ideally will maintain activity in wild-type (wt), point mutant, AR SVs, and/or AR overexpressing pathogenic states with sufficient potency to maintain suppression of the AR axis as PC becomes progressively more refractory to treatment.

Binding to a non-LBD site and degradation of the AR protein are promising preclinical approaches to rationally target CRPC. Degradation of AR can be achieved by genetic knockdown technologies, such as antisense oligonucleotides, RNA interference, and DNA editing. Despite genetic approaches having great therapeutic potential, it remains clinically challenging due to technical difficulties in delivering oligonucleotides (polyanionic macromolecules) to the prostate and metastatic tumors. Further, oligonucleotide uptake into the tumor cells is poor. Alternatively, targeted destruction of the AR by protein knockdown technologies which degrade AR via the ubiquitin proteasome system (UPS) remain promising options yet to be tested definitively in the clinical setting.

In recent antitumor studies, the discovery and characterization of AR antagonists have been reported that selectively inhibit tumor growth and degrade the AR (full-length) and AR SVs (truncated) within these tumors (Ponnusamy, et al. Cancer Res. 2017, 77, 6282-6298). A series of aryl indol-1-yl propanamides and aryl indolin-1-yl propanamides has been reported as selective androgen receptor degraders (SARDs) (Hwang, et al. J. Med. Chem. 2019, 62, 491-511). These SARD activities were mediated through the UPS as determined by UPS inhibitor studies (Ponnusamy, et al. Cancer Res. 2017, 77, 6282-6298; Ponnusamy, et al. Clin. Cancer Res. 2019, 25, 6764-6780). The SARDs have been found to degrade AR and inhibit AR function and exhibit in vitro inhibitory potency in screening assays (e.g., LBD binding, transcriptional inhibition, AR degradation, and antiproliferative assays) and greater in vivo efficacy (Hershberger assay and various AR-dependent CPRC xenografts) than the approved AR antagonists.

Pyrazolylpropanamide compounds as described herein are selective androgen receptor degraders (SARDs) and pan-antagonists. These compounds exhibit potent AR antagonist activities, including promising distribution, metabolism, and pharmacokinetic properties, and broad-spectrum AR antagonist properties, including potent in vivo antitumor activity.

### SUMMARY OF THE INVENTION

In one aspect, the present invention provides a method of treating prostate cancer in a subject in need thereof, comprising administering to the subject a therapeutically effective amount of a compound represented by the structure of formula I

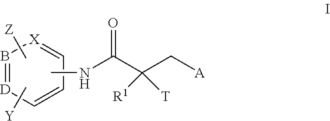

wherein
T is OH;
R¹ is CH₃;
Y is H, CF₃, F, I, Br, Cl, or CN;
Z is H, NO₂, CN, halogen, COOR, COR, NHCOR, or CONHR;
or Y and Z form a 5 to 8 membered fused ring;
X and D are each CH or N;
B is a bond or CH, and when B is a bond, D=B-X is represented by D-X;
R is H, alkyl, haloalkyl, alkyl-OH, aryl, F, Cl, Br, I, or OH;
A is a five-membered unsaturated ring having at least one nitrogen atom and 0, 1, or 2 double bonds, optionally substituted with at least one of Q¹, Q², Q³ and Q⁴, each independently selected from linear or branched alkyl, haloalkyl, CF₃, aryl, F, Cl, Br, I, CN, NO₂, OR, benzyl, alkynyl, SO₂N(R)₂, NH COOR, N(R)₂, NHCOR, CONHR, COOR, or COR; wherein said alkyl, alkynyl, and aryl are each optionally substituted with halogen, CN, or OH, or its optical isomer, pharmaceutically acceptable salt, hydrate or any combination thereof.

In some embodiments, the prostate cancer is advanced prostate cancer, refractory prostate cancer, AR overexpressing prostate cancer, castration-resistant prostate cancer, castration-sensitive prostate cancer, AR-V7 expressing prostate cancer, or d567ES expressing prostate cancer.

In some embodiments, the castration resistant prostate cancer (CRPC) is metastatic CRPC (mCRPC), non-metastatic CRPC (nmCRPC), or high-risk nmCRPC.

In some embodiments, the castration-resistant prostate cancer is AR overexpressing castration-resistant prostate cancer, F876L mutation expressing castration-resistant prostate cancer, F876L_T877 A double mutation expressing castration-resistant prostate cancer, AR-V7 expressing castration-resistant prostate cancer, d567ES expressing castration-resistant prostate cancer, and/or castration-resistant prostate cancer characterized by intratumoral androgen synthesis.

In some embodiments, the castration-sensitive prostate cancer is F876L mutation expressing castration-sensitive prostate cancer, F876L_T877 A double mutation castration-sensitive prostate cancer, and/or castration-sensitive prostate cancer characterized by intratumoral androgen synthesis. In some embodiments, the treating of castration-sensitive prostate cancer is conducted in a non-castrate setting, or as monotherapy, or when castration-sensitive prostate cancer tumor is resistance to enzalutamide, apalutamide, and/or abiraterone.

In some embodiments, the method of the invention further comprises administering androgen deprivation therapy (ADT).

In some embodiments, the prostate cancer is resistant to treatment with an androgen receptor antagonist. In some embodiments, the androgen receptor antagonist is at least one of darolutamide, enzalutamide, apalutamide, bicalutamide, abiraterone, EPI-001, EPI-506, AZD-3514, galeterone, ASC-J9, flutamide, hydroxyflutamide, nilutamide, cyproterone acetate, ketoconazole, or spironolactone.

In some embodiments, the prostate cancer is darolutamide resistant prostate cancer, enzalutamide resistant prostate cancer, apalutamide resistant prostate cancer, or abiraterone resistant prostate cancer. In some embodiments, the prostate cancer is darolutamide resistant prostate cancer. In some embodiments, the prostate cancer is enzalutamide resistant prostate cancer. In other embodiments, the prostate cancer is apalutamide resistant prostate cancer. In some embodiments, the prostate cancer is abiraterone resistant prostate cancer.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. 1 depicts antagonism of F876L-mutant AR transactivation. AR with phenylalanine 876 mutated to leucine (F876L), GRE-LUC, and CMV-renilla LUC were transfected in COS cells. Cells were treated 24 h after transfection with 0.1 nM R1881 (agonist) and a dose response of antagonists. Luciferase assay was performed 48 h after transfection. The effect of each compound was conducted in antagonistic mode (in the presence of 0.1 nM R1881).

FIG. 2 depicts antagonism of wtPR transactivation. COS cells were transfected with wtPR and a transactivation study was performed as in FIG. 1.

FIG. 3 depicts that SARDs antagonized AR function in prostate cancer cell, LNCaP. LNCaP cells were maintained for 2 din charcoal-stripped, serum containing medium. The cells were treated with antagonist as indicated in the figure for 20-24 h, RNA was isolated, and expression of AR-target gene, FKBP5, was measured and normalized to GAPDH using real-time PCR.

FIG. 4 depicts enzalutamide resistant LNCaP (MR49F) cellular antiproliferation. Enzalutamide (4) resistant (Enz-R) LNCaP (MR49F) cells were plated in 1% charcoal-stripped, serum-containing medium and treated with 0.1 nM R1881 and a titration of antagonist as indicated in the figure. Cells were retreated 3 d after the first treatment and the number of viable cells measured by Cell-Titer Glo assay (Promega, Madison, Wis.). N=3.

FIG. 5 depicts that SARDs degraded enzalutamide resistance conferring escape mutant AR. Enzalutamide (4) resistant (Enz-R) LNCaP cells (MR49F) (top panel) or 22RV1 cells (bottom panel) were maintained in charcoal-stripped, serum containing medium for 2 d and treated with 0.1 nM R1 881 (agonist) and a titration of the SARD or enzalutamide as indicated in the figure. Twenty-four hours after treatment, cells were harvested, protein extracted, and the protein were blotted with AR-N20 antibody. Blots were stripped and re-probed with a GAPDH antibody. The ratio of AR to GAPDH or each lane is given under each blot.

FIG. 6 depicts concentration-time plots in rats for compound 26a. Twelve week old male Sprague Dawley rats were dosed in five groups of five animals each (N=5) at the doses shown. Blood samples were drawn at the shown time points and analyte concentrations were determined by MS/MS. The concentration-time plots for all dose groups are shown for Day 1 (left) and Day 7 (right).

FIGS. 7A and 7B depict that SARDs and pan-antagonists inhibited androgen-dependent organs in rats. FIGS. 7 A and 7B show the reduction of VP and SV weights following the treatment of intact rats with 20 mg/kg (mpk) po daily of antagonist or vehicle for 14 days (n=5/group). Rats were sacrificed at the end of the treatment period and weights of prostate and SVs were measured and normalized to body weight.

FIGS. 8A and 8B depict that SARDs and pan-antagonists inhibited growth of enzalutamide-resistant prostate cancer. Enzalutamide-resistant MDVR cells (10×10⁶ cells/rat) were implanted subcutaneously in male SRG (Sprague Dawley-Rag2: IL2rg KO) rats. When the tumors reached 1000-3000 mm³, the animals were randomized and treated (intact). Once the tumors attain 2000-3000 mm³, the animals were treated orally with vehicle (DMSO/PEG-300 15:85) or 10 mg/kg/day of 26a. Tumor volume (T.V.) was measured twice weekly and represented as percent change (FIG. 9A) or weight at sacrifice (FIG. 8B).

FIG. 9 depicts PK results in rats for 21a. Sprague Dawley rats were dosed with 30 mg/kg 21a and blood was collected from jugular vein at 5 min, 30 min, 1 h, 3 h, 360 h, 12 h, and 24 h post dosing. Serum was separated and analyzed using LC/MS-MS for the amount of 21a.

FIG. 10 depicts that the pyrazolylpropanamide compounds except 29q were AR antagonists and they all efficiently antagonized the AR activity induced by androgen R1881. COS-7 cells were plated in 24 well plates in DME+5% csFBS w/o at 30,000 cells/well. Cells were transfected with 0.25 μg GRE-LUC, 10 ng CMV-renilla-LUC, and 25 ng human AR plasmids in lipofectamine transfection reagent. Cells were treated 24 hours after transfection and luciferase assay was performed 24 hours after treatment. Firefly luciferase values were normalized to renilla luciferase.

FIGS. 11A and 11B depict that compound 21c inhibited AR and AR-V7-positive 22RV1 xenograft by 63%, while enzalutamide failed to inhibit the growth. While the tumor volume of vehicle-treated animals increased from 315 to 2300 mm³, the volume of 2 lc-treated animals increased from 301 to 1205 mm³. Maximum TV inhibition in 505 arm was 63%. Animals in vehicle and 505-treated groups that have not attained euthanasia criteria will continue until they reach euthanasia. 22RV1 cells (2 million/mouse) were implanted subcutaneously in NSG mice. Once the tumors grow to 100-400 mm3 volume (length*width*width), the animals were randomized based on tumor volume and treated orally. Tumor volume was measured twice weekly. Animals were sacrificed at the end of study and tumors were collected for further analysis.

FIG. 12 depicts that Kaplan-Meier graph was plotted for animals bearing 22RV1 tumors (shown in FIGS. 11A and 11B). Euthanasia criteria is when the tumors reach>2 cm or a volume of 2000 mm³. Tumor-bearing animals that were treated with vehicle and enzalutamide reached euthanasia criteria earlier than the animals treated with 21c. Euthanasia criteria (length>2 cm or volume>2000 mm³). Kaplan-Meier plot for euthanasia criteria was created to show the difference in survival.

FIGS. 13A-13C depict that compound 21c and 10 significantly inhibited the growth triple-negative breast cancer (TNBC) patient-derived xenograft (PDX) UT-1355. UT-1355 expresses both AR and AR-V7. While vehicle-treated tumors grew from 237 to 1355, 21c and 10-treated tumors increased from 227 and 427 to 331 and 1354, respectively. These results show that 10 and 21c are effective against the PDX. TNBC breast cancer specimen from a patient was implanted in mice. Once the tumors grew, the P-1 tumors were frozen. The P1 tumors were implanted in 60 female NSG mice in August 2020. Once the tumors grew to 100-400 mm³, the animals were randomized and treated with vehicle (n=12), 60 mpk 21c (n=11), 10 (n=9; 60 mpk b.i.d) and enzalutamide (n=9; 60 mpk) groups. Body weight was measured on the day of treatment initiation and end of the study. Tumor volume was measured twice weekly. Characterization: The PDX sample was evaluated for AR expression. AR band at 110 Kda was observed. However, another band about 70 Kda was also observed. Western blot with AR-V7 antibody showed a band at 70 Kda. Patient and tumor characteristics: African American, Age 65, Collection date 01/02/2020, ki67 45-50%

FIG. 14 depicts the p-value of the tumor volume from FIGS. 13A-13C.

FIG. 15 depicts that Kaplan-Meier graph was plotted for animals bearing UT-1355 tumors (shown in FIGS. 13A-13C). Euthanasia criteria is when the tumors reach>2 cm or a volume of 2000 mm³. Tumor-bearing animals that were treated with vehicle and enzalutamide reached euthanasia criteria earlier than the animals treated with 21c. Euthanasia criteria (length>2 cm or volume>2000 mm³).

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Androgens act in cells by binding to the AR, a member of the steroid receptor superfamily of transcription factors. As the growth and maintenance of prostate cancer (PCa) is largely controlled by circulating androgens, treatment of PCa heavily relies on therapies that target AR. Treatment with AR antagonists such as darolutamide, enzalutamide, abiraterone (an indirect AR antagonist; others are LBD binding direct AR antagonists), apalutamide, bicalutamide or hydroxyflutamide to disrupt receptor activation has been successfully used in the past to reduce PCa growth. All currently available direct AR antagonists competitively bind AR and recruit corepressors such as NCoR and SMRT to repress transcription of target genes. However, altered intracellular signaling, AR mutations, and increased expression of coactivators lead to functional impairment of antagonists or even transformation of antagonists into agonists.

Studies have demonstrated that mutation of W741 and T877 within AR converts bicalutamide and hydroxyflutamide, respectively, to agonists. Similarly, increased intracellular cytokines recruit coactivators instead of corepressors to AR-responsive promoters subsequently converting bicalutamide to an agonist. Mutations that have been linked to enzalutamide, apalutamide, and abiraterone resistance include F876, H874, T877, and di-mutants T877/5888, T877/D890, F876/T877 (i.e., MR49 cells), and H874/T877 (Genome Biol. (2016) 17:10 (doi: 10.1186/s13059-015-0864-1)).

Abiraterone resistance mutations include L702H mutations which results in activation of the AR by glucocorticoids such as prednisone, causing resistance to abiraterone because abiraterone is usually prescribed in combination with prednisone. If resistance develops to enzalutamide or apalutamide then often the patient is refractory to abiraterone also and vice versa; or the duration of response is very short.

Darolutamide also has limited efficacy and duration of action in CRPC. This situation highlights the need for a definitive androgen ablation therapy to prevent AR reactivation in advanced prostate cancers. Arora et al. in Cell 155, 1309-1322 reported the induction of glucocorticoid receptor (GR) expression as a common feature of drug-resistant tumors from prostate cancer cell lines (LNCaP/AR) and clinical samples. GR substituted for the AR to activate a similar but distinguishable set of target genes and was necessary for maintenance of the resistant phenotype. The GR agonist dexamethasone was sufficient to confer enzalutamide (or apalutamide) resistance, whereas a GR antagonist restored sensitivity. Acute AR inhibition resulted in GR upregulation in a subset of prostate cancer cells due to relief of AR-mediated feedback repression of GR expression. These findings establish a mechanism of escape from AR blockade through expansion of cells primed to drive AR target genes via an alternative nuclear receptor upon drug exposure. The pyrazolylpropanamide compounds as described herein may be potent GR antagonists in addition to potent AR antagonists. As such, they would possibly prevent the emergence of GR-dependent antiandrogen resistance or treat antiandrogen resistant prostate cancers which are dependent on GR. Though specific AR mutations or AR bypass mechanisms for conferring resistance to darolutamide have not yet been reported, darolutamide binds to the same LBD target on AR and resistance mutations are likely to develop that will be sensitive to the pyrazolylpropanamide compounds as described herein.

The present invention relates to pyrazolylpropanamide compounds, which are selective androgen receptor degraders (SARDs) and pan-antagonists. The pyrazolylpropanamide compounds as described herein can be used for treatment of prostate cancer, advanced prostate cancer, refractory prostate cancer, AR overexpressing prostate cancer, castration-resistant prostate cancer, castration-sensitive prostate cancer, AR-V7 expressing prostate cancer, or d567ES expressing prostate cancer, darolutamide resistant prostate cancer, enzalutamide resistant prostate cancer, apalutamide resistant prostate cancer, or abiraterone resistant prostate cancer.

As used herein, unless otherwise defined, a “selective androgen receptor degrader” (SARD) compound is an androgen receptor antagonist capable of inhibiting the growth of PCa cells and tumors that are dependent on AR-full length (AR-FL) and/or AR splice variants (ARSV) for proliferation. The SARD compound may not bind to ligand binding domain (LBD). Alternatively, a “selective androgen receptor degrader” (SARD) compound is an androgen receptor antagonist capable of causing degradation of a variety of pathogenic mutant variant AR's and wildtype AR and hence are capable of exerting anti-androgenism is a wide variety of pathogenic altered cellular environments found in the disease states embodied in this invention. In one embodiment, the SARD is orally active. In another embodiment, the SARD is applied topically to the site of action.

The SARD compound may bind to the N-terminal domain (NTD) of the AR; to an alternate binding and degradation domain (BDD) of the AR; to both the AR ligand binding domain (LBD) and to an alternate binding and degradation domain (BDD); or to both the Nterminal domain (NTD) and to the ligand binding domain (LBD) of the AR. In one embodiment, the BDD may be located in the NTD. In one embodiment, the BDD is located in the AF-1 region of the NTD. Alternatively, the SARD compound may be capable of: inhibiting growth driven by the N-terminal domain (NTD)-dependent constitutively active AR-SV; or inhibiting the AR through binding to a domain that is distinct from the AR LBD. Also, the SARD compound may be a strong (i.e., highly potent and highly efficacious) selective androgen receptor antagonist, which antagonizes the AR stronger than other known AR antagonists (e.g., darolutamide, enzalutamide, apalutamide, bicalutamide and abiraterone).

The SARD compound may be a selective androgen receptor antagonist, which targets AR-SVs, which cannot be inhibited by conventional antagonists. The SARD compound may exhibit any one of several activities including, but not limited to: AR-SV degradation activity; AR-FL degradation activity; AR-SV inhibitory activity (i.e., is an AR-SV antagonist); AR-FL inhibitory activity (i.e., is an AR-FL antagonist); inhibition of the constitutive activation of ARSVs; or inhibition of the constitutive activation of AR-FLs. Alternatively, the SARD compound may possess dual AR-SV degradation and AR-SV inhibitory functions, and/or dual AR-FL degradation and AR-FL inhibitory functions; or alternatively possess all four of these activities.

The SARD compound may also degrade AR-FL and AR-SV. The SARD compound may degrade the AR through binding to a domain that is distinct from the AR LBD. The SARD compound may possess dual degradation and AR-SV inhibitory functions that are distinct from any available CRPC therapeutics. The SARD compound may inhibit the re-activation of the AR by alternate mechanisms such as: intracrine androgen synthesis, expression of AR-SV that lack ligand binding domain (LBD) and AR-LBD mutations with potential to resist antagonists or inhibit re-activated androgen receptors present in pathogenic altered cellular environments.

Examples of AR-splice variants include, but are not limited to, AR-V7 and ARv567es (a.k.a. AR-V12; S. Sun, et al. Castration resistance in human prostate cancer is conferred by a frequently occurring androgen receptor splice variant. J Clin Invest. (2010) 120(8), 2715-2730). Nonlimiting examples of AR mutations conferring antiandrogen resistance are: W741L, T877 A, and F876L (J. D. Joseph et al. A clinically relevant androgen receptor mutation confers resistance to second-generation antiandrogens enzalutamide and ARN-509 [apalutamide]. Cancer Discov. (2013) 3(9), 1020-1029) mutations. Many other LBD resistance conferring mutations are known in the art and will continue to be discovered. AR-V7 is a splice variant of AR that lacks the LBD (A. H. Bryce & E. S. Antonarakis. Androgen receptor splice variant 7 in castration-resistant prostate cancer: Clinical considerations. Int J Ural. (2016 Jun. 3) 23(8), 646-53. doi: 10.1111/iju.13134). It is constitutively active and has been demonstrated to be responsible for aggressive PCa and resistance to endocrine therapy.

As used herein, in some embodiments, the term “pan-antagonist” refers to antagonists that are effective against wildtype AR and all AR mutants as tested, including, but not limited to, F876L, T877A, and W741L.

As used herein, “UT-1355” is a triple-negative breast cancer (TNBC) patient-derived xenograft (PDX) developed by the inventors of the application. It is a TNBC patient specimen that grew in animals as tumors.

The pyrazolylpropanamide compounds as described herein are SARDs and panantagonists, which can be used in treating CRPC that cannot be treated with any other antagonist. The pyrazolylpropanamide compounds may treat CRPC by degrading AR-SVs. The pyrazolylpropanamide compounds may maintain their antagonistic activity in AR mutants that normally convert AR antagonists to agonists. For instance, the pyrazolylpropanamide compounds maintain their antagonistic activity to AR mutants W741L, T877 A, and F876L (J. D. Joseph et al. A clinically relevant androgen receptor mutation confers resistance to second-generation antiandrogens enzalutamide and ARN-509 [apalutamide]. Cancer Discov. (2013) 3(9), 1020-1029). Alternatively, the pyrazolylpropanamide compounds elicit antagonistic activity within an altered cellular environment in which LED-targeted agents are not effective or m which NTD-dependent AR activity is constitutively active. Alternatively, pyrazolylpropanamide compounds can be co-antagonists of AR and GR and thereby overcome or prevent antiandrogen resistant CRPC in which GR is overexpressed and/or GR is activating the AR axis. Alternatively, pyrazolylpropanamide compounds are co-antagonists of AR and PR and thereby overcome or prevent antiandrogen resistant CRPC in which PR is overexpressed and/or PR is activating the AR axis.

The present invention provides a method of treating prostate cancer in a subject in need thereof, comprising administering to the subject a therapeutically effective amount of a compound represented by the structure of formula I

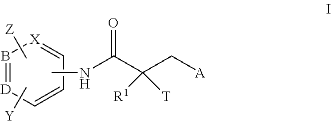

wherein
T is OH;
R¹ is CH₃;
Y is H, CF₃, F, I, Br, Cl, or CN;
Z is H, NO₂, CN, halogen, COOR, COR, NHCOR, or CONHR;
or Y and Z form a 5 to 8 membered fused ring;
X and D are each CH or N;
B is a bond or CH, and when B is a bond, D=B-X is represented by D-X;
R is H, alkyl, haloalkyl, alkyl-OH, aryl, F, Cl, Br, I, or OH;
A is a five-membered unsaturated ring having at least one nitrogen atom and 0, 1, or 2 double bonds, optionally substituted with at least one of Q¹, Q², Q³ and Q⁴, each independently selected from linear or branched alkyl, haloalkyl, CF₃, aryl, F, Cl, Br, I, CN, NO₂, OR, benzyl, alkynyl, SO₂N(R)₂, NH COOR, N(R)₂, NHCOR, CONHR, COOR, or COR; wherein said alkyl, alkynyl, and aryl are each optionally substituted with halogen, CN, or OH, or its optical isomer, pharmaceutically acceptable salt, hydrate or any combination thereof.

In some embodiments, the compound is represented by a compound of formula II

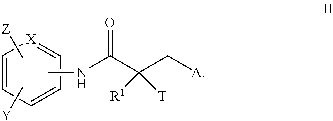

In some embodiments, the compound is represented by a compound of formula IIA or formula IIB:

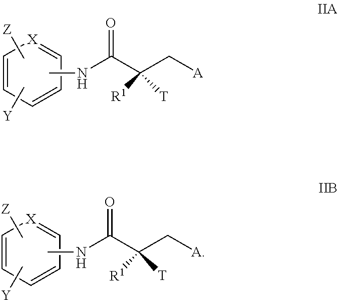

In some embodiments, the compound is represented by a compound of formula III:

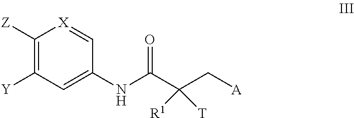

wherein
T is OH;
R¹ is CH₃;
Y is H, CF₃, F, I, Br, Cl, or CN;
Z is H, NO, CN, halogen, COOR, COR, NHCOR, or CONHR;
or Y and Z form a 5 to 8 membered fused ring;
X is CH or N;
R is H, alkyl, haloalkyl, alkyl-OH, CF₃, CH₂Cl, CH₂CH₂Cl, aryl, F, Cl, Br, I, or OH;
A is a pyrrole, pyrazole, triazole, or imidazole, each optionally substituted with at least one of Q¹, Q², Q³ and Q⁴, each independently selected from linear or branched alkyl, haloalkyl, CF₃, aryl, F, Cl, Br, I, CN, NO₂, OR, benzyl, alkynyl, SO₂N(R)₂, NHCOOR, N(R)₂, NHCOR, CONHR, COOR, or COR; wherein said alkyl, alkynyl, and aryl are each optionally substituted with halogen, CN, or OH, or its optical isomer, pharmaceutically acceptable salt, hydrate or any combination thereof.

In some embodiments, the compound is represented by the structure of formula IIIA or formula IIIB:

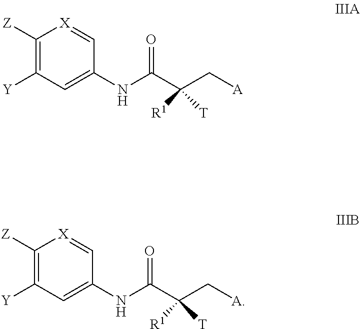

In some embodiments, the compound is represented by the structure of formula IV:

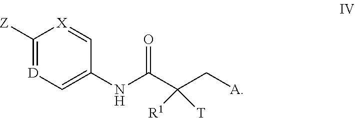

In some embodiments, the compound is represented by the structure of formula IVA or formula IVB:

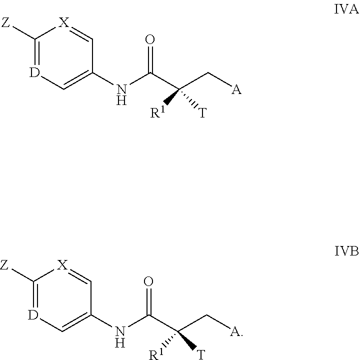

In some embodiments, the compound is represented by the structure of formula V:

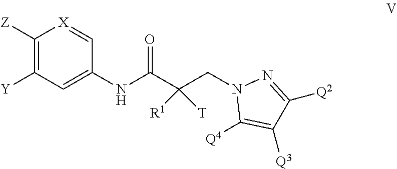

wherein
Q², Q³ and Q⁴ are each independently selected from linear or branched alkyl, haloalkyl, CF₃, aryl, F, Cl, Br, I, CN, NO₂, OR, benzyl, alkynyl, SO₂N(R)₂, NHCOOR, N(R)₂, NHCOR, CONHR, COOR, or COR; wherein said alkyl, alkynyl, and aryl are each optionally substituted with halogen, CN, or OH,
or its optical isomer, pharmaceutically acceptable salt, hydrate or any combination thereof.

In some embodiments, the compound is represented by the structure of formula VA or formula VA:

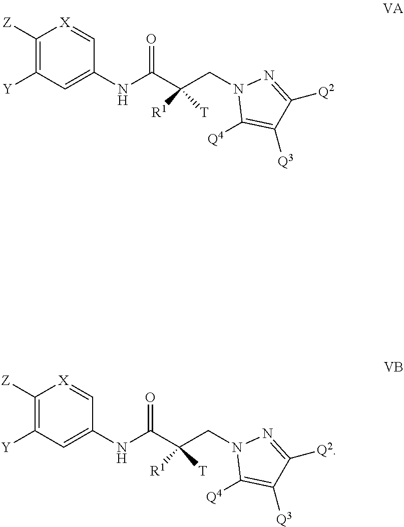

In some embodiments, Q¹, Q², Q³ and Q⁴ is CN, NO₂, CF₃, F, Cl, Br, I, alkynyl, SO₂N(R)₂, NHCOOR, N(R)₂, NHCOR, COR, or phenyl, wherein said phenyl is optionally substituted with halogen, CN, or OH.

In some embodiments, the compound is represented by any one of the following compounds:

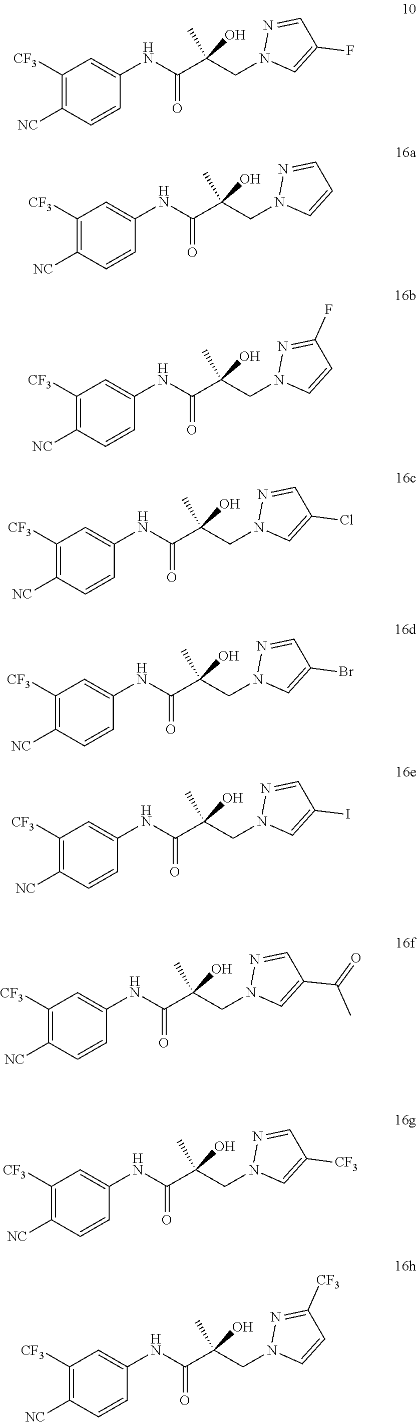

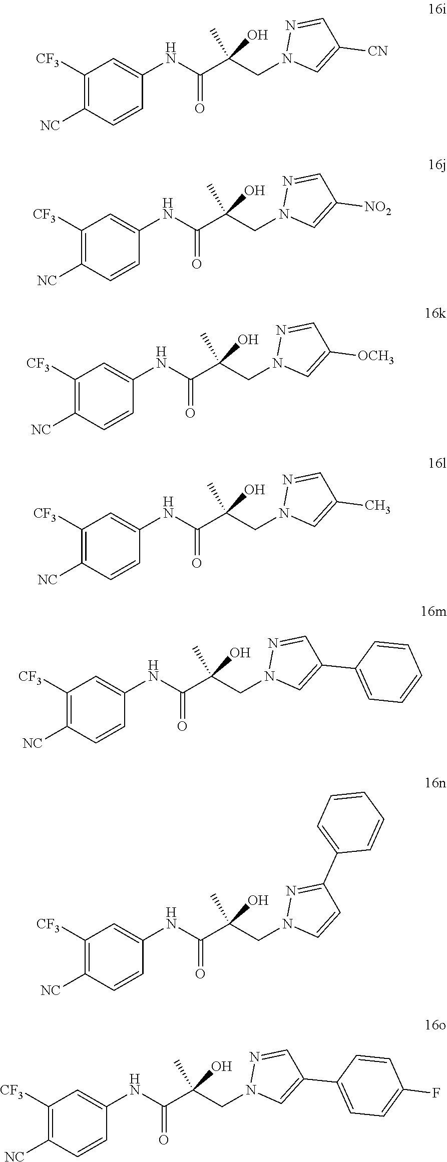

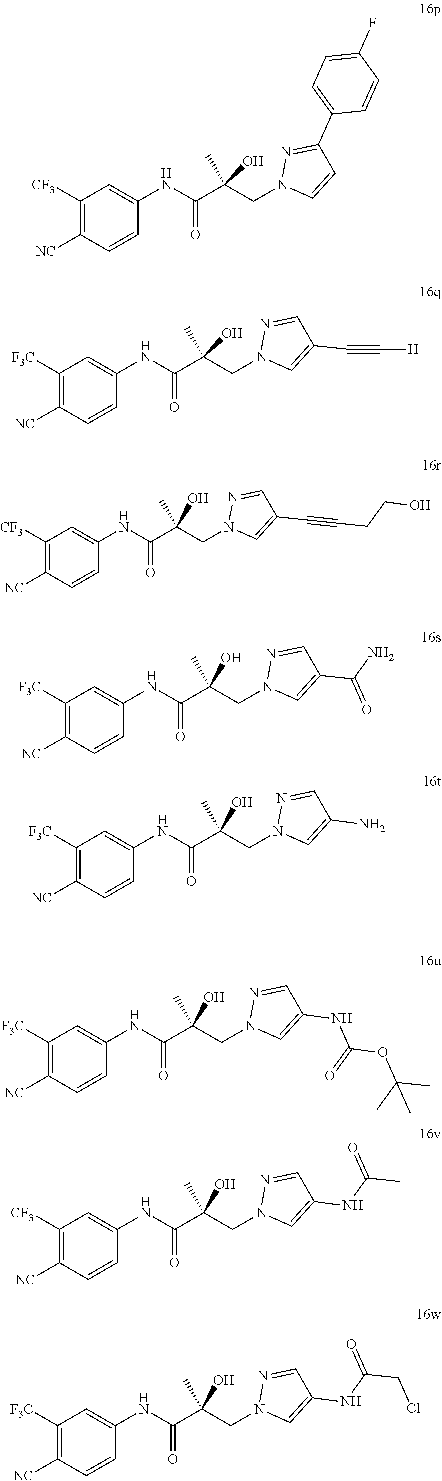

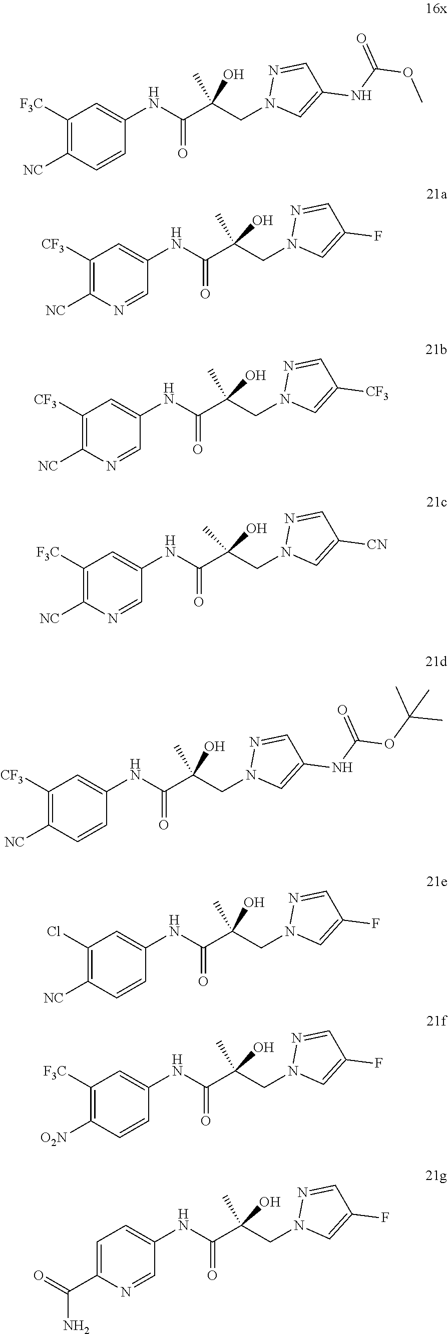

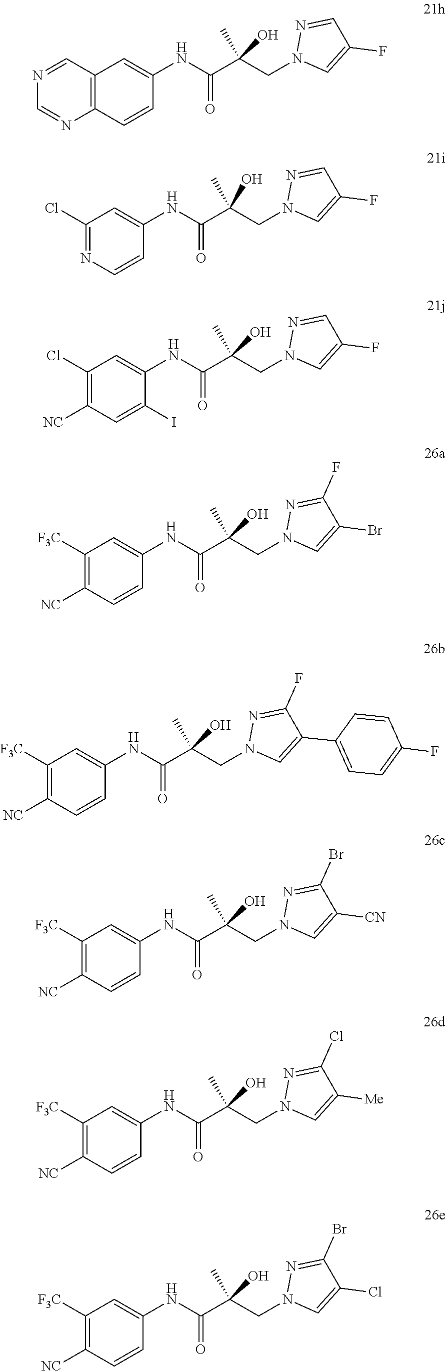

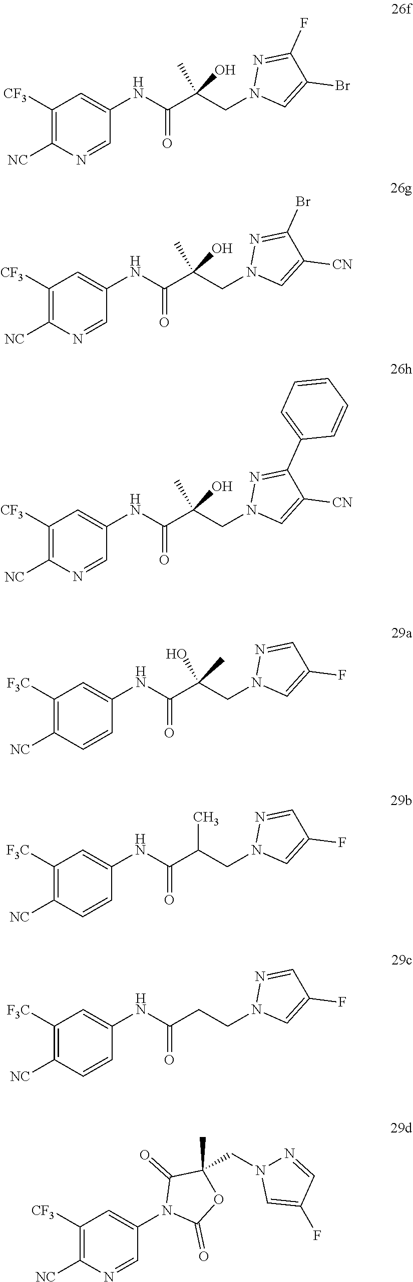

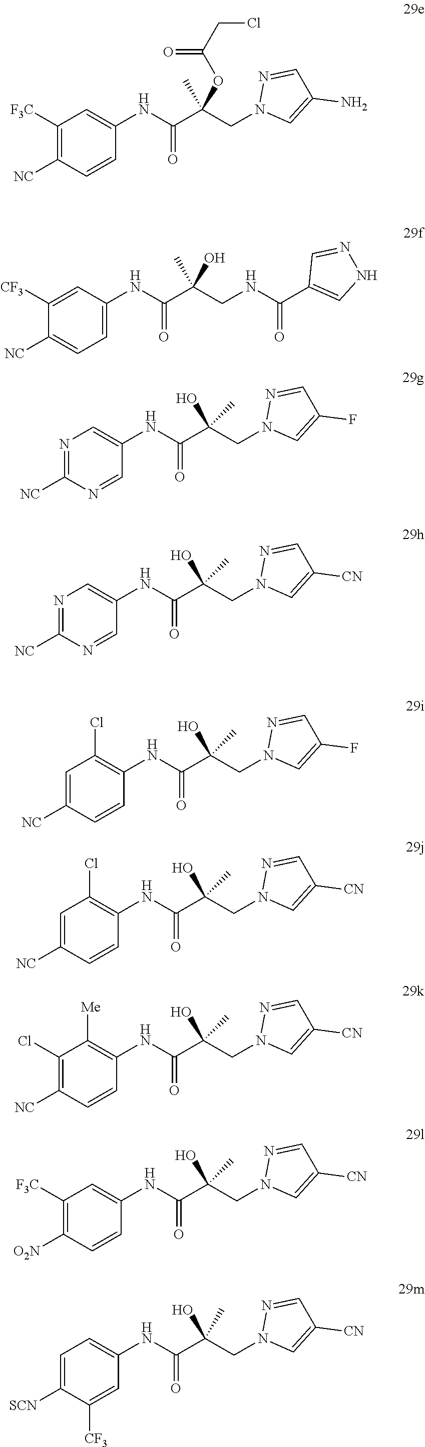

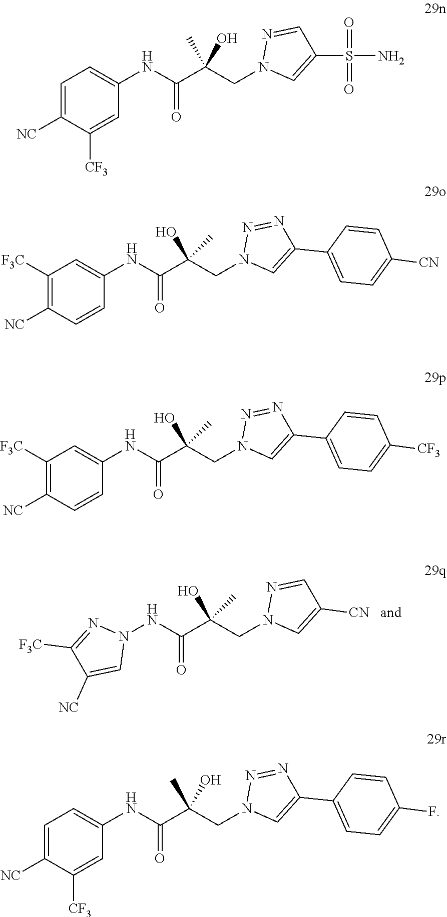 [SMILES: C[C@](O)(Cn1cc(S(N)(=O)=O)cn1)C(=O)Nc1ccc(C#N)c(C(F)(F)F)c1 ; C[C@](O)(Cn1cc(-c2ccc(C#N)cc2)nn1)C(=O)Nc1ccc(C#N)c(C(F)(F)F)c1 ; C[C@](O)(Cn1cc(-c2ccc(C(F)(F)F)cc2)nn1)C(=O)Nc1ccc(C#N)c(C(F)(F)F)c1 ; C[C@](O)(Cn1cc(C#N)cn1)C(=O)Nn1cc(C#N)c(C(F)(F)F)n1 ; "and" ; C[C@@](O)(Cn1cc(-c2ccc(F)cc2)nn1)C(=O)Nc1ccc(C#N)c(C(F)(F)F)c1]

In some embodiments, the compound is represented by compound 26a

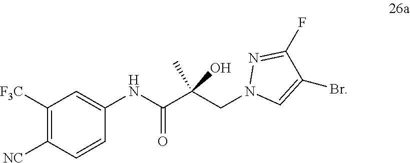 [SMILES: C[C@](O)(Cn1cc(Br)c(F)n1)C(=O)Nc1ccc(C#N)c(C(F)(F)F)c1]

In some embodiments, the compound is represented by any one of the following compounds

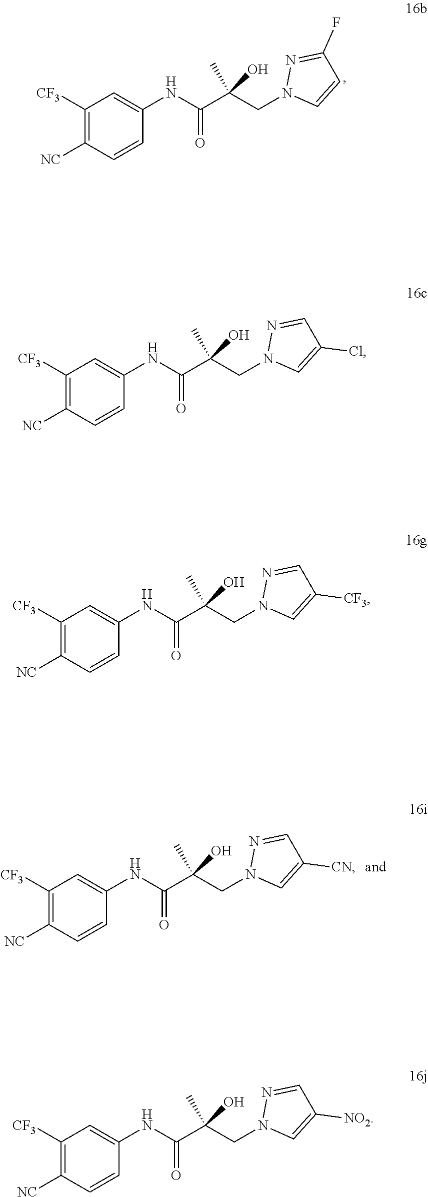 [SMILES: C[C@](O)(Cn1ccc(F)n1)C(=O)Nc1ccc(C#N)c(C(F)(F)F)c1 ; C[C@](O)(Cn1cc(Cl)cn1)C(=O)Nc1ccc(C#N)c(C(F)(F)F)c1 ; C[C@](O)(Cn1cc(C(F)(F)F)cn1)C(=O)Nc1ccc(C#N)c(C(F)(F)F)c1 ; C[C@](O)(Cn1cc(C#N)cn1)C(=O)Nc1ccc(C#N)c(C(F)(F)F)c1 ; C[C@](O)(Cn1cc([N+](=O)[O-])cn1)C(=O)Nc1ccc(C#N)c(C(F)(F)F)c1]

In some embodiments, the compound is represented by compound 21a or 21c.

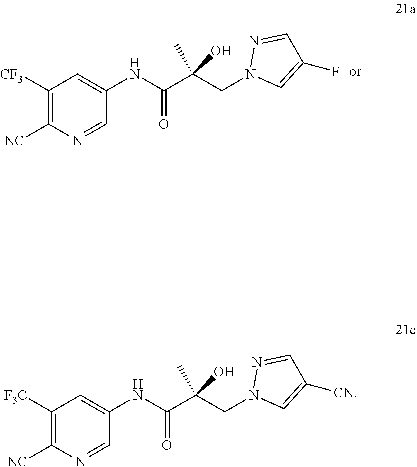 [SMILES: C[C@](O)(Cn1cc(F)cn1)C(=O)Nc1cnc(C#N)c(C(F)(F)F)c1 ; C[C@](O)(Cn1cc(C#N)cn1)C(=O)Nc1cnc(C#N)c(C(F)(F)F)c1]

In some embodiments, the compound is represented by any one of the following compounds

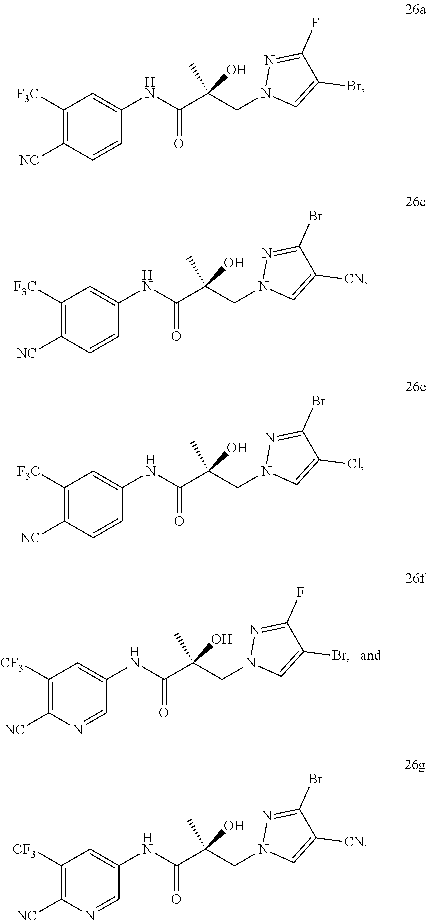 [SMILES: C[C@](O)(Cn1cc(Br)c(F)n1)C(=O)Nc1ccc(C#N)c(C(F)(F)F)c1 ; C[C@](O)(Cn1cc(C#N)c(Br)n1)C(=O)Nc1ccc(C#N)c(C(F)(F)F)c1 ; C[C@](O)(Cn1cc(Cl)c(Br)n1)C(=O)Nc1ccc(C#N)c(C(F)(F)F)c1 ; C[C@](O)(Cn1cc(Br)c(F)n1)C(=O)Nc1cnc(C#N)c(C(F)(F)F)c1 ; "and" ; C[C@](O)(Cn1cc(C#N)c(Br)n1)C(=O)Nc1cnc(C#N)c(C(F)(F)F)c1]

As used herein, the term “alkyl” refers to a saturated aliphatic hydrocarbon, straight-chained or branched-chained. The alkyl group may have 1-12 carbons, 1-7 carbons, 1-6 carbons, or 1-4 carbon atoms. In some embodiments, the alkyl group may be substituted with halogen, haloalkyl, hydroxyl, alkoxy carbonyl, amido, alkylamido, dialkylamido, nitro, CN, amino, alkylamino, dialkylamino, carboxyl, thio, or thioalkyl.

An “arylalkyl” group refers to an alkyl bound to an aryl, wherein alkyl and aryl are as defined herein. An example of an arylalkyl group is a benzyl group.

An “alkynyl” group refers to an unsaturated straight or branched hydrocarbon having one or more triple bonds. The alkynyl group may have 2-12 carbons. In some embodiments, the alkynyl group has 2-6 carbons or 2-4 carbons. Examples of alkynyl groups include, but are not limited to, ethynyl, propynyl, or butynyl, etc. The alkynyl group may be substituted with halogen, hydroxy, alkoxy carbonyl, amido, alkylamido, dialkylamido, nitro, amino, alkylamino, dialkylamino, carboxyl, thio, or thioalkyl.

As used herein, the term “aryl” group refers to an aromatic group having at least one carbocyclic aromatic group, which may be unsubstituted or substituted. The substituents include, but are not limited to, halogen, haloalkyl, hydroxy, alkoxy carbonyl, amido, alkylamido, dialkylamido, nitro, amino, alkylamino, dialkylamino, carboxy, thio, or thioalkyl. Nonlimiting examples of aryl rings are phenyl and naphthyl. The aryl group may be a 6-12 membered ring. In some embodiments, the aryl group may be a phenyl group.

The term “heteroaryl” refers to an aromatic group having at least one heterocyclic aromatic ring. In one embodiment, the heteroaryl comprises at least one heteroatom such as sulfur, oxygen, nitrogen, silicon, phosphorous or any combination thereof, as part of the ring. In another embodiment, the heteroaryl may be unsubstituted or substituted by one or more groups selected from halogen, aryl, heteroaryl, cyano, haloalkyl, hydroxy, alkoxy carbonyl, amido, alkylamido, dialkylamido, nitro, amino, alkylamino, dialkylamino, carboxy, thio, or thioalkyl. Nonlimiting examples of heteroaryl rings are pyranyl, pyrrolyl, pyrazinyl, pyrimidinyl, pyrazolyl, pyridinyl, furanyl, thiophenyl, thiazolyl, indolyl, imidazolyl, isoxazolyl, and the like. In one embodiment, the heteroaryl group is a 5-12 membered ring. In one embodiment, the heteroaryl group is a five membered ring. In one embodiment, the heteroaryl group is a six membered ring. In another embodiment, the heteroaryl group is a 5-8 membered ring. In another embodiment, the heteroaryl group comprises of 1-4 fused rings. In one embodiment, the heteroaryl group is 1,2,3-triazole. In one embodiment the heteroaryl is a pyridyl. In one embodiment the heteroaryl is a bipyridyl. In one embodiment the heteroaryl is a terpyridyl.

As used herein, the term “haloalkyl” group refers to an alkyl group that is substituted by one or more halogen atoms, e.g., by F, Cl, Br, or I.

A “hydroxyl” group refers to an OH group.

The term “halogen” or “halo” or “halide” refers to a halogen, e.g., F, Cl, Br, or I.

As used herein, in some embodiments, the term “pyrazole compound” may refer to “pyrazolylpropanamide compound.” In some embodiments, the terms “pyrazole propanamide” and “pyrazolylpropanamide” may be used interchangeably.

In one embodiment, this invention provides the use of pyrazolylpropanamide compounds as described herein, or its derivative, optical isomer, isomer, metabolite, pharmaceutically acceptable salt, pharmaceutical product, hydrate, N-oxide, prodrug, polymorph, crystal or combinations thereof.

In one embodiment, the methods of this invention make use of “pharmaceutically acceptable salts” of the pyrazolylpropanamide compounds, which may be produced, by reaction of the compounds with an acid or base.

The pyrazolylpropanamide compounds as described herein may be converted into pharmaceutically acceptable salts. A pharmaceutically acceptable salt may be produced by reaction of a compound with an acid or base.

Suitable pharmaceutically acceptable salts of amines may be prepared from an inorganic acid or from an organic acid. Examples of inorganic salts of amines include, but are not limited to, bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphates, sulfates, sulfamates, sulfanilates, sulfonic acids (alkylsulfonates, arylsulfonates, halogen substituted alkylsulfonates, halogen substituted arylsulfonates), sulfonates, or thiocyanates.

Examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, carboxylates, citrates, camphorates, camphorsulfonates, cyclohexyl sulfamates, cyclopentane propionates, calcium edetates, camsylates, carbonates, clavulanates, cinnamates, dicarboxylates, digluconates, dodecyl sulfonates, dihydrochlorides, decanoates, enanthuates, ethane sulfonates, edetates, edisylates, estolates, esylates, fumarates, formates, fluorides, galacturonates, gluconates, glutamates, glycolates, glucorates, glucoheptanoates, glycerophosphates, gluceptates, glycollylarsanilates, glutarates, glutamates, heptanoates, hexanoates, hydroxymaleates, hydroxycarboxylic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, nitrates, naphthalenesulfonates, 2-naphthalenesulfonates, nicotinates, napsylates, N-methylglucamines, oxalates, octanoates, oleates, pamoates, phenylacetates, picrates, phenylbenzoates, pivalates, propionates, phthalates, pectinates, phenylpropionates, palmitates, pantothenates, polygalacturates, pyruvates, quinates, salicylates, succinates, stearates, sulfanilates, subacetates, tartarates, theophylline acetates, p-toluene sulfonates (tosylates), trifluoroacetates, terephthalates, tannates, teoclates, trihaloacetates, triethiodide, tricarboxylates, undecanoates and valerates. Examples of inorganic salts of carboxylic acids or phenols may be selected from ammonium, alkali metals, and alkaline earth metals. Alkali metals include, but are not limited to, lithium, sodium, potassium, or cesium. Alkaline earth metals include, but are not limited to, calcium, magnesium, aluminium; zinc, barium, cholines, or quaternary ammoniums. Examples of organic salts of carboxylic acids or phenols may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglumines, N-methyl-D-glucamines, N,N′-dibenzylethylenediamines, nicotinamides, organic amines, omithines, pyridines, picolines, piperazines, procaine, tris(hydroxymethyl)methylamines, triethylamines, triethanolamines, trimethylamines, tromethamines and ureas.

In various embodiments, the pharmaceutically acceptable salts of the pyrazolylpropanamide compounds as described herein include, but are not limited to, HCl salt, oxalic acid salt, tartaric acid salt, HBr salt, and succinic acid salt. Each represents a separate embodiment of this invention.

Salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion exchange resin.

The methods of the invention may use an uncharged compound or a pharmaceutically acceptable salt of the compound. In particular, the methods use pharmaceutically acceptable salts of compounds of formulas I, II, IIA, IIB, III, IIIA, IIIB, IV, IVA, IVB, V, VA, VB, 10, 16a-16x, 21a-21j, 26a-26h, and 29a-29r. The pharmaceutically acceptable salt may be an amine salt or a salt of a phenol of the compounds of formulas I, II, IIA, IIB, III, IIIA, IIIB, IV, IVA, IVB, V, VA, VB, 10, 16a-16x, 21a-21j, 26a-26h, and 29a-29r.

In one embodiment, the methods of this invention make use of a free base, free acid, non charged or non-complexed compounds of formulas I, II, IIA, IIB, III, IIIA, IIIB, IV, IVA, IVB, V, VA, VB, 10, 16a-16x, 21a-21j, 26a-26h, and 29a-29r, and/or its isomer, optical isomer, or any mixture of optical isomers, pharmaceutical product, hydrate, polymorph, or combinations thereof.

In one embodiment, the methods of this invention make use of an optical isomer of a compound of formulas I, II, IIA, IIB, III, IIIA, IIIB, IV, IVA, IVB, V, VA, VB, 10, 16a-16x, 21a-21j, 26a-26h, and 29a-29r. In one embodiment, the methods of this invention make use of an isomer of a compound of formulas I, II, IIA, IIB, III, IIIA, IIIB, IV, IV A, IVB, V, VA, VB, 10, 16a-16x, 21a-21j, 26a-26h, and 29a-29r. In one embodiment, the methods of this invention make use of a pharmaceutical product of a compound of formulas I, II, IIA, IIB, III, IIIA, IIIB, IV, IVA, IVB, V, VA, VB, 10, 16a-16x, 21a-21j, 26a-26h, and 29a-29r. In one embodiment, the methods of this invention make use of a hydrate of a compound of formulas I, II, IIA, IIB, III, IIIA, IIIB, IV, IV A, IVB, V, VA, VB, 10, 16a-16x, 21a-21j, 26a-26h, and 29a-29r. In one embodiment, the methods of this invention make use of a polymorph of a compound of formulas I, II, IIA, IIB, III, IIIA, IIIB, IV, IVA, IVB, V, VA, VB, 10, 16a-16x, 21a-21j, 26a-26h, and 29a-29r. In one embodiment, the methods of this invention make use of a metabolite of a compound of formulas I, II, IIA, IIB, III, IIIA, IIIB, IV, IVA, IVB, V, VA, VB, 10, 16a-16x, 21a-21j, 26a-26h, and 29a-29r. In another embodiment, the methods of this invention make use of a composition comprising a compound of formulas I, II, IIA, IIB, III, IIIA, IIIB, IV, IV A, IVB, V, VA, VB, 10, 16a-16x, 21a-21j, 26a-26h, and 29a-29r, as described herein, or, in another embodiment, a combination of isomer, optical isomer, pharmaceutically acceptable salt, metabolite, pharmaceutical product, hydrate, polymorph of a compound of formulas I, II, IIA, IIB, III, IIIA, IIIB, IV, IV A, IVB, V, VA, VB, 10, 16a-16x, 21a-21j, 26a-26h, and 29a-29r.

As used herein, the term “isomer” includes, but is not limited to, optical isomers, structural isomers, or conformational isomers.

The term “isomer” is meant to encompass optical isomers of pyrazolylpropanamide compounds as described herein. It will be appreciated by those skilled in the art that the pyrazolylpropanamide compounds as described herein contain at least one chiral center. Accordingly, the compounds may exist as optically-active (such as an (R) isomer or (S) isomer) or racemic forms. Optically active compounds may exist as enantiomerically enriched mixtures. Some compounds may also exhibit polymorphism. It is to be understood that the present invention encompasses any racemic, optically active, polymorphic, or stereroisomeric form, or mixtures thereof. Thus, the invention may use pyrazolylpropanamide compounds as pure (R)isomers or as pure (S)-isomers. It is known in the art how to prepare optically active forms. For example, by resolution of the racemic form by recrystallization techniques, by synthesis from optically active starting materials, by chiral synthesis, or by chromatographic separation using a chiral stationary phase.

Pyrazolylpropanamide compounds as described herein may be hydrates of the compounds. As used herein, the term “hydrate” includes, but is not limited to, hemihydrate, monohydrate, dihydrate, or trihydrate. The invention also includes use of N-oxides of the amino substituents of the compounds described herein.

This invention may use of metabolites of the pyrazolylpropanamide compounds as described herein. In one embodiment, “metabolite” means any substance produced from another substance by metabolism or a metabolic process.

In some embodiments, pyrazolylpropanamide compounds as described herein can be prepared by any methods as known in the art. In other embodiments, the pyrazolylpropanamide compounds as described herein are prepared based on the synthetic methods in Example 1.

The pyrazolylpropanamide compounds described herein are found possessing favorable in vitro screening profiles, advantageous in vivo PK properties in rats, improved potency and efficacy of in vivo pharmacodynamics of secondary sex organs such as seminal vesicles (SV) and ventral prostate (VP), and improved potency in in vivo models of antiandrogen resistant CRPC such as enzalutamide-resistant (termed as MDVR) VCaP xenografts. The in vivo antiandrogenicity of the pyrazolylpropanamide compounds as described herein has a potency and efficacy that far has exceeded previous generations of SARDs in a broad scope of preclinical models and infra. These data suggest that these pyrazolylpropanamide compounds have panantagonism properties and are highly potent and efficacious in vivo activity.

The pyrazolylpropanamide compounds as described herein possess advantages over direct (flutamide (1) bicalutamide (2), nilutamide (3), enzalutamide (4), apalutamide (5), or darolutamide (6)) or indirect (abiraterone acetate (7)) LBD targeted AR antagonists in that they inhibit and degrade all forms of AR protein tested thus far, thereby expanding the scope of CRPC models susceptible to inhibition compared to approved agents. Further, the pyrazolylpropanamide compounds as described herein exhibit excellent ADME and PK properties in vivo, allowing unprecedented xenograft efficacy in intact animals in models of antiandrogen resistance, and reported herein for similar molecules. The preclinical profile includes the ability to degrade (in most cases) and inhibit wtAR, AR point mutations, truncation mutants, AR overexpression (e.g., AR gene amplification), and combinations thereof, and improved in vivo PK and PD properties.

In one aspect, this invention provides a method of treating prostate cancer (PCa) in a subject in need thereof, comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating or inhibiting the progression of prostate cancer (PCa) or increasing the survival of a subject suffering from prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In some embodiments, the prostate cancer may depend on AR-FL and/or AR-SV for proliferation. The prostate cancer may be resistant to treatment with darolutamide, enzalutamide, apalutamide, bicalutamide, abiraterone, EPI-001, EPI-506, AZD-3514, galeterone, ASC-J9, flutamide, hydroxyflutamide, nilutamide, cyproterone acetate, ketoconazole, spironolactone, or any combination thereof.

The method of the invention may also reduce the levels of AR, AR-FL, AR-FL with antiandrogen resistance-conferring AR-LBD mutations, AR-SV, gene-amplified AR, or any combination thereof.

In some embodiments of the method of the invention, the prostate cancer is darolutamide resistant prostate cancer, enzalutamide resistant prostate cancer, apalutamide resistant prostate cancer, or abiraterone resistant prostate cancer.

In one embodiment, this invention provides a method of treating darolutamide resistant prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating or inhibiting the progression of darolutamide resistant prostate cancer (PCa) or increasing the survival of a subject suffering from apalutamide resistant prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In one embodiment, this invention provides a method of treating enzalutamide resistant prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating or inhibiting the progression of enzalutamide resistant prostate cancer or increasing the survival of a subject suffering from enzalutamide resistant prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In one embodiment, this invention provides a method of treating apalutamide resistant prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating or inhibiting the progression of apalutamide resistant prostate cancer (PCa) or increasing the survival of a subject suffering from apalutamide resistant prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In one embodiment, this invention provides a method of treating abiraterone resistant prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating or inhibiting the progression of abiraterone resistant prostate cancer or increasing the survival of a subject suffering from abiraterone resistant prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In some embodiments of the method of the invention, the prostate cancer is advanced prostate cancer, refractory prostate cancer, or castration-resistant prostate cancer, or castration-sensitive prostate cancer. In some embodiments, the castration resistant prostate cancer (CRPC) is metastatic CRPC (mCRPC), non-metastatic CRPC (nmCRPC), or high-risk nmCRPC.

The invention provides a method of treating advanced prostate cancer in a subject in need thereof comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating or inhibiting the progression of advanced prostate cancer (PCa) or increasing the survival of a subject suffering from advanced prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention provides a method of treating refractory prostate cancer in a subject in need thereof comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating or inhibiting the progression of refractory prostate cancer (PCa) or increasing the survival of a subject suffering from refractory prostate cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention provides a method of treating castration resistant prostate cancer in a subject in need thereof comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In some embodiments of the method of the invention, the method further comprises administering androgen deprivation therapy to the subject.

The invention encompasses a method of treating or inhibiting the progression of castration resistant prostate cancer or increasing the survival of a subject suffering from castration resistant prostate cancer (CRPC) comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In some embodiments of the method of the invention, the method further comprises administering androgen deprivation therapy to the subject.

In some embodiments, the method further comprises a second therapy such as androgen deprivation therapy (ADT) or 1HRH agonist or antagonist. 1HRH agonists include, but are not limited to, leuprolide acetate.

As used herein, the term “increase the survival” refers to a lengthening of time when describing the survival of a subject. Thus, in this context, the pyrazolylpropanamide compounds as described herein may be used to increase the survival of men with advanced prostate cancer, refractory prostate cancer, castration resistant prostate cancer (CRPC), metastatic CRPC (mCRPC), non-metastatic CRPC (nmCRPC), high-risk nmCRPC, or darolutamide resistant prostate cancer, enzalutamide resistant prostate cancer, apalutamide resistant prostate cancer, or abiraterone resistant prostate cancer.

Alternatively, as used herein, the terms “increase”, increasing”, or “increased” may be used interchangeably and refer to an entity becoming progressively greater (as in size, amount, number, or intensity), wherein for example the entity is sex hormone-binding globulin (SHBG) or prostate-specific antigen (PSA).

The compounds as described herein may be used for increasing metastasis-free survival (MFS) in a subject suffering from non-metastatic prostate cancer. The non-metastatic prostate cancer may be non-metastatic advanced prostate cancer, non-metastatic CRPC (nmCRPC), or high-risk nmCRPC.

The pyrazolylpropanamide compounds described herein may be used to provide a dual action. For example, the pyrazolylpropanamide compounds may treat prostate cancer and prevent metastasis. The prostate cancer may be refractory prostate cancer, advanced prostate cancer, castration resistant prostate cancer (CRPC), metastatic CRPC (mCRPC), non-metastatic CRPC (nmCRPC), or high-risk nmCRPC.

Men with advanced prostate cancer who are at high risk for progression to castration resistant prostate cancer (CRPC) are men on ADT with serum total testosterone concentrations greater than 20 ng/dL or men with advanced prostate cancer who at the time of starting ADT had either (1) confirmed Gleason pattern 4 or 5 prostate cancer, (2) metastatic prostate cancer, (3) a PSA doubling time<3 months, (4) a PSA 2:20 ng/mL, or (5) a PSA relapse in <3 years after definitive local therapy (radical prostatectomy or radiation therapy).

Normal levels of prostate specific antigen (PSA) are dependent on several factors, such as age and the size of a male subject's prostate, among others. PSA levels in the range between 2.5-10 ng/mL are considered “borderline high” while levels above 10 ng/mL are considered “high.” A rate change or “PSA velocity” greater than 0.75/year is considered high. PSA levels may increase despite ongoing ADT or a history of ADT, surgical castration or despite treatment with antiandrogens and/or 1HRH agonist.

Men with high-risk non-metastatic castration resistant prostate cancer (high-risk nmCRPC) may include those with rapid PSA doubling times, having an expected progression-free survival of approximately 18 months or less (Miller K, Moul J W, Gleave M, et al. 2013. “Phase III, randomized, placebo-controlled study of once-daily oral zibotentan (ZD4054) in patients with non-metastatic castration-resistant prostate cancer,” Prostate Cane Prost Dis. February; 16:187-192). This relatively rapid progression of their disease underscores the importance of novel therapies for these individuals.

The methods of the invention may treat subjects with PSA levels greater than 8 ng/mL where the subject suffers from high-risk nmCRPC. The patient population includes subjects suffering from nmCRPC where PSA doubles in less than 8 months or less than 10 months. The method may also treat patient populations where the total serum testosterone levels are greater than 20 ng/mL in a subject suffering from high-risk nmCRPC. In one case, the serum free testosterone levels are greater than those observed in an orchiectomized male in a subject suffering from high-risk nmCRPC.

Treatment of prostate cancer, advanced prostate cancer, CRPC, mCRPC, nmCRPC darolutamide, resistant prostate cancer, enzalutamide resistant prostate cancer, apalutamide resistant prostate cancer, and/or abiraterone resistant prostate cancer may result in clinically meaningful improvement in prostate cancer related symptoms, function and/or survival. Clinically meaningful improvement can be determined by an increase in radiographic progression free survival (rPFS) if cancer is metastatic, or an increase metastasis-free survival (MFS) if cancer is non-metastatic, among others.

The invention encompasses methods of lowering serum prostate specific antigen (PSA) levels in a male subject suffering from prostate cancer, advanced prostate cancer, metastatic prostate cancer, castration resistant prostate cancer (CRPC), darolutamide resistant prostate cancer, enzalutamide resistant prostate cancer, apalutamide resistant prostate cancer, or abiraterone resistant prostate cancer, comprising administering a therapeutically effective amount of a compound, wherein the compound is represented by the structure of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of secondary hormonal therapy that reduces serum PSA in a male subject suffering from castration resistant prostate cancer (CRPC) comprising administering a therapeutically effective amount of a compound of formulas 10, 16a-16x, 21a-21j, 26a-26h, and 29a-29r that reduces serum PSA in a male subject suffering from castration resistant prostate cancer.

The invention encompasses a method of reducing levels of AR, AR-full length (ARFL), AR-FL with antiandrogen resistance-conferring AR-LBD mutations, AR-splice variant (AR-SV), and/or amplifications of the AR gene within the tumor in the subject in need thereof comprising administering a therapeutically effective amount of a compound of formulas 10, 16a-16x, 21a-21j, 26a-26h, and 29a-29r to reduce the level of AR, AR-full length (AR-FL), AR-FL with antiandrogen resistance-conferring AR-LBD or other AR mutations, AR-splice variant (ARSV), and/or amplifications of the AR gene within the tumor.

The method may increase radiographic progression free survival (rPFS) or metastasis-free survival (MFS).

Subjects may have non-metastatic cancer; failed androgen deprivation therapy (ADT), undergone orchidectomy, or have high or increasing prostate specific antigen (PSA) levels; subjects may be a patient with prostate cancer, advanced prostate cancer, refractory prostate cancer, CRPC patient, metastatic castration resistant prostate cancer (mCRPC) patient, nonmetastatic castration resistant prostate cancer (nmCRPC) patient, darolutamide resistant prostate cancer, or enzalutamide resistant prostate cancer, apalutamide resistant prostate cancer, or abiraterone resistant prostate cancer. In these subjects, the nmCRPC may be high-risk nmCRPC. Further the subject may be on androgen deprivation therapy (ADT) with or without castrate levels of total T.

As used herein, the phrase “a subject suffering from castration resistant prostate cancer” refers to a subject with at least one of the following characteristics: has been previously treated with androgen deprivation therapy (ADT); has responded to the ADT and currently has a serum PSA>2 ng/mL or >2 ng/mL and representing a 25% increase above the nadir achieved on the ADT; a subject which despite being maintained on androgen deprivation therapy is diagnosed to have serum PSA progression; a castrate level of serum total testosterone (<50 ng/dL) or a castrate level of serum total testosterone (<20 ng/dL). The subject may have rising serum PSA on two successive assessments at least 2 weeks apart; been effectively treated with ADT; or has a history of serum PSA response after initiation of ADT.

As used herein, the term “serum PSA progression” refers to a 25% or greater increase in serum PSA and an absolute increase of 2 ng/ml or more from the nadir; or to serum PSA>2 ng/mL, or >2 ng/mL and a 25% increase above the nadir after the initiation of androgen deprivation therapy (ADT). The term “nadir” refers to the lowest PSA level while a patient is undergoing ADT.

The term “serum PSA response” refers to at least one of the following: at least 90% reduction in serum PSA value prior to the initiation of ADT; to <10 ng/mL undetectable level of serum PSA (<0.2 ng/mL) at any time; at least 50% decline from baseline in serum PSA; at least 90% decline from baseline in serum PSA; at least 30% decline from baseline in serum PSA; or at least 10% decline from baseline in serum PSA.

The methods of this invention comprise administering a combination of forms of ADT and a pyrazolylpropanamide compound as described herein, e.g., a compound of formulas 10, 16a-16x, 21a-21j, 26a-26h, and 29a-29r. Forms of ADT include a 1HRH agonist. 1HRH agonist includes, but is not limited to, leuprolide acetate (Lupron®) (U.S. Pat. Nos. 5,480,656; 5,575,987; 5,631,020; 5,643,607; 5,716,640; 5,814,342; and 6,036,976 hereby incorporated by reference) or goserelin acetate (Zoladex®) (U.S. Pat. Nos. 7,118,552; 7,220,247; and 7,500,964 hereby incorporated by reference). Forms of ADT include, but are not limited to 1HRH antagonists, reversible antiandrogens, or bilateral orchidectomy. 1HRH antagonists include, but are not limited to, degarelix and abarelix. Antiandrogens include, but are not limited to, bicalutamide, flutamide, hydroxyflutamide, finasteride, dutasteride, enzalutamide, apalutamide, EPI-001, EPI-506, darolutamide, nilutamide, chlormadinone, abiraterone, or any combination thereof. In some embodiments, the methods of the invention encompass administering at least a pyrazolylpropanamide compound as described herein, e.g., a compound of formulas 10, 16a-16x, 21a-21j, 26a-26h, and 29a-29r, and a lyase inhibitor (e.g., abiraterone).

The term “advanced prostate cancer” refers to metastatic cancer having originated in the prostate and having widely metastasized to beyond the prostate such as the surrounding tissues to include the seminal vesicles the pelvic lymph nodes or bone, or to other parts of the body. Prostate cancer pathologies are graded with a Gleason grading from 1 to 5 in order of increasing malignancy. Patients with significant risk of progressive disease and/or death from prostate cancer should be included in the definition and any patient with cancer outside the prostate capsule with disease stages as low as IIB clearly has “advanced” disease. “Advanced prostate cancer” can refer to locally advanced prostate cancer.

The term “refractory” may refer to cancers that do not respond to treatment. E.g., prostate or breast cancer may be resistant at the beginning of treatment or it may become resistant during treatment. “Refractory cancer” may also be referred to herein as “resistant cancer”.

The term “castration resistant prostate cancer” (CRPC) refers to advanced prostate cancer that is worsening or progressing while the patient remains on ADT or other therapies to reduce testosterone, or prostate cancer which is considered hormone refractory, hormone naive, androgen independent or chemical or surgical castration resistant. CRPC may be the result of AR activation by intracrine androgen synthesis; expression of AR splice variants (AR-SV) that lack ligand binding domain (LBD); or expression of AR-LBD or other AR mutations with potential to resist antagonists. Castration resistant prostate cancer (CRPC) is an advanced prostate cancer which developed despite ongoing ADT and/or surgical castration. Castration resistant prostate cancer is defined as prostate cancer that continues to progress or worsen or adversely affect the health of the patient despite prior surgical castration, continued treatment with gonadotropin releasing hormone agonists (e.g., leuprolide) or antagonists (e.g., degarelix or abarelix), antiandrogens (e.g., bicalutamide, flutamide, enzalutamide, apalutamide, darolutamide, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®), cabozantinib (Cometriq™, also known as XL184)) or other prostate cancer therapies (e.g., vaccines (sipuleucel-T (Provenge®), GV AX, etc.), herbal (PC-SPES) and lyase inhibitor (abiraterone)) as evidenced by increasing or higher serum levels of prostate specific antigen (PSA), metastasis, bone metastasis, pain, lymph node involvement, increasing size or serum markers for tumor growth, worsening diagnostic markers of prognosis, or patient condition.

Castration resistant prostate cancer may be defined as hormone naïve prostate cancer. In men with castration resistant prostate cancer, the tumor cells may have the ability to grow in the absence of androgens (hormones that promote the development and maintenance of male sex characteristics).

Many early prostate cancers require androgens for growth, but advanced prostate cancers are androgen-independent or hormone naïve.

The term “androgen deprivation therapy” (ADT) may include orchiectomy; administering luteinizing hormone-releasing hormone (LHRH) analogs; administering luteinizing hormone-releasing hormone (LHRH) antagonists; administering 5a-reductase inhibitors; administering antiandrogens; administering inhibitors of testosterone biosynthesis; administering estrogens; or administering 17a-hydroxy lase/C 17,20 lyase (CYP17A1) inhibitors. 1HRH drugs lower the amount of testosterone made by the testicles. Examples of 1HRH analogs available in the United States include leuprolide (Lupron®, Viadur®, Eligard®), goserelin (Zoladex®), triptorelin (Trelstar®), and histrelin (Vantas®). Antiandrogens block the body's ability to use any androgens. Examples of antiandrogens drugs include darolutamide (Nubega®), enzalutamide (Xtandi®), apalutamide (Erleada®), flutamide (Eulexin®), bicalutamide (Casodex®), and nilutamide (Nilandron®). Luteinizing hormone-releasing hormone (LHRH) antagonists include abarelix (Plenaxis®) or degarelix (Firmagon®) (approved for use by the FDA in 2008 to treat advanced prostate cancer). 5a-Reductase inhibitors block the body's ability to convert testosterone to the more active androgen, 5a-dihydrotestosterone (DHT) and include drugs such as finasteride (Proscar®) and dutasteride (A Avodart®). Inhibitors of testosterone biosynthesis include drugs such as ketoconazole (Nizoral®). Estrogens include diethylstilbestrol or 17β-estradiol. 17α-Hydroxylase/C17, 20 lyase (CYP17A1) inhibitors include abiraterone (Zytiga®).

The invention encompasses a method of treating antiandrogen-resistant prostate cancer, comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a. In some embodiments, the antiandrogen may include, but is not limited to, bicalutamide, hydroxyflutamide, flutamide, darolutamide, enzalutamide, apalutamide, and/or abiraterone.

The invention encompasses a method of treating prostate cancer in a subject in need thereof, wherein said subject has a rearranged AR, AR overexpressing prostate cancer, castration-resistant prostate cancer, castration-sensitive prostate cancer, AR-V7 expressing prostate cancer, or d567ES expressing prostate cancer, comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In one embodiment, the castration-resistant prostate cancer is a rearranged AR, AR overexpressing castration-resistant prostate cancer, F876L mutation expressing castration-resistant prostate cancer, F876L_T877 A double mutation expressing castration-resistant prostate cancer, AR-V7 expressing castration-resistant prostate cancer, d567ES expressing castration-resistant prostate cancer, and/or castration-resistant prostate cancer characterized by intratumoral androgen synthesis.

In one embodiment, the castration-sensitive prostate cancer is F876L mutation expressing castration-sensitive prostate cancer, F876L_T877 A double mutation castration-sensitive prostate cancer, and/or castration-sensitive prostate cancer characterized by intratumoral androgen synthesis.

In one embodiment, the treating of castration-sensitive prostate cancer is conducted in a non-castrate setting, or as monotherapy, or when castration-sensitive prostate cancer tumor is resistant to darolutamide, enzalutamide, apalutamide, and/or abiraterone.

The invention encompasses a method of treating AR overexpressing prostate cancer in a subject in need thereof, comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating castration-resistant prostate cancer in a subject in need thereof, comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a. In one embodiment, the castration-resistant prostate cancer is a rearranged AR, AR overexpressing castration-resistant prostate cancer, F876L mutation expressing castration-resistant prostate cancer, F876L_T877 A double mutation expressing castration-resistant prostate cancer, AR-V7 expressing castration-resistant prostate cancer, d567ES expressing castration-resistant prostate cancer, and/or castration-resistant prostate cancer characterized by intratumoral androgen synthesis.

The invention encompasses a method of treating castration-sensitive prostate cancer in a subject in need thereof, comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a. In one embodiment, the castration-sensitive prostate cancer is F876L mutation expressing castration-sensitive prostate cancer, F876L_T877 A double mutation castration-sensitive prostate cancer, and/or castration-sensitive prostate cancer characterized by intratumoral androgen synthesis. In one embodiment, the treating of castration-sensitive prostate cancer is conducted in a non-castrate setting, or as monotherapy, or when castration-sensitive prostate cancer tumor is resistant to darolutamide, enzalutamide, apalutamide, and/or abiraterone.

The invention encompasses a method of treating AR-V7 expressing prostate cancer in a subject in need thereof, comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating d567ES expressing prostate cancer in a subject in need thereof, comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

In another aspect, the invention provides a method of treating an androgen receptor dependent disease or condition or an androgen dependent disease or condition in a subject in need thereof, comprising administering to the subject a therapeutically effective amount of a pyrazolylpropanamide compound as described herein, e.g., a compound of formulas I, II, IIA, IIB, III, IIIA, IIIB, IV, IV A, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

As used herein, the term “androgen receptor associated conditions” or “androgen sensitive diseases or disorders” or “androgen-dependent diseases or disorders” are conditions, diseases, or disorders that are modulated by or whose pathogenesis is dependent upon the activity of the androgen receptor. The androgen receptor is expressed in most tissues of the body however it is overexpressed in, inter alia, the prostate and skin. ADT has been the mainstay of prostate cancer treatment for many years, and pyrazolylpropanamide compound as described herein may also be useful in treating various prostate cancers, benign prostatic hypertrophy, prostamegaly, and other maladies of the prostate. In one embodiment, an “androgen receptor dependent disease or condition” is a medical condition that is, in part or in full, dependent on, or is sensitive to, the presence of androgenic activity or activation of the AR-axis in the body. In one embodiment, an androgen dependent disease or condition is used interchangeably with and androgen receptor dependent disease or condition.

The invention encompasses methods of treating benign prostatic hypertrophy comprising administering to the subject a therapeutically effective amount of a pyrazolylpropanamide compound as described herein, e.g., a compound of formulas I, II, IIA, IIB, III IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses methods of treating prostamegaly comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses methods of treating hyperproliferative prostatic disorders and diseases comprising administering to the subject a therapeutically effective amount of a pyrazolylpropanamide compound as described herein, e.g., a compound of formulas I, II, IIA, IIB, III, IIIA, IIIB, IV, IV A, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The term “reducing the pathogenesis” is to be understood to encompass reducing tissue damage, or organ damage associated with a particular disease, disorder or condition. The term may include reducing the incidence or severity of an associated disease, disorder or condition, with that in question or reducing the number of associated diseases, disorders or conditions with the indicated, or symptoms associated thereto.

Triple negative breast cancer (TNBC) is a type of breast cancer lacking the expression of the estrogen receptor (ER), progesterone receptor (PR), and HER2 receptor kinase. TNBC lacks the hormone and kinase therapeutic targets used to treat other types of primary breast cancers. Chemotherapy is often the initial pharmacotherapy for TNBC. AR is often still expressed in TNBC and may offer a hormone targeted therapeutic alternative to chemotherapy. In ER-positive breast cancer, AR is a positive prognostic indicator as it is believed that activation of AR limits and/or opposes the effects of the ER in breast tissue and tumors. In the absence of ER, it is possible that AR actually supports the growth of breast cancer tumors. Though the role of AR is not fully understood in TNBC, certain TNBC's may be supported by androgen independent activation of AR-SVs lacking the LBD or androgen-dependent activation of AR full length. Darolutamide, enzalutamide, apalutamide, and other LED-directed traditional AR antagonists would not be able to antagonize AR-SVs in these TNBC's. Pyrazolylpropanamide compounds as described herein, which are capable of destroying AR-SVs through a binding site in the NTD of AR, would be able to antagonize AR including AR-SV observed in TNBC patient derived xenografts and provide an anti-tumor effect.

In one embodiment, the invention provides a method of treating triple negative breast cancer (TNBC) comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The invention encompasses a method of treating or inhibiting the progression of triple negative breast cancer (TNBC) or increasing the survival of a subject suffering from triple negative breast cancer comprising administering to the subject a therapeutically effective amount of a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r, or its optical isomer, pharmaceutically acceptable salt, hydrate or any combination thereof. In some embodiments, the pyrazolylpropanamide compound is represented by a compound of formulas 10, 16a-16x, 21a-21j, 26a-26h, and 29a-29r. In some embodiments, the pyrazolylpropanamide compound is compound 16b, 16c, 16g, 16i, 16j, 21a, 21c. 26a, 26c, 26e, 26f, or 26g. In other embodiments, the pyrazolylpropanamide compound is compound 26a.

The pyrazolylpropanamide compounds as described herein may be used m pharmaceutical compositions. As used herein, “pharmaceutical composition” means either the compound or pharmaceutically acceptable salt of the active ingredient with a pharmaceutically acceptable carrier or diluent. A “therapeutically effective amount” as used herein refers to that amount which provides a therapeutic effect for a given indication and administration regimen.

The pharmaceutical compositions containing pyrazolylpropanamide compound as described herein may further comprise at least one 1HRH agonist or antagonist, antiandrogen, anti-programmed death receptor 1 (anti-PD-1) drug or anti-PD-L1 drug. 1HRH agonists include, but are not limited to, leuprolide acetate (Lupron®) (U.S. Pat. Nos. 5,480,656; 5,575,987; 5,631,020; 5,643,607; 5,716,640; 5,814,342; and 6,036,976 hereby incorporated by reference) or goserelin acetate (Zoladex®) (U.S. Pat. Nos. 7,118,552; 7,220,247; and 7,500,964 hereby incorporated by reference). 1HRH antagonists include, but are not limited to, degarelix or abarelix. Antiandrogens include, but are not limited to, bicalutamide, flutamide, finasteride, dutasteride, darolutamide, enzalutamide, apalutamide, nilutamide, chlormadinone, abiraterone, or any combination thereof. Anti-PD-1 drugs include, but are not limited to, AMP-224, nivolumab, pembrolizumab, pidilizumab, and AMP-554. Anti-PD-L1 drugs include, but are not limited to, BMS-936559, atezolizumab, durvalumab, avelumab, and MPDL3280A. Anti-CTLA-4 drugs include, but are not limited to, ipilimumab and tremelimumab.

As used herein, the term “administering” refers to bringing a subject in contact with a compound of the present invention. As used herein, administration can be accomplished in vitro, i.e., in a test tube, or in vivo, i.e., in cells or tissues of living organisms, for example humans. The subjects may be a male or female subject or both.

Numerous standard references are available that describe procedures for preparing various compositions or formulations suitable for administration of the compounds of the invention. Examples of methods of making formulations and preparations can be found in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage form are closely related to the therapeutic amounts of the compounds or compositions which are desirable and efficacious for the given treatment application.

The pharmaceutical compositions of pyrazolylpropanamide compounds as described herein can be administered to a subject by any method known to a person skilled in the art. These methods include, but are not limited to, orally, parenterally, intravascularly, paracancerally, transmucosally, transdermally, intramuscularly, intranasally, intravenously, intradermally, subcutaneously, sub lingually, intraperitoneally, intraventricularly, intracranially, intra vaginally, by inhalation, rectally, or intratumorally. These methods include any means in which the composition can be delivered to tissue (e.g., needle or catheter). Alternatively, a topical administration may be desired for application to dermal, ocular, or mucosa! surfaces. Another method of administration is via aspiration or aerosol formulation. The pharmaceutical compositions may be administered topically to body surfaces and are thus formulated in a form suitable for topical administration. Suitable topical formulations include gels, ointments, creams, lotions, drops and the like. For topical administrations, the compositions are prepared and applied as solutions, suspensions, or emulsions in a physiologically acceptable diluent with or without a pharmaceutical carrier.

Suitable dosage forms include, but are not limited to, oral, rectal, sub-lingual, mucosa!, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. Depending on the indication, formulations suitable for oral or topical administration are preferred.

Topical Administration: The pyrazolylpropanamide compounds as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r may be administered topically. As used herein, “topical administration” refers to application of the compounds of formulas I, II, IIA, IIB, III, IIIA, IIIB, IV, IVA, IVB, V, VA, VB, 10, 16a-16x, 21a-21j, 26a-26h, and 29a-29r (and optional carrier) directly to the skin and/or hair. The topical composition can be in the form of solutions, lotions, salves, creams, ointments, liposomes, sprays, gels, foams, roller sticks, and any other formulation routinely used in dermatology.

Topical administration is used for indications found on the skin, such as hirsutism, alopecia, acne, and excess sebum. The dose will vary, but as a general guideline, the compound will be present in a dermatologically acceptable carrier in an amount of from about 0.01 to 50 w/w %, and more typically from about 0.1 to 10 w/w %. Typically, the dermatological preparation will be applied to the affected area from 1 to 4 times daily. “Dermatologically acceptable” refers to a carrier which may be applied to the skin or hair, and which will allow the drug to diffuse to the site of action. More specifically “site of action”, it refers to a site where inhibition of androgen receptor or degradation of the androgen receptor is desired.

The compositions of the invention may also include solid preparations such as cleansing soaps or bars. These compositions are prepared according to methods known in the art.

Formulations such as aqueous, alcoholic, or aqueous-alcoholic solutions, or creams, gels, emulsions or mousses, or aerosol compositions with a propellant may be used to treat indications that arise where hair is present. Thus, the composition can also be a hair care composition. Such hair care compositions include, but are not limited to, shampoo, a hair-setting lotion, a treating lotion, a styling cream or gel, a dye composition, or a lotion or gel for preventing hair loss. The amounts of the various constituents in the dermatological compositions are those conventionally used in the fields considered.

Medicinal and cosmetic agents containing the pyrazolylpropanamide compounds as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r will typically be packaged for retail distribution (i.e., an article of manufacture). Such articles will be labeled and packaged in a manner to instruct the patient how to use the product. Such instructions will include the condition to be treated, duration of treatment, dosing schedule, etc.

To prepare such pharmaceutical dosage forms, the active ingredient may be mixed with a pharmaceutical carrier according to conventional pharmaceutical compounding techniques. The carrier may take a wide variety of forms depending on the form of preparation desired for administration.

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethyl acrylate), calcium carbonate, magnesium oxide, talc, or mixtures thereof.

Oral and Parenteral Administration: In preparing the compositions in oral dosage form, any of the usual pharmaceutical media may be employed. Thus, for liquid oral preparations, such as, suspensions, elixirs, and solutions, suitable carriers and additives include water, glycols, oils, alcohols, flavoring agents, preservatives, coloring agents, and the like. For solid oral preparations such as, powders, capsules, and tablets, suitable carriers and additives include starches, sugars, diluents, granulating agents, lubricants, binders, disintegrating agents, and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients may be included, such as ingredients that aid solubility or for preservation. Injectable solutions may also be prepared in which case appropriate stabilizing agents may be employed.

In some applications, it may be advantageous to utilize the active agent m a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Methods of treatment using formulations suitable for oral administration may be presented as discrete units such as capsules, cachets, tablets, or lozenges, each containing a predetermined amount of the active ingredient. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration may comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosa! spray formulations (e.g., inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more ingredient selected from diluents, buffers, flavoring agents, binders, disintegrants, surface active agents, thickeners, lubricants, preservatives (including antioxidants), and the like.

The formulations may be of immediate release, sustained release, delayed-onset release or any other release profile known to one skilled in the art.

For administration to mammals, and particularly humans, it is expected that the physician will determine the actual dosage and duration of treatment, which will be most suitable for an individual and can vary with the age, weight, genetics and/or response of the particular individual.

The methods of the invention comprise administration of a compound at a therapeutically effective amount. The therapeutically effective amount may include various dosages.

In one embodiment, a pyrazolylpropanamide compound as described herein, e.g., a compound of formulas I, II, IIA, IIB, III, IIIA, IIIB, IV, IVA, IVB, V, VA, VB, 10, 16a-16x, 21a-21j, 26a-26h, and 29a-29r is administered at a dosage of 1-3000 mg per day. In additional embodiments, the pyrazoly propenamide compound is administered at a dose of 1-10 mg per day, 3-26 mg per day, 3-60 mg per day, 3-16 mg per day, 3-30 mg per day, 10-26 mg per day, 15-60 mg, 50-100 mg per day, 50-200 mg per day, 100-250 mg per day, 125-300 mg per day, 20-50 mg per day, 5-50 mg per day, 200-500 mg per day, 125-500 mg per day, 500-1000 mg per day, 200-1000 mg per day, 1000-2000 mg per day, 1000-3000 mg per day, 125-3000 mg per day, 2000-3000 mg per day, 300-1500 mg per day or 100-1000 mg per day. In one embodiment, the pyrazolylpropanamide compound is administered at a dosage of 25 mg per day. In one embodiment, the pyrazolylpropanamide compound is administered at a dosage of 40 mg per day. In one embodiment, the pyrazolylpropanamide compound is administered at a dosage of 50 mg per day. In one embodiment, the pyrazolylpropanamide compound is administered at a dosage of 67.5 mg per day. In one embodiment, the pyrazolylpropanamide compound is administered at a dosage of 75 mg per day. In one embodiment, the pyrazolylpropanamide compound is administered at a dosage of 80 mg per day. In one embodiment, the pyrazolylpropanamide compound is administered at a dosage of 100 mg per day. In one embodiment, the pyrazolylpropanamide compound is administered at a dosage of 125 mg per day. In one embodiment, the pyrazolylpropanamide compound is administered at a dosage of 250 mg per day. In one embodiment, the pyrazolylpropanamide compound is administered at a dosage of 300 mg per day. In one embodiment, the pyrazolylpropanamide compound is administered at a dosage of 500 mg per day. In one embodiment, the pyrazolylpropanamide compound is administered at a dosage of 600 mg per day. In one embodiment, the pyrazolylpropanamide compound is administered at a dosage of 1000 mg per day. In one embodiment, the pyrazolylpropanamide compound is administered at a dosage of 1500 mg per day. In one embodiment, the pyrazolylpropanamide compound is administered at a dosage of 2000 mg per day. In one embodiment, the pyrazolylpropanamide compound is administered at a dosage of 2500 mg per day. In one embodiment, a the pyrazolylpropanamide compound is administered at a dosage of 3000 mg per day.

The methods may comprise administering a compound at various dosages. For example, the compound may be administered at a dosage of 3 mg, 10 mg, 30 mg, 40 mg, 50 mg, 80 mg, 100 mg, 120 mg, 125 mg, 200 mg, 250 mg, 300 mg, 450 mg, 500 mg, 600 mg, 900 mg, 1000 mg, 1500 mg, 2000 mg, 2500 mg or 3000 mg.

Alternatively, the compound may be administered at a dosage of 0.1 mg/kg/day. The compound may be administered at a dosage between 0.2 to 30 mg/kg/day, or 0.2 mg/kg/day, 0.3 mg/kg/day, 1 mg/kg/day, 3 mg/kg/day, 5 mg/kg/day, 10 mg/kg/day, 20 mg/kg/day, 30 mg/kg/day, 50 mg/kg/day or 100 mg/kg/day.

The pharmaceutical composition may be a solid dosage form, a solution, or a transdermal patch. Solid dosage forms include, but are not limited to, tablets and capsules.

The following examples are presented in order to more fully illustrate the preferred embodiments of the invention. They should in no way, however, be construed as limiting the broad scope of the invention.

### EXAMPLES

### General Procedures, Materials, and Information.

All solvents and chemicals were used as purchased without further purification. The progress of all reactions was monitored by thin-layer chromatography (TLC) analysis on silica gel 60 F254 plates (Merck). Column chromatography was performed with a silica gel column [(Merck Kieselgel 60, 70-230 mesh, Merck).

General methods: All non-aqueous reactions were performed in oven-dried glassware under an inert atmosphere of dry nitrogen. All the reagents and solvents were purchased from Aldrich (St. Louis, Mo.), Alfa-Aesar (Ward Hill, Mass.), Cambi-Blocks (San Diego, Calif.), Ark Pharm (Libertyville, IL) and used without further purification. Analytical thin-layer chromatography was performed on Silica Gel GHLF 10×20 cm Analtech TLC Uniplates (Analtech, Newark, Del.) and was visualized by fluorescence quenching under UV light. Biotage SPl Flash Chromatography Purification System (Charlotte, N.C.) (Biotage SNAP Cartridge, silica, 50 g & 100 g) was used to purify the compounds. ¹H NMR and ¹³C NMR spectra were recorded on a Bruker Ascend 400 (400 MHz) (Billerica, Mass.) spectrometer. Chemical shifts for ¹H NMR were reported in parts per million (ppm) downfield from tetramethylsilane (S) as the internal standard in deuterated solvent and coupling constants (J) are in Hertz (Hz). The following abbreviations are used for spin multiplicity: s=singlet, d=doublet, t=triplet, q=quartet, quin=quintet, dd=doublet of doublets, dt=doublet of triplets, qd=quartet of doublets, dquin=doublet of quintets, m=multiplet, and br s=broad singlet. Low-resolution mass spectra (MS) were acquired using a Brucker ESQUIRE electrospray/ion trap instrument in the positive and negative modes. High resolution mass spectrometer (HRMS) data were acquired on a Waters Xevo G2-S QTOF (Milford, Mass.) system equipped with an Acquity I-class UPLC system.

### Example 1

### Synthesis of Pyrazolylpropanamide Compounds

A series of pyrazol-1-yl-propanamide compounds with varying mono-substituents of the pyrazole B-ring (Series I), variations of the aromatic A-ring (Series II), varying the di-substituents of the pyrazole B-ring (Series III), or modifications of the linkage moiety (Series IV) were synthesized, as shown in Table 1.

TABLE 1
Structures of Pyrazol-1-YI-Propanamide AR Antagonists
Series I. Monosubstitution of the Pyrazole Moiety (B-ring)

| ID | Structure |
|---|---|
| 16a | 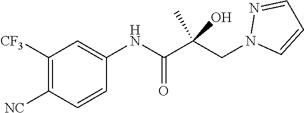 [SMILES: C[C@](O)(Cn1cccn1)C(=O)Nc1ccc(C#N)c(C(F)(F)F)c1] |
| 10 (UT- 034) | 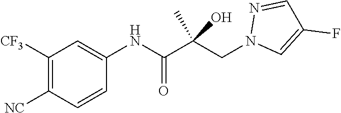 [SMILES: C[C@](O)(Cn1cc(F)cn1)C(=O)Nc1ccc(C#N)c(C(F)(F)F)c1] |
| 16b | 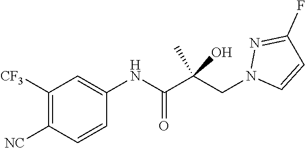 [SMILES: C[C@](O)(Cn1ccc(F)n1)C(=O)Nc1ccc(C#N)c(C(F)(F)F)c1] |
| 16c | 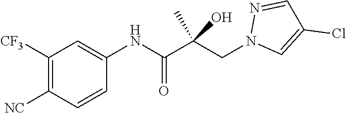 [SMILES: C[C@](O)(Cn1cc(Cl)cn1)C(=O)Nc1ccc(C#N)c(C(F)(F)F)c1] |
| 16d | 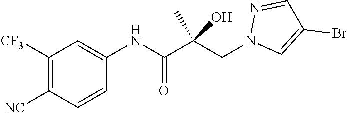 [SMILES: C[C@](O)(Cn1cc(Br)cn1)C(=O)Nc1ccc(C#N)c(C(F)(F)F)c1] |
| 16e | 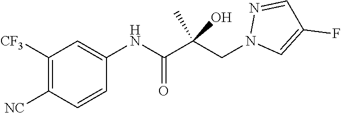 [SMILES: C[C@](O)(Cn1cc(F)cn1)C(=O)Nc1ccc(C#N)c(C(F)(F)F)c1] |
| 16f | 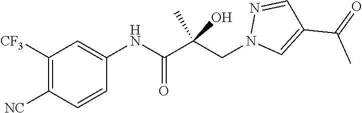 [SMILES: CC(=O)c1cnn(C[C@](C)(O)C(=O)Nc2ccc(C#N)c(C(F)(F)F)c2)c1] |
| 16g | 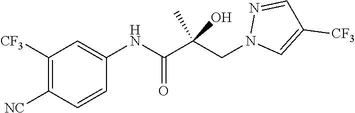 [SMILES: C[C@](O)(Cn1cc(C(F)(F)F)cn1)C(=O)Nc1ccc(C#N)c(C(F)(F)F)c1] |
| 16h | 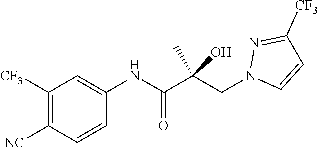 [SMILES: C[C@](O)(Cn1ccc(C(F)(F)F)n1)C(=O)Nc1ccc(C#N)c(C(F)(F)F)c1] |
| 16i | 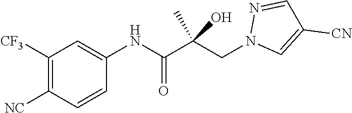 [SMILES: C[C@](O)(Cn1cc(C#N)cn1)C(=O)Nc1ccc(C#N)c(C(F)(F)F)c1] |
| 16j | 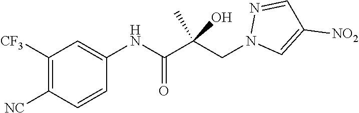 [SMILES: C[C@](O)(Cn1cc([N+](=O)[O-])cn1)C(=O)Nc1ccc(C#N)c(C(F)(F)F)c1] |
| 16k | 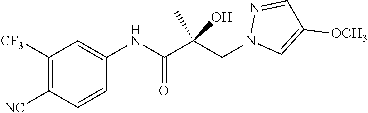 [SMILES: COc1cnn(C[C@](C)(O)C(=O)Nc2ccc(C#N)c(C(F)(F)F)c2)c1] |
| 16l | 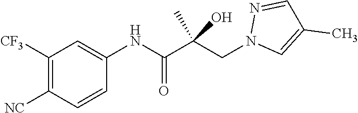 [SMILES: Cc1cnn(C[C@](C)(O)C(=O)Nc2ccc(C#N)c(C(F)(F)F)c2)c1] |
| 16m | 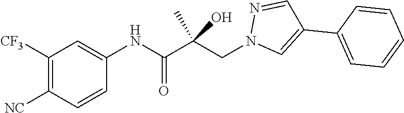 [SMILES: C[C@](O)(Cn1cc(-c2ccccc2)cn1)C(=O)Nc1ccc(C#N)c(C(F)(F)F)c1] |
| 16n | 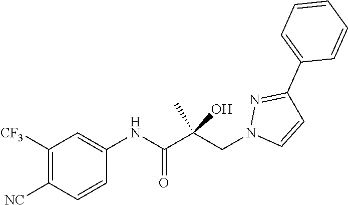 [SMILES: C[C@](O)(Cn1ccc(-c2ccccc2)n1)C(=O)Nc1ccc(C#N)c(C(F)(F)F)c1] |
| 16o | 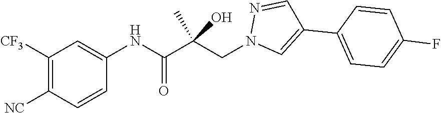 [SMILES: C[C@](O)(Cn1cc(-c2ccc(F)cc2)cn1)C(=O)Nc1ccc(C#N)c(C(F)(F)F)c1] |
| 16p | 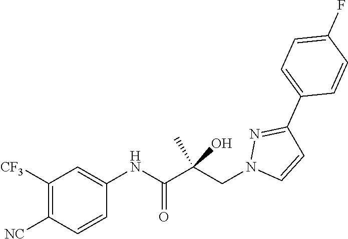 [SMILES: C[C@](O)(Cn1ccc(-c2ccc(F)cc2)n1)C(=O)Nc1ccc(C#N)c(C(F)(F)F)c1] |
| 16q | 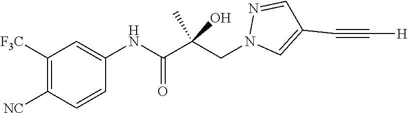 [SMILES: C#Cc1cnn(C[C@](C)(O)C(=O)Nc2ccc(C#N)c(C(F)(F)F)c2)c1] |
| 16r | 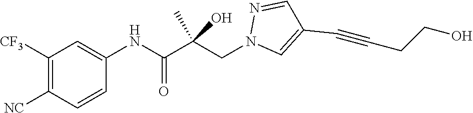 [SMILES: C[C@](O)(Cn1cc(C#CCCO)cn1)C(=O)Nc1ccc(C#N)c(C(F)(F)F)c1] |
| 16s | 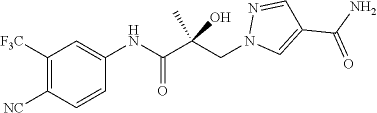 [SMILES: C[C@](O)(Cn1cc(C(N)=O)cn1)C(=O)Nc1ccc(C#N)c(C(F)(F)F)c1] |
| 16t | 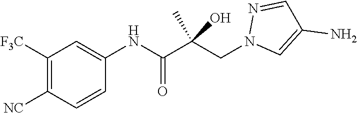 [SMILES: C[C@](O)(Cn1cc(N)cn1)C(=O)Nc1ccc(C#N)c(C(F)(F)F)c1] |
| 16u | 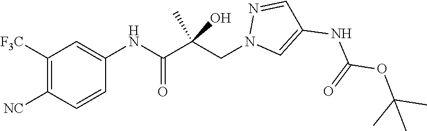 [SMILES: CC(C)(C)OC(=O)Nc1cnn(C[C@](C)(O)C(=O)Nc2ccc(C#N)c(C(F)(F)F)c2)c1] |
| 16v | 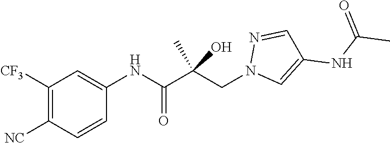 [SMILES: CC(=O)Nc1cnn(C[C@](C)(O)C(=O)Nc2ccc(C#N)c(C(F)(F)F)c2)c1] |
| 16w | 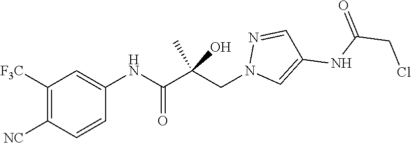 [SMILES: C[C@](O)(Cn1cc(NC(=O)CCl)cn1)C(=O)Nc1ccc(C#N)c(C(F)(F)F)c1] |
| 16x | 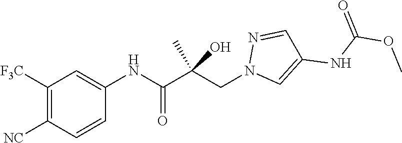 [SMILES: COC(=O)Nc1cnn(C[C@](C)(O)C(=O)Nc2ccc(C#N)c(C(F)(F)F)c2)c1] |

Series II. Variations of the Aromatic A-Ring

| | |
|---|---|
| 21a | 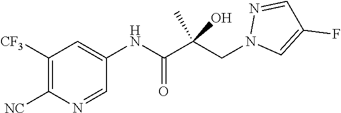 [SMILES: C[C@](O)(Cn1cc(F)cn1)C(=O)Nc1cnc(C#N)c(C(F)(F)F)c1] |
| 21b | 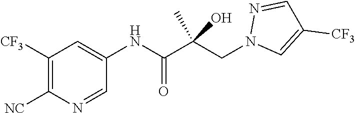 [SMILES: C[C@](O)(Cn1cc(C(F)(F)F)cn1)C(=O)Nc1cnc(C#N)c(C(F)(F)F)c1] |
| 21c | 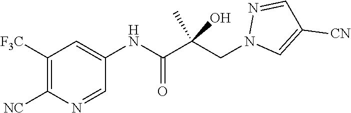 [SMILES: C[C@](O)(Cn1cc(C#N)cn1)C(=O)Nc1cnc(C#N)c(C(F)(F)F)c1] |
| 21d | 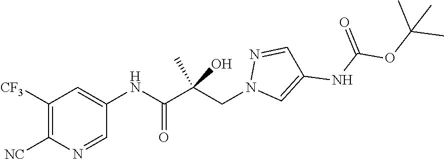 [SMILES: CC(C)(C)OC(=O)Nc1cnn(C[C@](C)(O)C(=O)Nc2cnc(C#N)c(C(F)(F)F)c2)c1] |
| 21e | 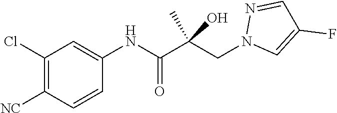 [SMILES: C[C@](O)(Cn1cc(F)cn1)C(=O)Nc1ccc(C#N)c(Cl)c1] |
| 21f | 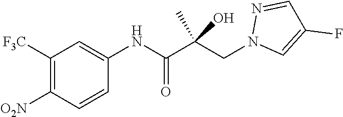 [SMILES: C[C@](O)(Cn1cc(F)cn1)C(=O)Nc1ccc([N+](=O)[O-])c(C(F)(F)F)c1] |
| 21g | 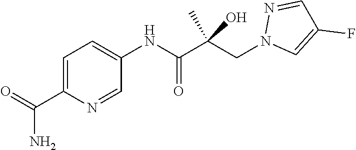 [SMILES: C[C@](O)(Cn1cc(F)cn1)C(=O)Nc1ccc(C(N)=O)nc1] |
| 21h | 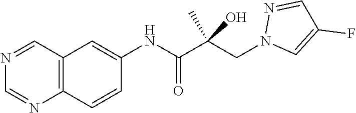 [SMILES: C[C@](O)(Cn1cc(F)cn1)C(=O)Nc1ccc2ncncc2c1] |
| 21i | 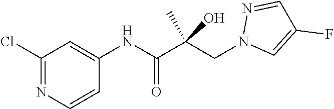 [SMILES: C[C@](O)(Cn1cc(F)cn1)C(=O)Nc1ccnc(Cl)c1] |
| 21j | 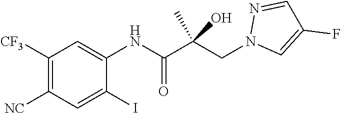 [SMILES: C[C@](O)(Cn1cc(F)cn1)C(=O)Nc1cc(C(F)(F)F)c(C#N)cc1I] |

Series III. Disubstitution of the Pyrazole B-ring

| | |
|---|---|
| 26a | 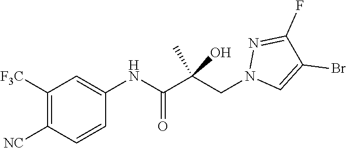 [SMILES: C[C@](O)(Cn1cc(Br)c(F)n1)C(=O)Nc1ccc(C#N)c(C(F)(F)F)c1] |
| 26b | 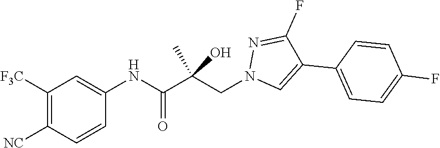 [SMILES: C[C@](O)(Cn1cc(-c2ccc(F)cc2)c(F)n1)C(=O)Nc1ccc(C#N)c(C(F)(F)F)c1] |
| 26c | 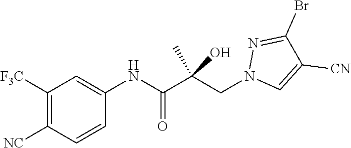 [SMILES: C[C@](O)(Cn1cc(C#N)c(Br)n1)C(=O)Nc1ccc(C#N)c(C(F)(F)F)c1] |
| 26d | 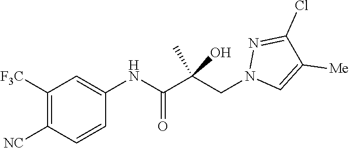 [SMILES: Cc1cn(C[C@](C)(O)C(=O)Nc2ccc(C#N)c(C(F)(F)F)c2)nc1Cl] |
| 26e | 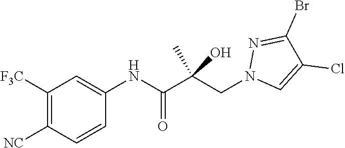 [SMILES: C[C@](O)(Cn1cc(Cl)c(Br)n1)C(=O)Nc1ccc(C#N)c(C(F)(F)F)c1] |
| 26f | 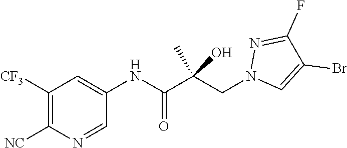 [SMILES: C[C@](O)(Cn1cc(Br)c(F)n1)C(=O)Nc1cnc(C#N)c(C(F)(F)F)c1] |
| 26g | 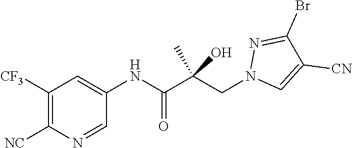 [SMILES: C[C@](O)(Cn1cc(C#N)c(Br)n1)C(=O)Nc1cnc(C#N)c(C(F)(F)F)c1] |
| 26h | 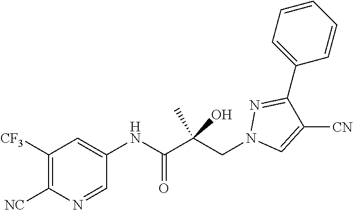 [SMILES: C[C@](O)(Cn1cc(C#N)c(-c2ccccc2)n1)C(=O)Nc1cnc(C#N)c(C(F)(F)F)c1] |

Series IV. Modification of Linkage Moiety

| | |
|---|---|
| 29a (R- isomer of 10) | 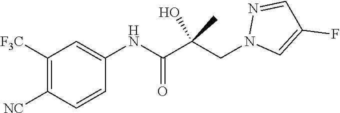 [SMILES: C[C@@](O)(Cn1cc(F)cn1)C(=O)Nc1ccc(C#N)c(C(F)(F)F)c1] |
| 29b | 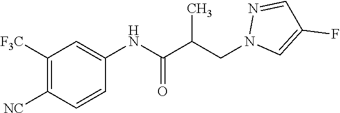 [SMILES: CC(Cn1cc(F)cn1)C(=O)Nc1ccc(C#N)c(C(F)(F)F)c1] |
| 29c | 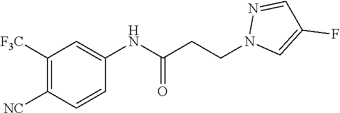 [SMILES: N#Cc1ccc(NC(=O)CCn2cc(F)cn2)cc1C(F)(F)F] |
| 29d | 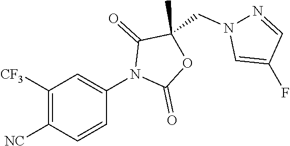 [SMILES: C[C@@]1(Cn2cc(F)cn2)OC(=O)N(c2ccc(C#N)c(C(F)(F)F)c2)C1=O] |
| 29e | 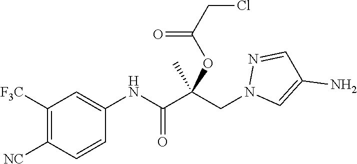 [SMILES: C[C@@](Cn1cc(N)cn1)(OC(=O)CCl)C(=O)Nc1ccc(C#N)c(C(F)(F)F)c1] |
| 29f | 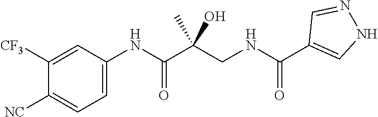 [SMILES: C[C@](O)(CNC(=O)c1cn[nH]c1)C(=O)Nc1ccc(C#N)c(C(F)(F)F)c1] |

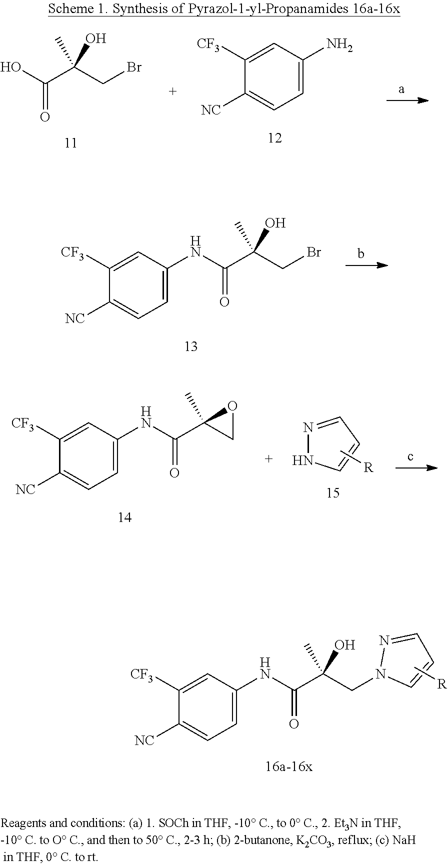

### Synthesis of Compound 13

(R)-3-Bromo-2-hydroxy-2-methylpropanoic acid 11 (5.00 g, 27 mmol) was dissolved in THF (27 mL, 5.4 vol) in an EasyMax 100 mL reactor. Agitation was set to 400 rpm and the solution cooled to 2.5° C. Thionyl chloride (2.39 mL, 1.20 equiv, 0.48 vol) was slowly added to the reaction mixture over 30 min while maintaining the reaction temperature below 12° C. The reaction mixture was stirred for 1.5 h. The reaction was cooled to −5° C. Triethylamine (5.0 mL, 1.30 equiv, 1 vol) was slowly added to the reaction mixture, keeping the temperature below 12° C. 4-Amino-2-(trifluoromethyl)benzonitrile 12 (4.85 g, 0.95 equiv, 0.97 wt) and THF (3.37 mL, 0.67 vol) were then charged to the batch. The batch was then heated to 50±5° C. and agitated for two hours. The batch was then cooled to 20±5° C. followed by the addition of water (14.7 mL, 2.9 vol) and toluene (20.2 mL, 4.0 vol). After brief agitation the layers were separated. The organic layer was then washed with water (14.7 mL, 2.9 vol). The batch was then concentrated to 5±0.5 volumes (4±0.5 wt) while maintaining the batch temperature below 50° C., followed by the addition of toluene (30 mL, 6 vol). The batch was then distilled to 5±0.5 volumes (4±0.5 wt) and the batch temperature reduced to 2.5±2.5° C. The batch was then filtered, and the filter cake washed with toluene twice (8.5 mL each, 1.7 vol each). The batch was then dried under 25-30 inches vacuum to provide (R)-3-bromo-N-(4-cyano-3-(trifluoromethyl)phenyl)-2-hydroxy-2-methylpropanamide 13.

### Synthesis of Compound 14

To a solution of (R)-3-bromo-N-(4-cyano-3-(trifluoromethyl)phenyl)-2-hydroxy-2-methylpropanamide 13 (5.00 g, 0.018504 mol) in 25 mL of 2-butanone, was added potassium carbonate (3.836 g, 0.027756 mol). The resulting reaction mixture was heated at reflux for 2 h under argon atmosphere. After the end of the reaction was established by TLC, the reaction was cooled to room temperature (rt), filtered through a pad of Celite, and rinsed the pad of Celite with 15 mL of 2-butanone. The filtrate was concentrated under vacuum, and dried under 25-30 inches vacuum to provide (S)—N-(4-cyano-3-(trifluoromethyl)phenyl)-2-methyloxirane-2-carboxamide 14.

### General Procedure A

### Synthesis of 16(a-y), 21(a-k), 26(a-h) as well as 29(a-p), Using 10 (UT-34) as an Example

To a solution of 4-fluoro-pyrazole (0.10 g, 0.00116 mol), or general pyrazole 15, in anhydrous THF (10 mL) which was cooled in an ice water bath under an argon atmosphere, was added sodium hydride (60% dispersion in oil, 0.12 g, 0.00291 mol). After addition, the resulting mixture was stirred for three hours. (R)-3-Bromo-N-(4-cyano-3-(trifluoromethyl)phenyl)-2-hydroxy-2-methylpropanamide 13 (0.41 g, 0.00116 mol) or (S)—N-(4-cyano-3-(trifluoromethyl)phenyl)-2-methyloxirane-2-carboxamide 14 (0.313 g, 0.00116 mol) was added to above solution, and the resulting reaction mixture was allowed to stir overnight at rt under argon. The reaction was quenched with water and extracted with ethyl acetate. The organic layer was washed with brine, dried with MgSO₄, filtered, and concentrated under vacuum. The product was purified by a silica gel column using ethyl acetate and hexanes (1:1) as eluent to afford 0.13 g of 10 as white solid.

### (S)—N-(4-Cyano-3-(trifluoromethyl)phenyl)-2-hydroxy-2-methyl-3-(1H-pyrazol-1-yl)propanamide (16a)

Compound 16a was prepared following General Procedure A. The crude product was purified by a silica gel column using ethyl acetate and hexanes (2:1) as eluent to afford 0.52 g of the titled compound as white solid. Yield=52%. 1H NMR (400 MHz, DMSO-d₆) δ 10.39 (s, 1H, NH), 8.48 (d, J=2.0 Hz, 1H, ArH), 8.22 (dd, J=8.2 Hz, J=2.0 Hz, 1H, ArH), 8.08 (d, J=8.2 Hz, 1H, ArH), 7.66-7.65 (m, 1H, Pyrazole-H), 7.39-7.38 (m, 1H, Pyrazole-H), 6.28 (s, 1H, OH), 6.25-6.23 (m, 1H, Pyrazole-H), 4.50 (d, J=13.6 Hz, 1H, CH), 4.29 (d, J=13.6 Hz, 1H, CH), 1.35 (s, 3H, CH₃). HRMS [C₁₅H₁₄F₃N₄O₂+]: calcd 339.1099, found 339.1105 [M+H]+.

### (S)—N-(4-Cyano-3-(trifluoromethyl)phenyl)-3-(4-fluoro-1H-pyrazol-1-yl)-2-hydroxy-2-methylpropanamide (10 (UT-34))

Compound 10 was prepared following General Procedure A per Scheme 1. The crude product was purified by a silica gel column using ethyl acetate and hexanes (1:1) as eluent to afford 0.13 g of the titled compound as white solid. Yield=32%. ¹H NMR (400 MHz, DMSO-d₆) δ 10.39 (s, 1H, NH), 8.47 (d, J=1.6 Hz, 1H, ArH), 8.24 (dd, J=8.4 Hz, J=2.0 Hz, 1H, ArH), 8.10 (d, J=8.4 Hz, 1H, ArH), 7.73 (d, J=4.4 Hz, 1H, Pyrazole-H), 7.41 (d, J=4.4 Hz, 1H, Pyrazole-H), 6.31 (s, 1H, OH), 4.38 (d, J=14.0 Hz, 1H, CH), 4.21 (d, J=14.0 Hz, 1H, CH), 1.34 (s, 3H, CH₃). HRMS [C₁₅H₁₃F4N₄O₂+]: calcd 357.0975, found 357.0966[M+H]⁺.

### (S)—N-(4-Cyano-3-(trifluoromethyl)phenyl)-3-(3-fluoro-1H-pyrazol-1-yl)-2-hydroxy-2-methylpropanamide (16b)

Compound 16b was prepared following General Procedure A per Scheme 1. The crude product was purified by a silica gel column using ethyl acetate and hexanes (2:1) as eluent to afford 0.36 g of the titled compound as white needles. Yield=44%. ¹H NMR (400 MHz, DMSOd₆) δ 10.39 (s, 1H, NH), 8.47 (d, J=2.0 Hz, 1H, ArH), 8.24 (dd, J=8.8 Hz, J=2.0 Hz, 1H, ArH), 8.11 (d, J=8.8 Hz, 1H, ArH), 7.55 (t, J=3.0 Hz, 1H, Pyrazole-H), 6.29 (s, 1H, OH), 5.93-5.91 (m, 1H, Pyrazole-H), 4.34 (d, J=13.6 Hz, 1H, CH), 4.15 (d, J=13.6 Hz, 1H, CH), 1.36 (s, 3H, CH₃). HRMS [C₁₅H₁₃F₄N₄O₂+]: calcd 357.0975, found 357.0985[M+H]⁺.

### (S)-3-(4-Chloro-1H-pyrazol-1-yl)-N-(4-cyano-3-(trifluoromethyl)phenyl)-2-hydroxy-2-methylpropanamide (16c)

Compound 16c was prepared following General Procedure A per Scheme 1. The crude product was purified by a silica gel column using DCM and ethyl acetate (19:1) as eluent to afford 0.30 g of the titled compound as white solid. Yield=55%. ¹H NMR (400 MHz, DMSO-d₆) δ 10.38 (s, 1H, NH), 8.46 (s, 1H, ArH), 8.23 (d, J=8.6 Hz, J=1.2 Hz, 1H, ArH), 8.10 (d, J=8.6 Hz, 1H, ArH), 7.83 (s, 1H, Pyrazole-H), 7.47 (s, 1H, Pyrazole-H), 6.34 (s, 1H, OH), 4.45 (d, J=14.0 Hz, 1H, CH), 4.27 (d, J=14.0 Hz, 1H, CH), 1.36 (s, 3H, CH₃). HRMS [C₁₅H₁₃ClF₃N₄O₂+]: calcd 373.0679, found 373.0678[M+H]⁺. Purity: 97.69% (HPLC).

### (S)-3-(4-Bromo-1H-pyrazol-1-yl)-N-(4-cyano-3-(trifluoromethyl)phenyl)-2-hydroxy-2-methylpropanamide (16d)

Compound 16d was prepared following General Procedure A per Scheme 1. The crude product was purified by a silica gel column using DCM and ethyl acetate (19:1) as eluent to afford 0.47 g of the titled compound as white form. Yield=79.6%. ¹H NMR (400 MHz, CDCl₃) δ 9.08 (s, 1H, NH), 8.00 (d, J=2.0 Hz, 1H, ArH), 7.87 (dd, J=8.4 Hz, J=2.0 Hz, 1H, ArH), 7.79 (d, J=8.4 Hz, 1H, ArH), 7.49 (s, 1H, Pyrazole-H), 7.47 (s, 1H, Pyrazole-H), 5.92 (s, 1H, OH), 4. (d, J=14.0 Hz, 1H, CH), 4.24 (d, J=14.0 Hz, 1H, CH), 1.47 (s, 3H, CH₃). HRMS [C₁₅H₁₃BrF₃N₄O₂+]: calcd 417.0174, found 417.0167[M+H]⁺. Purity: 99.53% (HPLC).

### (S)—N-(4-Cyano-3-(trifluoromethyl)phenyl)-2-hydroxy-3-(4-iodo-1H-pyrazol-1-yl)-2-methylpropanamide (16e)

Compound 16e was prepared following General Procedure A per Scheme 1. The crude product was purified by a silica gel column using DCM and ethyl acetate (19:1) as eluent to afford 0.25 g of the titled compound as off-white solid. Yield=52%. ¹H NMR (400 MHz, DMSO-d₆) δ 10.36 (s, 1H, NH), 8.45 (s, 1H, ArH), 8.23 (d, J=8.8 Hz, J=1.2 Hz, 1H, ArH), 8.10 (d, J=8.8 Hz, 1H, ArH), 7.78 (s, 1H, Pyrazole-H), 7.46 (s, 1H, Pyrazole-H), 6.31 (s, 1H, OH), 4.48 (d, J=14.0 Hz, 1H, CH), 4.31 (d, J=14.0 Hz, 1H, CH), 1.35 (s, 3H, CH₃). HRMS [C₁₅H₁₃F₃₁N₄O₂+]: calcd 465.0035, found 465.0045[M+H]⁺.

### (S)-3-(4-Acetyl-1H-pyrazol-1-yl)-N-(4-cyano-3-(trifluoromethyl)phenyl)-2-hydroxy-2-methylpropanamide (16f)

Compound 16f was prepared following General Procedure A per Scheme 1.

The product was purified by a silica gel column using DCM and ethyl acetate (19:1) as eluent to afford 70 mg of the titled compound as yellowish solid. Yield=20%. ¹H NMR (400 MHz, DMSO-d₆) δ 10.37 (s, 1H, NH), 8.45 (d, J=1.2 Hz, 1H, ArH), 8.25 (s, 1H, Pyrazole-H), 8.23 (d, J=8.2 Hz, J=1.2 Hz, 1H, ArH), 8.10 (d, J=8.2 Hz, 1H, ArH), 7.86 (s, 1H, PyrazoleH), 6.37 (s, 1H, OH), 4.50 (d, J=14.0 Hz, 1H, CH), 4.33 (d, J=14.0 Hz, 1H, CH), 2.34 (s, 3H, CH₃), 1.39 (s, 3H, CH₃). HRMS [CnH₁₆F₃N₄O₃+]: calcd 381.1175, found 381.1178[M+H]⁺. Purity: 95.66% (HPLC).

### (S)—N-(4-Cyano-3-(trifluoromethyl)phenyl)-2-hydroxy-2-methyl-3-(4-(trifluoromethyl)-1H-pyrazol-1-yl)propanamide (16g)

Compound 16g was prepared following General Procedure A per Scheme 1. The product was purified by a silica gel column using DCM and ethyl acetate (19:1) as eluent to afford 0.30 g of the titled compound as white foam. Yield=50%. ¹H NMR (400 MHz, DMSO-d₆) δ 10.38 (s, 1H, NH), 8.45 (d, J=2.0 Hz, 1H, ArH), 8.25-8.22 (m, 2H, ArH & Pyrazole-H), 8.11 (d, J=8.2 Hz, 1H, ArH), 7.82 (s, 1H, Pyrazole-H), 6.39 (s, 1H, OH), 4.55 (d, J=14.0 Hz, 1H, CH), 4.37 (d, J=14.0 Hz, 1H, CH), 1.40 (s, 3H, CH₃). HRMS [C₁₆H₁₃F₆N₄O₂]⁺: calcd 407.0943, found 407.0945 [M+H]⁺.

### (S)—N-(4-Cyano-3-(trifluoromethyl)phenyl)-2-hydroxy-2-methyl-3-(3-(trifluoromethyl)-1H-pyrazol-1-yl)propanamide (16h)

Compound 16h was prepared following General Procedure A per Scheme 1. The product was purified by a silica gel column using ethyl acetate and hexanes (2:1) as eluent to afford 0.31 g of the titled compound as white solid. Yield=50%. ¹H NMR (400 MHz, DMSOd₆) δ 10.31 (s, 1H, NH), 8.42 (d, J=2.0 Hz, 1H, ArH), 8.18 (dd, J=8.8 Hz, J=2.0 Hz, 1H, ArH), 8.09 (d, J=8.8 Hz, 1H, ArH), 7.84-7.83 (m, 1H, Pyrazole-H), 6.67 (d, J=2.4 Hz, 1H, pyrazole-H), 6.41 (s, 1H, OH), 4.56 (d, J=14.0 Hz, 1H, CH), 4.38 (d, J=14.0 Hz, 1H, CH), 1.40 (s, 3H, CH₃). HRMS [C₁₆H₁₃F₆N₄O₂+]: calcd 407.0943, found 407.0945 [M+H]⁺.

### (S)-3-(4-Cyano-1H-pyrazol-1-yl)-N-(4-cyano-3-(trifluoromethyl)phenyl)-2-hydroxy-2-methylpropanamide (16i)

Compound 16i was prepared following General Procedure A per Scheme 1. The product was purified by a silica gel column using hexanes and ethyl acetate (1:1 to 1:2) as eluent to afford 0.18 g of the titled compound as white solid. Yield=46%. ¹H NMR (400 MHz, DMSOd₆) δ 10.35 (s, 1H, NH), 8.45 (d, J=1.2 Hz, 1H, ArH), 8.43 (s, 1H, Pyrazole-H), 8.22 (d, J=8.8 Hz, J=1.2 Hz, 1H, ArH), 8.10 (d, J=8.8 Hz, 1H, ArH), 7.98 (s, 1H, Pyrazole-H), 6.41 (s, 1H, OH), 4.45 (d, J=14.0 Hz, 1H, CH), 4.36 (d, J=14.0 Hz, 1H, CH), 1.38 (s, 3H, CH₃). HRMS [C₁₆H₁₃F₃N₅O₂+]: calcd 364.1021, found 364.1016[M+H]⁺. Purity: 98.48% (HPLC).

### (S)—N-(4-Cyano-3-(trifluoromethyl)phenyl)-2-hydroxy-2-methyl-3-(4-nitro-1H-pyrazol-1-yl)propanamide (16j)

Compound 16j was prepared following General Procedure A per Scheme 1. The product was purified by a silica gel column using hexanes and ethyl acetate (1:1) as eluent to afford 0.15 g of the titled compound as off-white solid. Yield=44%. ¹H NMR (400 MHz, DMSO-d₆) δ 10.36 (s, 1H, NH), 8.69 (s, 1H, Pyrazole-H), 8.45 (d, J=1.2 Hz, 1H, ArH), 8.23 (d, J=8.8 Hz, J=1.2 Hz, 1H, ArH), 8.19 (s, 1H, Pyrazole-H), 8.11 (d, J=8.8 Hz, 1H, ArH), 6.47 (s, 1H, OH), 4.56 (d, J=14.0 Hz, 1H, CH), 4.38 (d, J=14.0 Hz, 1H, CH), 1.41 (s, 3H, CH₃). HRMS [C₁₅H₁₃F₃N₅O₄+]: calcd 384.0920, found 384.0932[M+H]⁺. Purity: 99.58% (HPLC).

### (S)—N-(4-Cyano-3-(trifluoromethyl)phenyl)-2-hydroxy-3-(4-methoxy-1H-pyrazol-1-yl)-2-methylpropanamide (16k)

Compound 16k was prepared following General Procedure A per Scheme 1. The product was purified by a silica gel column using DCM and ethyl acetate (9:1) as eluent to afford 0.30 g of the titled compound as white solid. Yield=60%. ¹H NMR (400 MHz, DMSO-d₆) δ 10.38 (s, 1H, NH), 8.46 (d, J=2.0 Hz, 1H, ArH), 8.24 (dd, J=8.2 Hz, J=2.0 Hz, 1H, ArH), 8.10 (d, J=8.2 Hz, 1H, ArH), 7.35 (d, J=0.8 Hz, 1H, Pyrazole-H), 7.15 (d, J=0.8 Hz, 1H, Pyrazole-H), 6.25 (s, 1H, OH), 4.35 (d, J=14.0 Hz, 1H, CH), 4.18 (d, J=14.0 Hz, 1H, CH), 3.61 (s, 3H, CH₃), 1.36 (s, 3H, CH₃). HRMS [C₁₆H₁₆F₃N₄O₃⁺]: calcd 369.1175, found 369.1182[M+H]⁺. Purity: 99.28% (HPLC).

### (S)—N-(4-Cyano-3-(trifluoromethyl)phenyl)-2-hydroxy-2-methyl-3-(4-methyl-1H-pyrazol-1-yl)propanamide (16l)

Compound 16l was prepared following General Procedure A per Scheme 1. The product was purified by a silica gel column using DCM and ethyl acetate (19:1) as eluent to afford 0.28 g of the titled compound as white solid. Yield=66%. ¹H NMR (400 MHz, DMSO-d₆) δ 10.38 (s, 1H, NH), 8.46 (d, J=2.0 Hz, 1H, ArH), 8.23 (dd, J=8.8 Hz, J=2.0 Hz, 1H, ArH), 8.10 (d, J=8.8 Hz, 1H, ArH), 7.41 (s, 1H, Pyrazole-H), 7.17 (s, 1H, Pyrazole-H), 6.24 (s, 1H, OH), 4.40 (d, J=14.0 Hz, 1H, CH), 4.22 (d, J=14.0 Hz, 1H, CH), 1.97 (s, 3H, CH₃), 1.36 (s, 3H, CH₃). HRMS [C₁₆H₁₆F₃N₄O₂+]: calcd 353.1225, found 353.1232[M+H]⁺. Purity: 99.75% (HPLC). (S)—N-(4-Cyano-3-(trifluoromethyl)phenyl)-2-hydroxy-2-methyl-3-(4-phenyl-1H-pyrazol-1-yl)propanamide (16m)

Compound 16m was prepared following General Procedure A per Scheme 1. The product was purified by a silica gel column using ethyl acetate and hexanes (1:2) as eluent to afford 0.90 g of the titled compound as white needles. Yield=68.5%. ¹H NMR (400 MHz, DMSO-d₆) δ 10.40 (s, 1H, NH), 8.46 (d, J=2.0 Hz, 1H, ArH), 8.24 (dd, J=8.4 Hz, J=2.0 Hz, 1H, ArH), 8.09 (d, J=8.4 Hz, 1H, ArH), 8.05 (s, 1H, Pyrazole-H), 7.82 (s, 1H, Pyrazole-H), 7.52-7.45 (m, 2H, ArH), 7.35-7.31 (m, 2H, ArH), 7.20-7.16 (m, 1H, ArH), 6.33 (s, 1H, OH), 4.50 (d, J=14.0 Hz, 1H, CH), 4.30 (d, J=14.0 Hz, 1H, CH), 1.40 (s, 3H, CH₃). HRMS [C₂₁H₁₅F₃N₄O₂+]: calcd 415.1382, found 415.1391[M+H]⁺.

### (S)—N-(4-Cyano-3-(trifluoromethyl)phenyl)-2-hydroxy-2-methyl-3-(3-phenyl-1H-pyrazol-1-yl)propanamide (16n)

Compound 16n was prepared following General Procedure A per Scheme 1. The product was purified by a silica gel column using ethyl acetate and hexanes (1:3 to 1:2) as eluent to afford 0.60 g of the titled compound as white needles. Yield=41.7%. 1H NMR (400 MHz, DMSO-d₆) δ 10.33 (s, 1H, NH), 8.48 (d, J=2.0 Hz, 1H, ArH), 8.22 (dd, J=8.2 Hz, J=2.0 Hz, 1H, ArH), 8.05 (d, J=8.2 Hz, 1H, ArH), 7.69 (d, J=2.0 Hz, 1H, ArH), 7.60-7.57 (m, 2H, ArH), 7.28-7.21 (m, 3H, ArH), 6.66 (d, J=3.0 Hz, 1H, ArH), 6.31 (s, 1H, OH), 4.52 (d, J=14.6 Hz, 1H, CH), 4.32 (d, J=14.6 Hz, 1H, CH), 1.43 (s, 3H, CH₃). Mass (ESI, Positive): [C₂₁H₁₅F₃N₄O₂⁺]: calcd 415.1382, found 514.1423 [M+H]⁺.

### (S)—N-(4-Cyano-3-(trifluoromethyl)phenyl)-3-(4-(4-fluorophenyl)-1H-pyrazol-1-yl)-2-hydroxy-2-methylpropanamide (16o)

Compound 160 was prepared following General Procedure A per Scheme 1. The product was purified by a silica gel column using DCM and ethyl acetate (19:1) as eluent to afford 0.33 g of the titled compound as white solid. Yield=62%. 1H NMR (400 MHz, DMSO-d₆) δ 10.29 (s, 1H, NH), 8.41 (s, 1H, ArH), 8.21 (d, J=8.8 Hz, 1H, ArH), 8.05 (d, J=8.8 Hz, 1H, ArH), 7.68 (s, 1H, Pyrazole-H), 7.61 (t, J=6.4 Hz, 2H, ArH), 7.08 (t, J=8.4 Hz, 2H, ArH), 6.65 (s, 1H, Pyrazole-H), 6.30 (s, 1H, OH), 4.51 (d, J=14.0 Hz, 1H, CH), 4.31 (d, J=14.0 Hz, 1H, CH), 1.42 (s, 3H, CH₃). HRMS [C₂₁H₁₁F₄N₄O₂⁺]: calcd 433.1288, found 433.1291[M+H]⁺. Purity: 96.01% (HPLC).

### (S)—N-(4-Cyano-3-(trifluoromethyl)phenyl)-3-(3-(4-fluorophenyl)-1H-pyrazol-1-yl)-2-hydroxy-2-methylpropanamide (16p)

Compound 16p was prepared following General Procedure A per Scheme 1. The product was purified by a silica gel column using DCM and ethyl acetate (19:1) as eluent to afford 0.27 g of the titled compound as white solid. Yield=43%. 1H NMR (400 MHz, DMSO-d₆) δ 10.29 (s, 1H, NH), 8.41 (s, 1H, ArH), 8.21 (d, J=8.8 Hz, 1H, ArH), 8.05 (d, J=8.8 Hz, 1H, ArH), 7.69 (s, 1H, Pyrazole-H), 7.61 (t, J=6.4 Hz, 2H, ArH), 7.08 (t, J=8.4 Hz, 2H, ArH), 6.65 (s, 1H, Pyrazole-H), 6.30 (s, 1H, OH), 4.51 (d, J=14.0 Hz, 1H, CH), 4.31 (d, J=14.0 Hz, 1H, CH), 1.42 (s, 3H, CH₃). Mass (ESI, Negative): 431.12 [M−Hr. HRMS [C₂₁H₁₁F₄N₄O₂⁺]: calcd 433.1288, found 433.1290[M+H]⁺.

### (S)—N-(4-Cyano-3-(trifluoromethyl)phenyl)-3-(4-ethynyl-1H-pyrazol-1-yl)-2-hydroxy-2-methylpropanamide (16q)

Compound 16q was prepared following General Procedure A per Scheme 1. The product was purified by a silica gel column using DCM and ethyl acetate (95:5) as eluent to afford 0.37 g of the titled compound as white foam. Yield=62.7%. ¹H NMR (400 MHz, DMSO-d₆) δ 10.40 (s, 1H, NH), 8.47 (s, 1H, ArH), 8.24 (d, J=8.8 Hz, 1H, ArH), 8.11 (d, J=8.8 Hz, 1H, ArH), 7.91 (s, 1H, Pyrazole-H), 7.57 (s, 1H, Pyrazole-H), 6.35 (s, 1H, OH), 4.46 (d, J=14.4 Hz, 1H, CH), 4.29 (d, J=14.4 Hz, 1H, CH), 4.00 (s, 1H, CH), 1.35 (s, 3H, CH₃). HRMS [C₁₁H₁₄F₃N₄O₂⁺]: calcd 363.1069, found 363.1026 [M+H]⁺. Purity: 99.55% (HPLC).

### (S)—N-(4-Cyano-3-(trifluoromethyl)phenyl)-2-hydroxy-3-(4-(4-hydroxybut-1-yn-1-yl)-1H-pyrazol-1-yl)-2-methylpropanamide (16r)

Compound 16r was prepared following General Procedure A per Scheme 1. The product was purified by a silica gel column using DCM and methanol (95:5) as eluent to afford 0.477 g of the titled compound as yellowish solid. Yield=20%. ¹H NMR (400 MHz, DMSO-d₆) δ 12.99 (brs, 1H), 10.47 (s, 1H, NH), 8.55 (s, 1H, ArH), 8.29 (d, J=8.8 Hz, 1H, ArH), 8.08 (d, J=8.8 Hz, 1H, ArH), 7.87 (s, 1H, Pyrazole-H), 7.49 (s, 1H, Pyrazole-H), 6.00 (s, 1H, OH), 3.64 (d, J=8.2 Hz, 1H, CH), 4.50 (d, J=9.6 Hz, 1H, CH), 3.60-3.56 (m, 2H, CH₂), 2.59-2.55 (m, 2H, CH₂), 1.31 (s, 3H, CH₃). HRMS [C₁₉H₁₅F₃N₄O₃⁺]: calcd 407.1331, found 407.1267 [M+H]⁺; HRMS [C₁₉H₁₁F₃N₄NaO₃⁺]: calcd 429.1150, found 429.1099 [M+Nat. Purity:% (HPLC).

### (S)-1-(3-((4-Cyano-3-(trifluoromethyl)phenyl)amino)-2-hydroxy-2-methyl-3-oxopropyl)1H-pyrazole-4-carboxamide (16s)

Compound 16s was prepared in two steps. In the first step, (—(S)-tert-butyl (1-(3-((4-cyano-3-(trifluoromethyl)phenyl)amino)-2-hydroxy-2-methyl-3-oxopropyl)-1H-pyrazol-4-yl)carbamate 16u (an intermediate compound for 16s) was synthesized following General Procedure A per Scheme 1 as described in detail infra. The compound was purified by a silica gel column using hexanes and ethyl acetate (2:1) as eluent to afford the compound as white solid. Yield=69%. ¹H NMR (CDCl₃, 400 MHz) δ 9.13 (bs, 1H, NH), 8.18 (d, J=10.8 Hz, 1H), 8.02 (d, J=1.2 Hz, 1H), 7.94 (s, 1H), 7.89 (d, J=8.4 Hz, 1H), 7.77 (d, J=8.4 Hz, 1H), 7.66 (bs, C(O)NHC(O)), 5.79 (bs, 1H, OH), 4.70 (d, J=13.8 Hz, 1H), 4.32 (d, J=13.8 Hz, 1H), 1.52 (s, 3H), 1.50 (s, 9H). ¹⁹F NMR (CDCl₃, 400 MHz) δ −62.20. MS (ESI) m/z 480.23 [M−Hr; HRMS (ESI) m/z calcd for C₂₁H₂₂F₃N₅O₅ 382.1127 [(M-t-Boc)⁺H]+ found 382.1129 [[(M-t-Boc)+H]⁺.

The second step: To a solution of 16u (0.721 g, 2.05 mmol) in EtOH (10 mL) was added dropwise acetyl chloride (3 mL) at 0° C. and further stirred at rt for 3 h. After removing solvent under reduced pressure, the mixture was treated with ethyl acetate and hexane (2:1) to give the desired compound as a yellowish solid. Yield=95%. UV max 194.45, 270.45. ¹H NMR (400 MHz, DMSO-d₆) δ 10.39 (bs, 1H, NHC(O)), 8.46 (d, J=1.6 Hz, 1H), 8.20 (dd, J=8.6, 1.6 Hz, 1H), 8.08 (d, J=8.6 Hz, 1H), 8.05 (s, 1H), 7.75 (s, 1H), 7.55 (bs, 2H, C(O)NH₂), 6.99 (bs, 1H, OH), 4.45 (d, J=13.8 Hz, 1H), 4.28 (d, J=13.8 Hz, 1H), 1.34 (s, 3H). ¹⁹F NMR (DMSOd₆, decoupled) δ −61.13. MS (ESI) m/z 380.19 [M−H]⁻; HRMS (ESI) m/z calcd for C₁₆H₁₄F₃N₅O₃ 382.1127 [M+H]⁺, found 382.1282 [M+H]⁺_HPLC Purity: 98.75%.

### (S)-3-(4-Amino-1H-pyrazol-1-yl)-N-(4-cyano-3-(trifluoromethyl)phenyl)-2-hydroxy-2-methylpropanamide (16t)

To a solution of 16u (see below) (0.815 g, 0.0018 mol) in absolute EtOH (10 mL) was added acetyl chloride (0.4 mL, 5.4 mmol) at 0° C. and further stirred at rt for 3 h. After removing solvent under vacuum, the resulting mixture was purified by flash column chromatography using hexanes and ethyl acetate (1:1, v/v) to afford the designed compound as brown solid. Yield=91%. ¹H NMR (400 MHz, DMSO-d₆) δ 10.31 (bs, 1H, NH), 10.21 (bs, 2H, NH2), 8.20 (s, 1H), 7.98 (d, J=7.6 Hz, 1H), 7.77-7.73 (m, 2H), 7.62 (bs, 1H), 7.21 (bs, 1H), 6.28 (bs, 1H, OH), 4.23 (d, J=14.0 Hz, 1H), 4.04 (d, J=14.0 Hz, 1H), 1.04 (s, 3H); ¹⁹F NMR (acetone-d₆, decoupled) S 114.77. MS (ESI) m/z 354.08 [M+H]⁺; 351.98 [M−H]⁻.

### (S)-tert-Butyl (1-(3-((4-cyano-3-(trifluoromethyl)phenyl)amino)-2-hydroxy-2-methyl-3-oxopropyl)-1H-pyrazol-4-yl)carbamate (16u)

Compound 16u was prepared following General Procedure A per Scheme 1. The product was purified by a silica gel column using hexanes and ethyl acetate (2:1) as eluent to afford the designed compound as brown solid. 1H NMR (400 MHz, CDCl₃) δ 9.12 (bs, 1H, NH), 8.01 (d, J=1.6 Hz, 1H), 7.85 (dd, J=8.4, 1.6 Hz, 1H), 7.76 (d, J=8.4 Hz, 1H), 7.63 (bs, 1H), 7.43 (bs, 1H), 6.21 (bs, 1H, HN), 6.17 (bs, 1H, OH), 4.54 (d, J=14.0 Hz, 1H), 4.17 (d, J=14.0 Hz, 1H), 1.47 (s, 9H), 1.45 (s, 3H); ¹⁹F NMR (CDCl₃, decoupled) δ −62.21. MS (ESI) m/z 452.11 [M−H]⁻; 454.11 [M+H]⁺; 476.12 [M+Nat.

### (S)-3-(4-Acetamido-1H-pyrazol-1-yl)-N-(4-cyano-3-(trifluoromethyl)phenyl)-2-hydroxy-2-methylpropanamide (16v)

Under argon atmosphere, to a solution of 16t (0.17 g, 0.48 mmol) and triethylamine (0.16 mL, 1.15 mmol) in 10 mL of anhydrous DCM was added acetyl chloride (0.04 mL, 0.58 mmol) at ice-water bath. After stirring for 30 min, the temperature raised to rt and the mixture stirred for 2 h. The reaction mixture condensed on under reduced pressure, and then dispersed into 10 mL of ethyl acetate, washed with water, evaporated, dried over anhydrous MgSO₄, and evaporated to dryness. The mixture was purified with flash column chromatography using hexanes and ethyl acetate as eluent (2/1, v/v) to produce the designed compound as yellow solid. Yield=92%. 1H NMR (400 MHz, CDCl₃) δ 9.08 (bs, 1H, NH), 7.92 (bs, 1H, NH), 7.82-7.80 (m, 2H), 7.69 (d, J=8.4 Hz, 1H), 7.44 (bs, 1H), 7.15 (bs, 1H), 6.10 (bs, 1H, OH), 4.50 (d, J=14.0 Hz, 1H), 4.13 (d, J=14.0 Hz, 1H), 2.04 (s, 3H), 1.39 (s, 3H). ¹⁹F NMR (CDCl₃, decoupled) δ −62.20. MS (ESI) m/z 356.11 [M+H]⁺; 354.06 [M−H]⁻.

### (S)-3-(4-(2-Chloroacetamido)-1H-pyrazol-1-yl)-N-(4-cyano-3-(trifluoromethyl)phenyl)-2-hydroxy-2-methylpropanamide (16w)

Under argon atmosphere, to a solution of 16t (0.17 g, 0.48 mmol) and triethylamine (0.16 mL, 1.15 mmol) in 10 mL of anhydrous DCM was added 2-chloroacetyl chloride (0.04 mL, 0.58 mmol) in an ice-water bath. After stirring for 30 min, the temperature raised to rt and the mixture stirred for 2 h. The reaction mixture condensed under vacuum, and then dispersed into 10 mL of ethyl acetate, washed with water, evaporated, dried over anhydrous MgSO₄, and evaporated to dryness. The mixture was purified with flash column chromatography using hexanes and ethyl acetate as eluent (2/1, v/v) to produce the titled compound as yellow solids. Yield=68%. 1H NMR (400 MHz, CDCl₃) δ 9.12 (bs, 1H, NH), 8.12 (bs, 1H, NH), 7.99 (d, J=1.6 Hz, 1H), 7.92 (bs, 1H), 7.88 (dd, J=8.6, 1.6 Hz, 1H), 7.78 (d, J=8.6 Hz, 1H), 7.61 (bs, 1H), 6.11 (bs, 1H, OH), 4.60 (d, J=13.8 Hz, 1H), 4.23 (d, J=13.8 Hz, 1H), 4.17 (s, 2H), 1.47 (s, 3H); 19F NMR (CDCl₃, decoupled) δ −62.19. MS (ESI) m/z 452.01 [M+Na]⁺; 428.00 [M−H]⁻.

### (S)-Methyl(1-(3-((4-cyano-3-(trifluoromethyl)phenyl)amino)-2-hydroxy-2-methyl-3-oxopropyl)-1H-pyrazol-4-yl)carbamate (16x)

Under argon atmosphere, to a solution of 16t (0.17 g, 0.48 mmol) and triethylamine (0.16 mL, 1.15 mmol) in 10 mL of anhydrous DCM was added methyl carbonochloridate (0.04 mL, 0.58 mmol) in an ice-water bath. After stirring for 30 min, the temperature raised to rt and the mixture stirred for 2 h. The reaction mixture condensed on under vacuum, and then dispersed into 10 mL of ethyl acetate, washed with water, evaporated, dried over anhydrous MgSO₄, and evaporated to dryness. The mixture was purified with flash column chromatography using hexanes and ethyl acetate as eluent (2/1, v/v) to produce the titled compound as white solid. Yield=71%. ¹H NMR (400 MHz, CDCl₃) δ 9.07 (bs, 1H, C(O)NH), 7.91 (s, 1H, ArH), 7.79 (d, J=7.2 Hz, 1H, ArH), 7.69 (d, J=7.2 Hz, 1H, ArH), 7.57 (s, 1H, ArH), 7.40 (s, 1H, ArH), 6.33 (bs, 1H, NH), 6.08 (bs, 1H, OH), 4.50 (d, J=13.6 Hz, 1H, CH₂), 4.12 (d, J=13.6 Hz, 1H, CH₂), 3.67 (s, 3H, NH(CO)OCH₃), 1.39 (s, 3H, CH₃); 19F NMR (CDCl₃, decoupled) δ −62.21. MS (ESI) m/z 410.30 [M−H]⁻; 413.21 [M+H]⁺

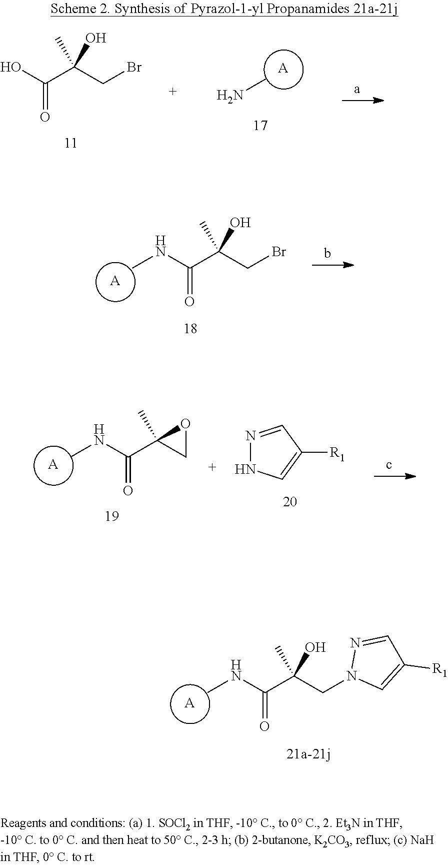

Compound 21a was prepared following General Procedure A per Scheme 2 where 17 was 5-amino-3-(trifluoromethyl)picolinonitrile. The product was purified by a silica gel column using hexanes and ethyl acetate (1:1) as eluent to afford 0.50 g of the titled compound as white solid. Yield=60.2%. ¹H NMR (400 MHz, DMSO-d₆) δ 10.64 (s, 1H, NH), 9.32 (d, J=2.0 Hz, 1H, ArH), 8.82 (d, J=2.0 Hz, 1H, ArH), 7.75 (d, J=4.8 Hz, 1H, Pyrazole-H), 7.40 (d, J=4.0 Hz, 1H, Pyrazole-H), 6.41 (s, 1H, OH), 4.39 (d, J=14.0 Hz, 1H, CH), 4.22 (d, J=14.0 Hz, 1H, CH), 1.36 (s, 3H, CH₃). HRMS [C14H12F₄N₅O₂⁺]: calcd 358.0927, found 358.0932.

### (S)—N-(6-Cyano-5-(trifluoromethyl)pyridin-3-yl)2-hydroxy-2-methyl-3-(4-(trifluoromethyl) 1H-pyrazol-1-yl)propanamide (21b)

Compound 21b was prepared following General Procedure A per Scheme 2 where 17 was 5-amino-3-(trifluoromethyl)picolinonitrile. The product was purified by a silica gel column using DCM and ethyl acetate (19:1) as eluent to afford 0.18 g of the titled compound as white solid. Yield=60%. ¹H NMR (400 MHz, DMSO-d₆) δ 10.63 (s, 1H, NH), 9.31 (s, 1H, ArH), 8.80 (s, 1H, ArH), 8.32 (s, 1H, Pyrazole-H), 7.81 (s, 1H, Pyrazole-H), 6.48 (s, 1H, OH), 4.55 (d, J=14.0 Hz, 1H, CH), 4.37 (d, J=14.0 Hz, 1H, CH), 1.42 (s, 3H, CH₃). HRMS [C₁₅H₁₂F₆N₅O₂⁺]: calcd 408.0892, found 408.0890[M+H]⁺. Purity: 96.81% (HPLC).

### (S)-3-(4-Cyano-1H-pyrazol-1-yl)-N-(6-cyano-5-(trifluoromethyl)pyridin-3-yl)-2-hydroxy-2-methylpropanamide (21c)

Compound 21c was prepared following General Procedure A per Scheme 2 where 17 was 5-amino-3-(trifluoromethyl)picolinonitrile. The product was purified by a silica gel column using hexanes and ethyl acetate (2:1) as eluent to the titled compound as off-white solid. Yield=52%. MP 169.7-169.9° C.; UV max 195.45, 274.45; ¹H NMR (400 MHz, CDCl₃) δ 9.17 (bs, 1H, NH), 8.83 (s, 1H), 8.67 (d, J=1.6 Hz, 1H), 7.92 (s, 1H), 7.85 (s, 1H), 5.58 (s, OH), 4.73 (d, J=14.0 Hz, 1H), 4.34 (d, J=14.0 Hz, 1H), 1.53 (s, 3H); 19F NMR (CDCl₃, decoupled) δ −62.11. MS (ESI) m/z 363.1 [M−H]⁻; 365.0 [M+H]⁺; HRMS (ESI) m/z calcd for C₁₅H₁₁F₃N₆O₂ 365.0974 [M+H]⁺ found 365.0931 [M+H]⁺; 387.0754 [M+Na]⁺.

### (S)-tert-Butyl (1-(3-((6-cyano-5-(trifluoromethyl)pyridin-3-yl)amino)-2-hydroxy-2-methyl-3-oxopropyl)-1H-pyrazol-4-yl)carbamate (21d)

Compound 21d was prepared following General Procedure A per Scheme 2 where 17 was 5-amino-3-(trifluoromethyl)picolinonitrile. The product was purified by a silica gel column using hexanes and ethyl acetate (2:1) as eluent to afford the designed compound brown solid. Yield=60%. ¹H NMR (400 MHz, CDCl₃) δ 9.28 (bs, 1H, NH), 8.80 (s, 1H), 7.63 (bs, 1H), 7.43 (bs, 1H), 6.29 (bs, 1H, NH), 6.21 (bs, 1H, OH), 4.55 (d, J=14.0 Hz, 1H), 4.17 (d, J=14.0 Hz, 1H), 1.47 (s, 3H); 19F NMR (CDCl₃, decoupled) δ −62.11. MS (ESI) m/z 453.16 [M−H]⁻; 477.16 [M+Na]⁺.

### (S)—N-(3-Chloro-4-cyanophenyl)-3-(4-fluoro-1H-pyrazol-1-yl)-2-hydroxy-2-methylpropanamide (21e)

Compound 21e was prepared following General Procedure A per Scheme 2 where 17 was 4-amino-2-chlorobenzonitrile. The product was purified by a silica gel column using hexanes and ethyl acetate (2:1) as eluent to afford the designed compound white solid. Yield=71%. 1H NMR (400 MHz, CDCl₃) δ 8.97 (bs, 1H, NH), 7.87 (d, J=2.0 Hz, 1H), 7.60 (d, J=8.4 Hz, 1H), 7.45 (dd, J=8.4, 2.0 Hz, 1H), 7.36 (dd, J=8.8, J=4.0 Hz, 1H), 5.86 (bs, 1H, OH), 4.55 (d, J=14.0 Hz, 1H), 4.16 (d, J=14.0 Hz, 1H), 1.46 (s, 3H); 19F NMR (CDCl₃, decoupled) δ −176.47. HRMS (ESI) m/z calcd for C₁₄H₁₂FN₄O₂ 323.0711 [M+H]⁺ found 323.0710 [M+H]⁺.

### (S)-3-(4-Fluoro-1H-pyrazol-1-yl)-2-hydroxy-2-methyl-N-(4-nitro-3-(trifluoromethyl)phenyl)propanamide (21f)

Compound 21f was prepared following General Procedure A per Scheme 2 where 17 was 4-nitro-3-(trifluoromethyl)aniline. The product was purified by a silica gel column using hexanes and ethyl acetate (2:1) as eluent to afford the designed compound yellowish solid. Yield=67%. ¹H NMR (400 MHz, CDCl₃) δ 9.14 (bs, 1H, NH), 8.01 (s, 1H), 7.97-7.91 (m, 2H), 7.38 (d, J=3.6 Hz, 1H), 7.35 (d, J=4.4 Hz, 1H), 5.95 (s, 1H, OH), 4.56 (d, J=14.0 Hz, 1H), 4.17 (d, J=14.0 Hz, 1H), 1.48 (s, 3H); 19F NMR (CDCl₃, decoupled) δ −60.13, −176.47. MS (ESI) m/z 375.08 [M−H]⁻; 377.22 [M+H]⁺; 399.04 [M+Na]⁺.

### (S)-5-(3-(4-Fluoro-1H-pyrazol-1-yl)-2-hydroxy-2-methylpropanamido)picolinamide (21g)

Compound 21g was prepared following General Procedure A per Scheme 2 where 17 of step a was 5-cyano-6-(trifluoromethyl)picolinamide. In step c, to a solution of 4-fluoropyrazole (20; 0.20 g, 0.0023237 mol) in anhydrous THF (5 mL) which was cooled in an ice water bath under an argon atmosphere, was added sodium hydride (60% dispersion in oil, 0.28 g, 0.0069711 mol). After addition, the resulting mixture was stirred for three hours. (R)-3-Bromo-N-(6-cyanopyridin-3-yl)-2-hydroxy-2-methylpropanamide 18 (0.66 μg, 0.0023237 mol) was added to above solution, and the resulting reaction mixture was allowed to stir overnight at rt under argon. The reaction was quenched by water and extracted with ethyl acetate. The organic layer was washed with brine, dried with MgSO₄, filtered, and concentrated under vacuum. The product was purified by a silica gel column using DCM and methanol (9:1) as eluent to afford 0.10 g of the titled compound as white solid. Yield=14.1%. ¹H NMR (400 MHz, DMSO-d₆) δ 10.08 (s, 1H, NH), 8.89 (d, J=2.4 Hz, 1H, ArH), 8.30 (dd, J=8.2 Hz, J=2.4 Hz, 1H, ArH), 8.01 (s, 1H, NH), 7.98 (d, J=8.2 Hz, 1H, ArH), 7.73 (d, J=4.4 Hz, 1H, Pyrazole-H), 7.51 (s, 1H, NH), 7.42 (d, J=4.0 Hz, 1H, Pyrazole-H), 6.24 (s, 1H, OH), 4.38 (d, J=14.0 Hz, 1H, CH), 4.42 (d, J=14.0 Hz, 1H, CH), 1.34 (s, 3H, CH₃). HRMS [C₁₃H₁₅FN₅O₃⁺]: calcd 308.1159, found 308.1177 [M+H]⁺; HRMS [C₁₃H₁₄FN₅NaO₃⁺] calcd 330.0978, found 330.0987 [M+Na]⁺.

### (S)-3-(4-Fluoro-1H-pyrazol-1-yl)-2-hydroxy-2-methyl-N-(quinazolin-6-yl)propanamide (21h)

Compound 21h was prepared following General Procedure A per Scheme 2 where 17 of step a was quinazolin-6-amine. In step c, too a solution of 4-fluoro-pyrazole (20; 0.20 g, 0.0023237 mol) in anhydrous THF (5 mL), which was cooled in an ice water bath under an argon atmosphere, was added sodium hydride (60% dispersion in oil, 0.28 g, 0.0069711 mol). After addition, the resulting mixture was stirred for three hours. (R)-3-Bromo-2-hydroxy-2-methyl-N-(quinazolin-6-yl) propanamide (18; 0.72 g, 0.0023237 mol) was added to above solution, and the resulting reaction mixture was allowed to stir overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (19:1) as eluent to afford 50 mg of the titled compound as yellow solid. Yield=13.7%. ¹H NMR (400 MHz, DMSO-d₆) δ 10.10 (s, 1H, NH), 9.54 (s, 1H, ArH), 9.21 (s, 1H, ArH), 8.64 (d, J=2.4 Hz, 1H, ArH), 8.22 (dd, J=8.6 Hz, J=2.4 Hz, 1H, ArH), 7.97 (d, J=8.6 Hz, 1H, ArH), 7.75 (d, J=4.8 Hz, 1H, Pyrazole-H), 7.43 (d, J=4.0 Hz, 1H, Pyrazole-H), 6.26 (s, 1H, OH), 4.42 (d, J=14.0 Hz, 1H, CH), 4.25 (d, J=14.0 Hz, 1H, CH), 1.36 (s, 3H, CH₃). Mass (ESI, Negative): 314.05[M−H]⁻.

### (S)—N-(2-Chloropyridin-4-yl)-3-(4-fluoro-1H-pyrazol-1-yl)-2-hydroxy-2-methylpropanamide

Compound 21i was prepared in two steps. In the first step, (R)-3-bromo-N-(2-chloropyridin-4-yl)-2-hydroxy-2-methylpropanamide as intermediate compound was synthesized following Scheme 2 where 17 was 2-chloropyridin-4-amine. Thionyl chloride (11.2 mL, 0.154 mol) was added dropwise to a cooled solution (less than 4° C.) of 11 (18.3 g, 0.100 mol) in 100 mL of THF under an argon atmosphere. The resulting mixture was stirred for 3 h under the same condition. To this was added Et₃N (25.7 mL, 0.185 mol) and stirred for 20 min under the same condition. After 20 min, 2-chloropyridin-4-amine (17; 9.89 g, 0.077 mol), 100 mL of THF were added and then the mixture was allowed to stir overnight at room temperature. The solvent was removed under reduced pressure to give a solid which was treated with 100 mL of H₂O, extracted with ethyl acetate (2×50 mL). The combined organic extracts were washed with saturated NaHCO₃ solution (2×100 mL) and brine (100 mL). The organic layer was dried over MgSO₄ and concentrated under reduced pressure to give a solid which was purified from column chromatography using ethyl acetate and DCM (80:20) to give a solid. This solid was recrystallized from DCM and hexane to give 12.6 g of intermediate compound as a light-yellow solid. Yield=43%. ¹H NMR (400 MHz, CDCl₃) δ 9.06 (bs, 1H, NH), 8.31 (d, J=5.6 Hz, 1H), 7.77 (d, J=0.8 Hz, 1H), 7.45 (dd, J=5.6, 0.8 Hz, 1H), 4.81 (bs, 1H, OH), 3.97 (d, J=10.6 Hz, 1H), 3.60 (d, J=10.6 Hz, 1H), 1.64 (s, 3H). MS (ESI) m/z 295.28 [M+H]⁺.

The second step: 21i was prepared following General Procedure A per Scheme 2 where 18 was (R)-3-bromo-N-(2-chloropyridin-4-yl)-2-hydroxy-2-methylpropanamide. The product was purified by a silica gel column using hexanes and ethyl acetate (2:1) as eluent to give the desired compound as white solid. Yield=55%. ¹H NMR (400 MHz, CDCl₃) δ 8.90 (bs, 1H, NH), 8.26 (d, J=5.6 Hz, 1H), 7.63 (s, 1H), 7.75 (d, J=4.2 Hz, 1H), 7.33 (d, J=4.2 Hz, 1H), 7.31 (dd, J=5.6, 1.2 Hz, 1H), 5.88 (s, 1H, OH), 4.53 (d, J=13.6 Hz, 1H), 4.14 (d, J=13.6 Hz, 1H), 1.45 (s, 3H); ¹⁹F NMR (CDCl₃, decoupled) δ −176.47. MS (ESI) m/z 298.98 [M+H]⁺; 296.96 [MH]⁻.

### (S)—N-(4-Cyano-2-iodo-5-(trifluoromethyl)phenyl)-3-(4-fluoro-1H-pyrazol-1-yl)-2-hydroxy-2-methylpropanamide (21j)

Compound 21j was prepared following General Procedure A per Scheme 2 where 17 of step a was 4-cyano-2-iodoaniline. In step c, 20 was 4-fluoro-1H-pyrazole (0.09 g, 0.001048 mol). The product was purified by a silica gel column using hexanes and ethyl acetate (2:1 to 1:1) as eluent to afford 0.32 g of the titled compound as white solid. Yield=64%. ¹H NMR (400 MHz, CDCl₃) δ 9.60 (s, 1H, NH), 8.76 (s, 1H, ArH), 8.69 (s, 1H, ArH), 7.76 (d, J=4.8 Hz, 1H, Pyrazole-H), 7.36 (d, J=4.4 Hz, 1H, Pyrazole-H), 6.85 (s, 1H, OH), 4.39 (d, J=14.0 Hz, 1H, CH), 4.20 (d, J=14.0 Hz, 1H, CH), 1.41 (s, 3H, CH₃). Mass (ESI, Negative): 481.00 [M−H]⁻.

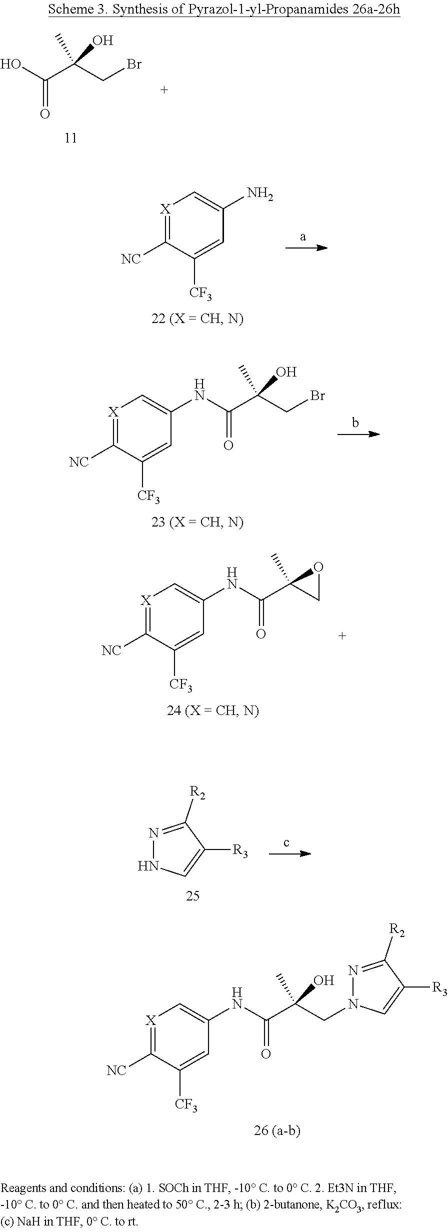

### (S)-3-(4-Bromo-3-fluoro-1H-pyrazol-1-yl)-N-(4-cyano-3-(trifluoromethyl)phenyl)-2-hydroxy-2-methylpropanamide (26a)

Compound 26a was prepared following General Procedure A per Scheme 3. To a solution of 4-bromo-3-fluoro-pyrazole (25; 0.30 g, 0.001819 mol) in anhydrous THF (10 mL), which was cooled in an ice water bath under an argon atmosphere, was added sodium hydride (60% dispersion in oil, 0.26 g, 0.006365 mol). After addition, the resulting mixture was stirred for three hours. 23 (where X is CH; 0.64 g, 0.001819 mol) was added to above solution, and the resulting reaction mixture was allowed to stir overnight at rt under argon. The reaction was quenched with water and extracted with ethyl acetate. The organic layer was washed with brine, dried with MgSO₄, filtered, and concentrated under vacuum. The product was purified by a silica gel column using ethyl acetate and hexanes (2:1) as eluent to afford 0.34 g of the titled compound as pinkish solid. Yield=34%. ¹H NMR (400 MHz, DMSO-d₆) δ 10.38 (s, 1H, NH), 8.45 (d, J=2.0-1.6 Hz, 1H, ArH), 8.23 (dd, J=8.2 Hz, J=2.0 Hz, 1H, ArH), 8.11 (d, J=8.2 Hz, 1H, ArH), 7.82 (d, J=2.0 Hz, 1H, Pyrazole-H), 6.35 (s, 1H, OH), 4.35 (d, J=14.0 Hz, 1H, CH), 4.04 (d, J=14.0 Hz, 1H, CH), 1.37 (s, 3H, CH₃). m.p 110-112° C. HRMS [C₁₅H₁₂BrF₄N₄O₂⁺]: calcd 435.0080, found 435.0080[M+H]⁺. Purity: 96.98% (HPLC).

### (S)—N-(4-Cyano-3-(trifluoromethyl)phenyl)-3-(3-fluoro-4-(4-fluorophenyl)-1H-pyrazol-lyl)-2-hydroxy-2-methylpropanamide (26b)

Compound 26b was prepared by Suzuki reaction mixing 26a (0.20 g, 0.4596 mmol), 4-fluoro boronic acid (77 mg, 0.5515 mmol), Pd(II)(OAc)₂ (2-3 mg, 0.009192 mmol), PPh₃ (7-8 mg, 0.02758 mmol), and K₂CO₃ (0.13 g, 0.965 mmol) into ACN (4-5 mL) and H₂O (2-3 mL). The mixture was degassed and refilled with argon three times. The resulting reacting mixture was heated at reflux for 3 h under argon. The product was purified by a silica gel column using hexanes and ethyl acetate (2:1 to 1:1) as eluent to afford 51 mg of the titled compound as yellowish solid. Yield=25%. ¹H NMR (400 MHz, CDCl₆) δ 9.12 (s, 1H, NH), 8.06 (d, J=1.6 Hz, 1H, ArH), 7.85 (dd, J=8.2 Hz, J=1.6 Hz, 1H, ArH), 7.77 (d, J=8.2 Hz, 1H, ArH), 7.51 (d, J=3.0 Hz, 1H, Pyrazole-H), 7.43-7.40 (m, 2H, ArH), 7.08-7.04 (m, 2H, ArH), 4.57 (d, J=10.5 Hz, 1H, CH), 4.17 (d, J=10.5 Hz, 1H, CH), 1.26 (s, 3H, CH₃). HRMS [C₂₁H₁₆F₅N₄O₂⁺]: calcd 451.1193, found 451.1196[M+H]⁺. Purity:% (HPLC).

### (S)-3-(3-Bromo-4-cyano-1H-pyrazol-1-yl)-N-(4-cyano-3-(trifluoromethyl)phenyl)-2-hydroxy-2-methylpropanamide (26c)

Compound 26c was prepared following General Procedure A per Scheme 3 where X of 22 (step a) is CH and 25 of step c is 3-bromo-4-cyano-pyrazole. The product was purified by a silica gel column using ethyl acetate and hexanes (2:1) as eluent to afford 0.10 g of the titled compound as off-white solid. Yield=20%. ¹H NMR (400 MHz, DMSO-d₆) δ 10.32 (s, 1H, NH), 8.50 (s, 1H, Pyrazole-H), 8.41 (s, 1H, ArH), 8.20 (d, J=8.4 Hz, 1H, ArH), 8.11 (d, J=8.4 Hz, 1H, ArH), 6.47 (s, 1H, OH), 4.52 (d, J=13.6 Hz, 1H, CH), 4.33 (d, J=13.6 Hz, 1H, CH), 1.41 (s, 3H, CH₃). HRMS [C₁₆H₁₂BrF₃N₅O₂⁺]: calcd 442.0126, found 442.0109[M+H]⁺. Purity: 98.84% (HPLC).

### (S)-3-(3-Chloro-4-methyl-1H-pyrazol-1-yl)-N-(4-cyano-3-(trifluoromethyl)phenyl)-2-hydroxy-2-methylpropanamide (26d)

Compound 26d was prepared following General Procedure A per Scheme 3 where X of 22 (step a) is CH and 25 of step c is 3-chloro-4-methyl-pyrazole. The product was purified by a silica gel column using DCM and ethyl acetate (98:2 to 95:5) as eluent to afford 0.27 g of the titled compound as white solid. Yield=54%. ¹H NMR (400 MHz, DMSO-d₆) δ 10.33 (s, 1H, NH), 8.42 (d, J=0.8 Hz, 1H, ArH), 8.21 (dd, J=8.4 Hz, J=0.8 Hz, 1H, ArH), 8.10 (d, J=8.2 Hz, 1H, ArH), 7.50 (s, 1H, Pyrazole-H), 6.29 (s, 1H, OH), 4.36 (d, J=14.4 Hz, 1H, CH), 4.18 (d, J=14.4 Hz, 1H, CH), 1.91 (s, 3H, CH₃), 1.35 (s, 3H, CH₃). HRMS [C₁₆H₁₅ClF₃N₄O₂⁺]: calcd 387.0836, found 387.0839[M+H]⁺. Purity: 97.07% (HPLC).

### (S)-3-(3-Bromo-4-chloro-1H-pyrazol-1-yl)-N-(4-cyano-3-(trifluoromethyl)phenyl)-2-hydroxy-2-methylpropanamide (26e)

Compound 26e was prepared following General Procedure A per Scheme 3 where X of 22 (step a) is CH and 25 of step c is 3-bromo-4-chloro-pyrazole. The product was purified by a silica gel column using DCM and ethyl acetate (95:5) as eluent to afford 0.25 g of the titled compound as white solid. Yield=50%. ¹H NMR (400 MHz, DMSO-d₆) δ 10.34 (s, 1H, NH), 8.41 (s, 1H, ArH), 8.20 (d, J=8.8 Hz, 1H, ArH), 8.11 (d, J=8.8 Hz, 1H, ArH), 7.93 (s, 1H, Pyrazole-H), 6.39 (s, 1H, OH), 4.43 (d, J=14.0 Hz, 1H, CH), 4.25 (d, J=14.0 Hz, 1H, CH), 1.38 (s, 3H, CH₃). HRMS [C₁₅H₁₂BrClF₃N₄O₂⁺]: calcd 450.9784, found 450.9807 [M+H]⁺. Purity: 96.55% (HPLC).

### (S)-3-(4-Bromo-3-fluoro-1H-pyrazol-1-yl)-N-(6-cyano-5-(trifluoromethyl)pyridin-3-yl)-2-hydroxy-2-methylpropanamide (26f)

Compound 26f was prepared following General Procedure A per Scheme 3 where X of 22 (step a) is N and 25 of step c is 4-bromo-3-fluoro-pyrazole The product was purified by a silica gel column using hexanes and ethyl acetate (2:1 to 1:1) as eluent to afford 0.28 g of the titled compound as white solid. Yield=54%. ¹H NMR (400 MHz, DMSO-d₆) δ 10.67 (s, 1H, NH), 9.32 (d, J=2.0 Hz, 1H, ArH), 8.82 (d, J=2.0 Hz, 1H, ArH), 7.85 (d, J=2.0 Hz 1H, Pyrazole-H), 6.47 (s, 1H, OH), 4.35 (d, J=14.0 Hz, 1H, CH), 4.17 (d, J=14.0 Hz, 1H, CH), 1.39 (s, 3H, CH₃). HRMS [C₁₅H₁₂BrClF₃N₄O₂⁺]: calcd 434.9954, found 435.9997 [M+H]⁺. Purity: 93.41% (HPLC).

### (S)-3-(3-Bromo-4-cyano-1H-pyrazol-1-yl)-N-(6-cyano-5-(trifluoromethyl)pyridin-3-yl)-2-hydroxy-2-methylpropanamide (26g)

Compound 26g was prepared following General Procedure A per Scheme 3 where X of 22 (step a) is N and 25 of step c is 3-bromo-4-cyano-pyrazole. The product was purified by a silica gel column using hexanes and ethyl acetate (2:1) as eluent to afford the titled compound as white solid. Yield=81%. MP 172.5-173.6° C.; ¹H NMR (400 MHz, DMSO-d₆) δ 10.60 (bs, 1H, NH), 9.29 (s, 1H), 8.79 (s, 1H), 8.53 (s, 1H), 6.59 (s, OH), 4.50 (d, J=14.0 Hz, 1H), 4.32 (d, J=14.0 Hz, 1H), 1.43 (s, 3H); 19F NMR (CDCl₃, decoupled) δ −61.25. MS (ESI) m/z 442.1 [M−Hr; HRMS (ESI) m/z calcd for C₁₅H₁₀BrF₃N₆O₂ 443.0079 [M+H]+ found 443.0083 [M+H]⁺; 464.9903 [M+Na]⁺.

### (S)-3-(4-Cyano-3-phenyl-1H-pyrazol-1-yl)-N-(6-cyano-5-(trifluoromethyl)pyridin-3-yl)-2-hydroxy-2-methylpropanamide (26h)

A flask equipped with a reflux condenser, a septum inlet and a magnetic stirring bar was charged with 26g (0.053 g, 0.23 mmol), tetrakis(triphenylphosphine) palladium (0) (9 mg, 0.07 mmol), and phenyl boronic acid (35 mg, 0.28 mmol) in THF/MeOH (5 mL/1 mL) with sodium carbonate (50 mg, 0.48 mmol) in deoxygenated water (1 mL) was stirred and heated to reflux for 2 h until starting material was not detectable on TLC. The mixture was cooled to rt and the solvent was removed in vacuo and then poured into ethyl acetate (10 mL) and extracted with ethyl acetate. The combined organic layers were washed with sat. NH₄Cl, water and dried over MgSO₄. The solvent was removed in vacuo and then purified by flash column chromatography on silica gel using hexanes and ethyl acetate (1:1) as eluent to give 36 mg of the targeted compound as yellowish solid. Yield=69%. MP 112.3-124.4° C.; ¹H NMR (400 MHz, CDCl₃) δ 9.17 (bs, 1H, NH), 8.76 (s, 1H), 8.60 (s, 1H), 7.77 (s, 1H), 7.57-7.52 (m, 3H), 7.18 (d, J=8.8 Hz, 2H), 5.32 (s, OH), 4.60 (d, J=14.0 Hz, 1H), 4.23 (d, J=14.0 Hz, 1H), 1.47 (s, 3H). ¹⁹F NMR (CDCL₃, decoupled) δ −62.09. MS (ESI) m/z 439.2 [M−H]⁻; HRMS (ESI) m/z calcd for C₂₁H₁₅F₃N₆O₂ 441.1287 [M+H]⁺ found 441.1291 [M+H]⁺; 463.1111 [M+Na]⁺.

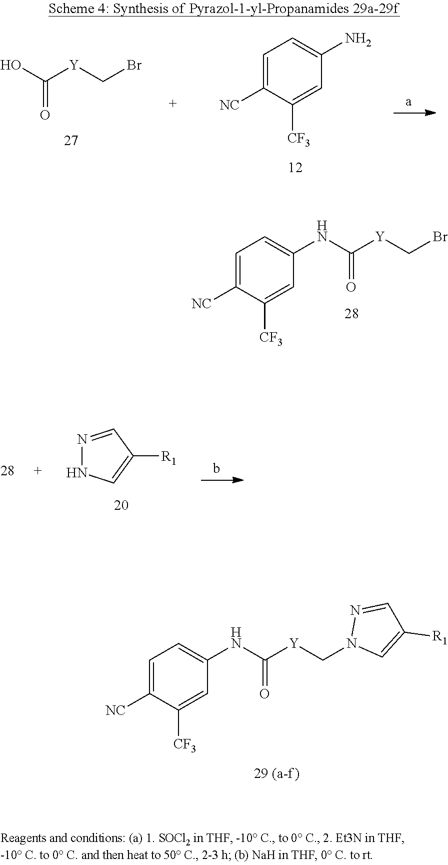

### (R)—N-(4-Cyano-3-(trifluoromethyl)phenyl)-3-(4-fluoro-1H-pyrazol-1-yl)-2-hydroxy-2-methylpropanamide (29a)

Compound 29a was prepared following General Procedure A per Scheme 1 as exemplified above for 10, except that instead of 11, the opposite isomer was used [(S)-11 or (S)-3-bromo-2-hydroxy-2-methylpropanoic acid]. The product was purified by a silica gel column using hexanes and ethyl acetate (1:1) as eluent to afford the titled compound as yellowish solid. Yield=64%. [α]_{D}²⁴+126.7° (c=1.0, MeOH); ¹H NMR (400 MHz, CDCl₃) δ 9.07 (bs, 1H, NH), 8.01 (d, J=2.0 Hz, 1H), 7.95 (dd, J=8.4, J=2.0 Hz, 1H), 7.78 (d, J=8.4 Hz, 1H), 7.38 (d, J=4.0 Hz, 1H), 7.34 (d, J=4.4 Hz, 1H), 5.92 (s, OH), 4.54 (d, J=14.0 Hz, 1H), 4.16 (d, J=14.4 Hz, 1H), 1.47 (s, 3H); ¹⁹F NMR (CDCl₃, decoupled) δ −62.23, −176.47. HRMS (ESI) m/z calcd for C₁₅H₁₂F₄N₄O₂: 357.0975 [M+H]⁺. Found: 357.0984 [M+H]⁺.

### N-(4-Cyano-3-(trifluoromethyl)phenyl)-3-(4-fluoro-1H-pyrazol-1-yl)-2-methylpropanamide (29b)

Compound 29b was prepared following General Procedure A per Scheme 4 where Y in 27 is tertiary carbon [CH(CH₃)] instead of quaternary [C(OH)(CH₃)] like all previous compounds. To a solution of 4-fluoro-pyrazole (20; 0.20 g, 0.0023237 mol) in anhydrous THF (5 mL), which was cooled in an ice water bath under an argon atmosphere, was added sodium hydride (60% dispersion in oil, 0.28 g, 0.0069711 mol). After addition, the resulting mixture was stirred for three hours. 3-Bromo-N-(4-cyano-3-(trifluoromethyl)phenyl)-2-methylpropanamide (28; 0.78 g, 0.0023237 mol) was added to above solution, and the resulting reaction mixture was allowed to stir overnight at rt under argon. The reaction was quenched with water and extracted with ethyl acetate. The organic layer was washed with brine, dried with MgSO₄, filtered, and concentrated under vacuum. The product was purified by a silica gel column using hexanes and ethyl acetate (1:1) as eluent to afford 0.050 g of the titled compound as yellowish solid. Yield=6.3%. ¹H NMR (400 MHz, DMSO-d₆) δ 10.77 (s, 1H, NH), 8.25 (s, 1H, ArH), 8.10 (d, J=8.2 Hz, 1H, ArH), 7.96 (d, J=8.2 Hz, 1H, ArH), 7.85 (d, J=4.4 Hz, 1H, Pyrazole-H), 7.47 (d, J=4.4 Hz, 1H, Pyrazole-H), 4.35-4.30 (m, 1H, CH), 4.12-4.07 (m, 1H, CH), 3.12-3.10 (m, 1H, CH), 1.22 (d, J=6.8 Hz, 3H, CH₃). Mass (ESI, Positive): 341.14 [M+H]⁺.

### N-(4-Cyano-3-(trifluoromethyl)phenyl)-3-(4-fluoro-1H-pyrazol-1-yl)propanamide (29c)

Compound 29c was prepared following General Procedure A per Scheme 4 where Y in 27 is secondary carbon [CH₂]] instead of quarternary [C(OH)(CH₃)]. To a solution of 4-fluoropyrazole (20; 0.20 g, 0.0023237 mol) in anhydrous THF (5 mL), which was cooled in an ice water bath under an argon atmosphere, was added sodium hydride (60% dispersion in oil, 0.28 g, 0.0069711 mol). After addition, the resulting mixture was stirred for three hours. 3-Bromo-N-(4-cyano-3-(trifluoromethyl)phenyl)propanamide (28; 0.75 g, 0.0023237 mol) was added to above solution, and the resulting reaction mixture was allowed to stir overnight at rt under argon. The reaction was quenched with water and extracted with ethyl acetate. The organic layer was washed with brine, dried with MgSO₄, filtered, and concentrated under vacuum. The product was purified by a silica gel column using DCM and methanol (19:1) as eluent to afford 0.75 mg of the titled compound as white solid. Yield=10%. ¹H NMR (400 MHz, DMSO-d₆) δ 10.81 (s, 1H, NH), 8.25 (d, J=2.4 Hz, 1H, ArH), 8.10 (dd, J=8.8 Hz, J=2.4 Hz, 1H, ArH), 7.95 (d, J=8.8 Hz, 1H, ArH), 7.88 (s, 1H, Pyrazole-H), 7.46 (s, 1H, Pyrazole-H), 4.35 (t, J=6.0 Hz, 2H, CH2), 2.79 (t, J=6.0 Hz, 2H, CH2). Mass (ESI, Negative): 325.03 [M−H; ; (ESI, Positive): [M+H]⁺.

### (S)-4-(5-((4-Fluoro-1H-pyrazol-1-yl)methyl)-5-methyl-2,4-dioxooxazolidin-3-yl)-2-(trifluoromethyl) benzonitrile (29d)

Preparation of 29d proceeded by cyclization of the 2-methyl-2-hydroxy-propanamide linker of 10 form an oxazolidinedione ring system. To a solution of 10 (0.234 g, 0.0006568 mol) in anhydrous pyridine (8 mL) was added 1,1′-carbonyldiimidazole (CDI) (0.16 g, 0.0009825 mol). After addition, the resulting mixture was allowed to stir overnight at rt under argon. The reaction was quenched with water and extracted with ethyl acetate. The organic layer was washed with brine, dried with MgSO₄, filtered, and concentrated under vacuum. The product was purified by a silica gel column using hexanes and ethyl acetate (2:1) as eluent to afford 0.134 g of the titled compound as white foam. Yield=42%. ¹H NMR (400 MHz, DMSO-d₆) δ 8.41 (d, J=8.0 Hz, 1H, ArH), 7.98 (s, 1H, ArH), 7.94 (d, J=4.0 Hz, 1H, Pyrazole-H), 7.85 (d, J=8.2 Hz, 1H, ArH), 7.58 (d, J=4.4 Hz, 1H, Pyrazole-H), 4.78 (d, J=14.8 Hz, 1H, CH), 4.69 (d, J=14.8 Hz, 1H, CH), 1.71 (s, 3H, CH₃). HRMS [C₁₆H₁₁F₄N₄Q₃⁺]: calcd 383.0767, found 383.0726 [M+H]⁺. Purity: 97.64% (HPLC).

### (S)-3-(4-Amino-1H-pyrazol-1-yl)-1-((4-cyano-3-(trifluoromethyl)phenyl)amino)-2-methyl-1-oxopropan-2-yl 2-chloroacetate (29e)

Under argon atmosphere, to a solution of 16t (0.17 g, 0.48 mmol) and triethylamine (0.16 mL, 1.15 mmol) in 10 mL of anhydrous DCM was added 2-chloroacetyl chloride (0.04 mL, 0.58 mmol) in an ice-water bath. After stirring for 30 min, the temperature was raised to rt and the mixture stirred for 2 h. The reaction mixture was condensed under vacuum, and then dispersed into 10 mL of EtOAc, washed with water, evaporated, dried over anhydrous MgSO₄, and evaporated to dryness. The mixture was purified with flash column chromatography using hexanes and ethyl acetate as eluent (2/1, v/v) to produce the titled compound as yellow solids. Yield=19%. ¹H NMR (400 MHz, CDCl₃) δ 9.22 (bs, 1H, NH), 8.10 (bs, 2H, NH2), 7.93 (d, J=1.6 Hz, 1H), 7.89-7.86 (m, 2H), 7.78 (d, J=8.4 Hz, 1H), 7.53 (bs, 1H), 5.16 (d, J=14.8 Hz, 1H), 4.61 (d, J=14.8 Hz, 1H), 4.15 (s, 2H), 1.78 (s, 3H); 19F NMR (CDCl₃, decoupled) δ −62.19. MS (ESI) m/z 452.01 [M+Na]⁺; 428.03 [M−H]⁻.

### (S)—N-(3-((4-Cyano-3-(trifluoromethyl)phenyl)amino)-2-hydroxy-2-methyl-3-oxopropyl)-1H-pyrazole-4-carboxamide (29f)

Compound 29f was prepared following General Procedure A per Scheme 4. The product was purified by a silica gel column using DCM and methanol (19:1) as eluent to afford the titled compound as brown solid. Yield=43%. ¹H NMR (400 MHz, Acetone-d₆) δ 9.92 (bs, 1H, NHCO), 8.44 (d, J=1.8 Hz, 1H), 8.24 (dd, J=8.8, J=1.8 Hz, 1H), 8.12 (s, 1H), 8.03 (d, J=1.8 Hz, 1H), 7.84 (s, 1H), 7.11 (bs, 1H, NHCO), 6.38 (bs, 1H, NH), 5.74 (s, OH), 4.67 (d, J=14.0 Hz, 1H), 4.39 (d, J=14.0 Hz, 1H), 1.50 (s, 3H). ¹⁹F NMR (Acetone-d₆, decoupled) δ 114.69. MS (ESI) m/z 380.1 [M−H]⁻; 382.1 [M+H]⁺. HRMS (ESI) m/z calcd for C₁₆H₁₄F₃NsO₃ 382.1127 [M+H]⁺ found 382.1051 [M+H]⁺; 404.0882 [M+Na]⁺.

### Synthesis of Compound 29g

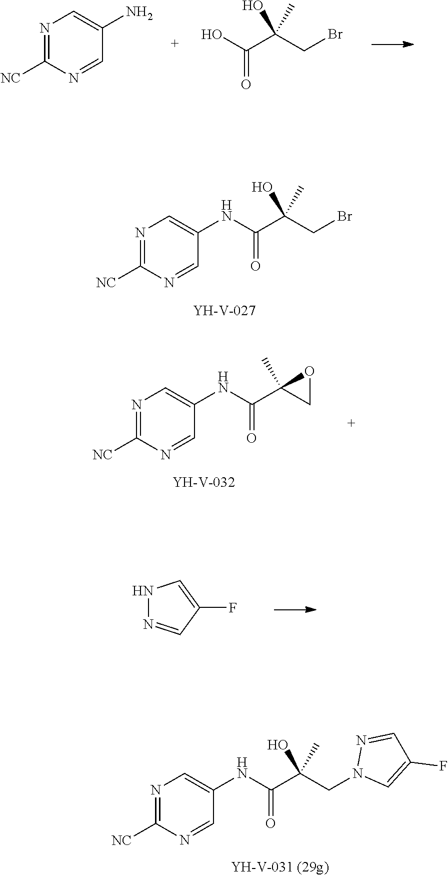

### (R)-3-Bromo-N-(2-Cyanopyrimidin-5-Yl)-2-Hydroxy-2-Methylpropanamide (C₉H9BrN₄O₂)

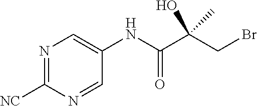 [SMILES: C[C@](O)(CBr)C(=O)Nc1cnc(C#N)nc1]

(R)-3-bromo-2-hydroxy-2-methylpropanoic acid (3.00 g, 0.0163934 mol) reacted with thionyl chloride (2.34 g, 0.01967211 mol), trimethylamine (2.16 g, 0.0213115 mol), and 5-aminopyrimidine-2-carbonitrile (1.97 g, 0.0163934 mol) to afford the title compound. The product was purified by a silica gel column using hexanes and ethyl acetate (1:1) as eluent to afford 3.44 g (73.3%) of the titled compound as yellowish solid.

¹HNMR (400 MHz, DMSO-d₆) δ 10.71 (s, 1H, NH), 9.40-9.37 (m, 2H, ArH), 6.51 (s, 1H, OH), 3.84 (d, J=10.4 Hz, 1H, CH), 3.59 (d, J=10.4 Hz, 1H, CH), 1.50 (s, 3H, CH₃).

Mass (ESI, Positive): [M+H]⁺.

HRMS [C₉H₁₀BrN₄O₂⁺]: calcd 284.9987, found 284.9985 [M+H]⁺. Purity: 97.09% (HPLC).

### (S)—N-(2-Cyanopyrimidin-5-Yl)-2-Methyloxirane-2-Carboxamide (C₉H8N₄O₂)

To a solution of (R)-3-bromo-N-(2-cyanopyrimidin-5-yl)-2-hydroxy-2-methylpropanamide (5.00 g, 0.01754 mol) in 25 mL of 2-butanone, was added potassium carbonate (6.06 g, 0.04384 mol). The resulting reaction mixture was heated at reflux for 2 h under argon atmosphere. After the end of the reaction was established by TLC, the reaction was cooled to room temperature (rt), filtered through a pad of Celite, and rinsed the pad of Celite with 15 mL of 2-butanone. The filtrate was concentrated under vacuum and dried under 25-30 inches vacuum to provide (S)—N-(2-cyanopyrimidin-5-yl)-2-methyloxirane-2-carboxamide.

¹HNMR (400 MHz, DMSO-d₆) δ 10.38 (s, 1H, NH), 9.27 (br. s, 2H, ArH), 3.11 (d, J=5.2 Hz, 1H, CH), 3.07 (d, J=8.8 Hz, 1H, CH), 1.56 (s, 3H, CH₃).

Mass (ESI, Positive): [M+H]⁺.

HRMS [C₉H9N₄O₂⁺]: calcd 205.0726, found 205.0721 [M+H]⁺. Purity: 98.93% (HPLC).

### (S)—N-(2-Cyanopyrimidin-5-Yl)-3-(4-Fluoro-1H-Pyrazol-l-Yl)-2-Hydroxy-2-Methylpropanamide (C₁₂H₁₁FN₆O₂) (29g)

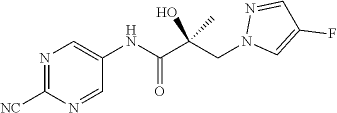 [SMILES: C[C@](O)(Cn1cc(F)cn1)C(=O)Nc1cnc(C#N)nc1]

To a solution of 4-fluoro-1H-pyrazole (0.121 g, 0.001403 mol) in anhydrous THF (10 mL), which was cooled in an ice water bath under an argon atmosphere, was added sodium hydride (60% dispersion in oil, 0.20 g, 0.004910 lmol). After addition, the resulting mixture was stirred for three hours. (R)-3-bromo-N-(2-cyanopyrimidin-5-yl)-2-hydroxy-2-methylpropanamide (0.40 g, 0.001403 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using hexanes and ethyl acetate (1:1) as eluent to afford 0.12g (33.0%) of the titled compound as off-white solid.

¹HNMR (400 MHz, DMSO-d₆) δ 10.53 (s, 1H, NH), 9.30 (br s, 2H, ArH), 7.75 (d, J=4.4 Hz, 1H, pyrazole-H), 7.42 (d, J=4.0 Hz, 1H, pyrazole-H), 6.41 (s, 1H, OH), 4.38 (d, J=14.0 Hz, 1H, CH), 4.19 (d, J=14.0 Hz, 1H, CH), 1.35 (s, 3H, CH₃).

Mass (ESI, Positive): [M+H]⁺.

HRMS [C12H₁₂FN₆O₂⁺]: calcd 291.1006, found 291.1003 [M+H]⁺. Purity: 98.66% (HPLC).

### (S)-3-(4-Cyano-1H-Pyrazol-1-Yl)-N-(2-Cyanopyrimidin-5-Yl)-2-Hydroxy-2-Methylpropanamide (C₁₃H₁₁N₇O₂) (29h)

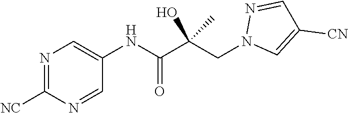 [SMILES: C[C@](O)(Cn1cc(C#N)cn1)C(=O)Nc1cnc(C#N)nc1]

To a solution of 4-cyano-1H-pyrazole (0.131 g, 0.001403 mol) in anhydrous THF (10 mL), which was cooled in an ice water bath under an argon atmosphere, was added sodium hydride (60% dispersion in oil, 0.20 g, 0.0049101 mol). After addition, the resulting mixture was stirred for three hours. (R)-3-bromo-N-(2-cyanopyrimidin-5-yl)-2-hydroxy-2-methylpropanamide (0.40 g, 0.001403 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (9:1) as eluent to afford 0.115 g (27.6%) of the titled compound as yellowish solid.

¹HNMR (400 MHz, DMSO-d₆) δ 10.50 (s, 1H, NH), 9.29 (br s, 2H, ArH), 8.46 (s, 1H, pyrazole-H), 8.00 (s, 1H, pyrazole-H), 6.52 (s, 1H, OH), 4.55 (d, J=14.0 Hz, 1H, CH), 4.37 (d, J=14.0 Hz, 1H, CH), 1.48 (s, 3H, CH₃).

Mass (ESI, Positive): [M+H]⁺.

HRMS [C₁₃H₁₂N₇O₂⁺]: calcd 298.1052, found 298.1055 [M+H]⁺. Purity: 99.26% (HPLC).

### Synthesis of Compound 29i

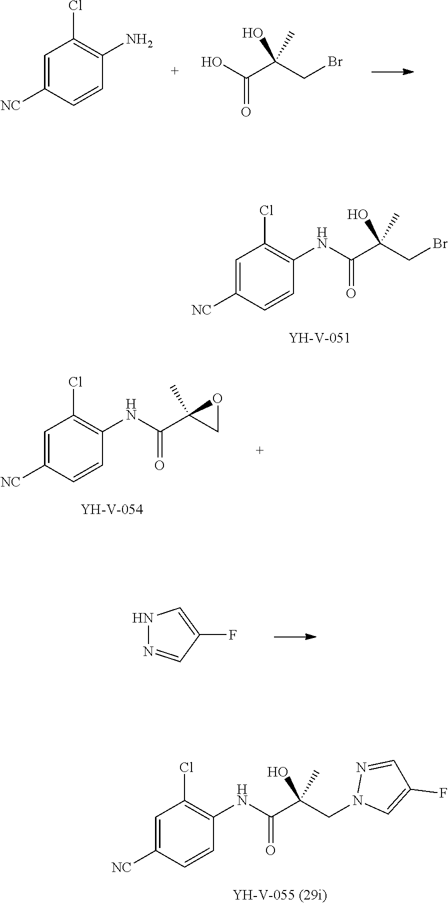

### (R)-3-Bromo-N-(2-Chloro-4-Cyanophenyl)-2-Hydroxy-2-Methylpropanamide (C₁₁H₁₀BrCIN₂O₂)

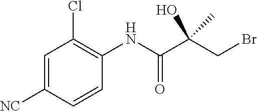 [SMILES: C[C@](O)(CBr)C(=O)Nc1ccc(C#N)cc1Cl]

(R)-3-bromo-2-hydroxy-2-methylpropanoic acid (3.33 g, 0.018182 mol) reacted with thionyl chloride (2.60 g, 0.02182 mol), trimethylamine (2.16 g, 0.0213115 mol), and 5-aminopyrimidine-2-carbonitrile (2.39 g, 0.023638 mol) to afford the title compound. The product was purified by a silica gel column using DCM and ethyl acetate (19:1) as eluent to afford 4.02 g (69.5%) of the titled compound as yellow solid.

¹HNMR (400 MHz, DMSO-d₆) δ 9.78 (s, 1H, NH), 8.49 (dd, J=8.8 Hz, J=4.4 Hz, 1H, ArH), 8.19 (d, J=1.6 Hz, 1H, ArH), 7.88 (dd, J=8.8 Hz, J=2.0 Hz, 1H, ArH), 6.84 (s, 1H, OH), 3.84 (d, J=10.4 Hz, 1H, CH), 3.59 (d, J=10.4 Hz, 1H, CH), 1.56 (s, 3H, CH₃).

Mass (ESI, Positive): [M+H]⁺.

HRMS [C₁₁H₁₁BrClN₂O₂⁺]: calcd 316.9690, found 316.9684 [M+H]⁺. Purity: 98.38% (HPLC).

### (S)—N-(2-Chloro-4-Cyanophenyl)-2-Methyloxirane-2-Carboxamide (C₁₁H9C1N₂O₂)

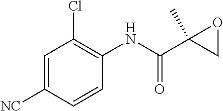 [SMILES: C[C@@]1(C(=O)Nc2ccc(C#N)cc2Cl)CO1]

To a solution of (R)-3-bromo-N-(2-chloro-4-cyanophenyl)-2-hydroxy-2-methylpropanamide (5.00 g, 0.01575 mol) in 25 mL of 2-butanone, was added potassium carbonate (3.26 g, 0.02362 mol). The resulting reaction mixture was heated at reflux for 2 h under argon atmosphere. After the end of the reaction was established by TLC, the reaction was cooled to room temperature (rt), filtered through a pad of Celite, and rinsed the pad of Celite with 15 mL of 2-butanone. The filtrate was concentrated under vacuum and dried under 25-30 inches vacuum to provide (S)—N-(2-chloro-4-cyanophenyl)-2-methyloxirane-2-carboxamide.

¹HNMR (400 MHz, DMSO-d₆) δ 9.17 (s, 1H, NH), 8.19-8.15 (m, 2H, ArH), 7.87-7.84 (m, 1H, ArH), 3.17 (d, J=5.2 Hz, 1H, CH), 3.08 (d, J=5.2 Hz, 1H, CH), 1.56 (s, 3H, CH₃).

Mass (ESI, Positive): [M+H]⁺.

HRMS [C₁₁H₈ClN₂O₂⁻]: calcd 235.0274, found 235.0265 [M+H]⁺. Purity: 67.48% (HPLC).

### (S)—N-(2-Chloro-4-Cyanophenyl)-3-(4-Fluoro-1H-Pyrazol-l-Yl)-2-Hydroxy-2-Methylpropanamide (C₁₄H₁₂ClFN₄O₂) (29i)

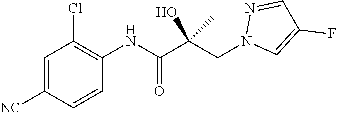 [SMILES: C[C@](O)(Cn1cc(F)cn1)C(=O)Nc1ccc(C#N)cc1Cl]

To a solution of 4-fluoro-1H-pyrazole (0.108 g, 0.0012595 mol) in anhydrous THF (10 mL), which was cooled in an ice water bath under an argon atmosphere, was added sodium hydride (60% dispersion in oil, 0.176 g, 0.0044082 mol). After addition, the resulting mixture was stirred for three hours. (R)-3-bromo-N-(2-chloro-4-cyanophenyl)-2-hydroxy-2-methylpropanamide (0.40 g, 0.0012595 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (19:1 to 9:1) as eluent to afford 0.15 g (31.6%) of the titled compound as off-white solid.

¹HNMR (400 MHz, DMSO-d₆) δ 9.50 (br s, 1H, NH), 8.44 (d, J=8.8 Hz, 1H, ArH), 8.15 (d, J=1.6 Hz, 1H, ArH), 7.86 (dd, J=8.8 Hz, J=2.0 Hz, 1H, ArH), 7.75 (d, J=4.8 Hz, 1H, pyrazole-H), 7.38 (d, J=4.2 Hz, 1H, pyrazole-H), 6.76 (br s, 1H, OH), 4.39 (d, J=14.0 Hz, 1H, CH), 4.12 (d, J=14.0 Hz, 1H, CH), 1.39 (s, 3H, CH3).

Mass (ESI, Positive): [M+H]⁺.

HRMS [C₁₄H₁₃ClFN₄O₂⁺]: calcd 323.0711, found 323.0723 [M+H]⁺.

Purity: 98.81% (HPLC).

### (S)—N-(2-chloro-4-cyanophenyl)-3-(4-cyano-1H-pyrazol-1-yl)-2-hydroxy-2-methylpropanamide (C₁₅H₁₂ClN₅O₂) (29j)

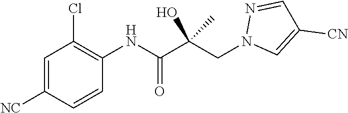 [SMILES: C[C@](O)(Cn1cc(C#N)cn1)C(=O)Nc1ccc(C#N)cc1Cl]

To a solution of 4-cyano-1H-pyrazole (0.117 g, 0.0012595 mol) in anhydrous THF (10 mL), which was cooled in an ice water bath under an argon atmosphere, was added sodium hydride (60% dispersion in oil, 0.176 g, 0.0044082 mol). After addition, the resulting mixture was stirred for three hours. (R)-3-bromo-N-(2-chloro-4-cyanophenyl)-2-hydroxy-2-methylpropanamide (0.40 g, 0.0012595 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (19:1 to 9:1) as eluent to afford 0.15 g (31.6%) of the titled compound as off-white solid.

¹HNMR (400 MHz, DMSO-d₆) δ 9.47 (br s, 1H, NH), 8.46 (s, 1H, pyrazole-H), 8.42 (d, J=8.2 Hz, 1H, ArH), 8.15 (d, J=2.0 Hz, 1H, ArH), 7.97 (s, 1H, pyrazole-H), 7.88 (dd, J=8.2 Hz, 1=2.0 Hz, 1H, ArH), 6.88 (br s, 1H, OH), 4.56 (d, J=14.0 Hz, 1H, CH), 4.36 (d, J=14.0 Hz, 1H, CH), 1.43 (s, 3H, CH₃).

Mass (ESI, Positive): [M+H]⁺.

HRMS [C₁₅H₁₃ClN₅O₂⁺]: calcd 330.0758, found 330.0753 [M+H]⁺. Purity: 95.75% (HPLC).

### Synthesis of Compound 29k

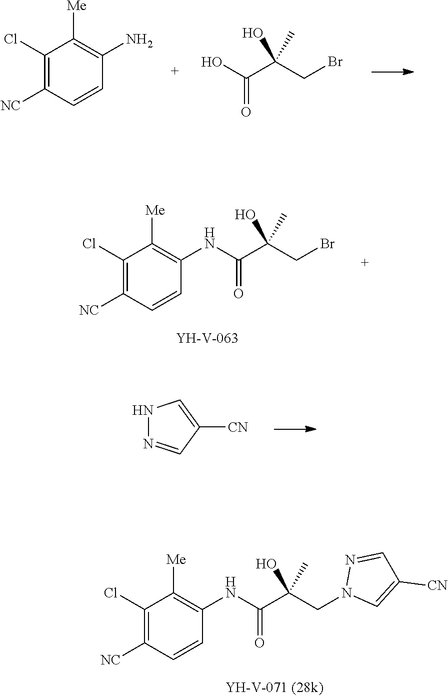

### (R)-3-Bromo-N-(3-Chloro-4-Cyano-2-Methylphenyl)-2-Hydroxy-2-Methylpropanamide (C₁₂H₁₂BrClN₂O₂)

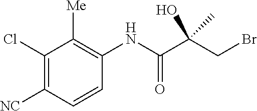 [SMILES: Cc1c(NC(=O)[C@@](C)(O)CBr)ccc(C#N)c1Cl]

(R)-3-bromo-2-hydroxy-2-methylpropanoic acid (1.21 g, 0.006602 mol) reacted with thionyl chloride (0.86 g, 0.007202 mol), trimethylamine (0.79 g, 0.007802 mol), and 4-amino-2-chloro-3-methylbenzonitrile (1.00 g, 0.006002 mol) to afford the title compound. The product was purified by a silica gel column using DCM and ethyl acetate (19:1) as eluent to afford 1.60 g (80.4%) of the titled compound as yellow solid.

¹HNMR (400 MHz, DMSO-d₆) δ 9.70 (s, 1H, NH), 7.84 (d, J=8.4 Hz, 1H, ArH), 7.76 (d, J=8.4 Hz, 1H, ArH), 6.50 (s, 1H, OH), 3.84 (d, J=9.2 Hz, 1H, CH), 3.59 (d, J=9.2 Hz, 1H, CH), 2.32 (s, 3H, CH₃), 1.50 (s, 3H, CH₃).

Mass (ESI, Positive): [M+H]⁺.

HRMS [C₁₂H₁₃BrClN₂O₂⁺]: calcd 330.9849, found 330.9843 [M+H]⁺.

Purity: 98.80% (HPLC).

### (S)—N-(3-Chloro-4-Cyano-2-Methylphenyl)-3-(4-Cyano-1H-Pyrazol-1-Yl)-2-Hydroxy-2-Methylpropanamide (C₁₆H₁₄ClN₅O₂) (29k)

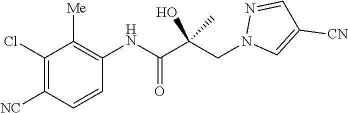 [SMILES: Cc1c(NC(=O)[C@@](C)(O)Cn2cc(C#N)cn2)ccc(C#N)c1Cl]

To a solution of 4-cyano-1H-pyrazole (0.112 g, 0.0012063 mol) in anhydrous THF (10 mL), which was cooled in an ice water bath under an argon atmosphere, was added sodium hydride (60% dispersion in oil, 0.169 g, 0.0042217 mol). After addition, the resulting mixture was stirred for three hours. (R)-3-bromo-N-(3-chloro-4-cyano-2-methylphenyl)-2-hydroxy-2-methylpropanamide (0.40 g, 0.0012063 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (9:1 to 5:1) as eluent to afford 0.31g (74.7%) of the titled compound as off-white solid.

¹HNMR (400 MHz, DMSO-d₆) δ 9.52 (br s, 1H, NH), 8.46 (s, 1H, Pyrazole-H), 8.04 (s, 1H, Pyrazole-H), 7.83 (d, J=8.8 Hz, 1H, ArH), 7.78 (d, J=8.8 Hz, 1H, ArH), 6.51 (br s, 1H, OH), 4.56 (d, J=14.4 Hz, 1H, CH), 4.34 (d, J=14.0 Hz, 1H, CH), 2.18 (s, 3H, CH₃), 1.40 (s, 3H, CH₃).

Mass (ESI, Positive): [M+H]⁺.

HRMS [C₁₆H₁₅ClN₅O₂⁺]: calcd 344.0914, found 344.0910 [M+H]⁺. Purity: 99.59% (HPLC).

### Synthesis of Compounds 291 and 29m

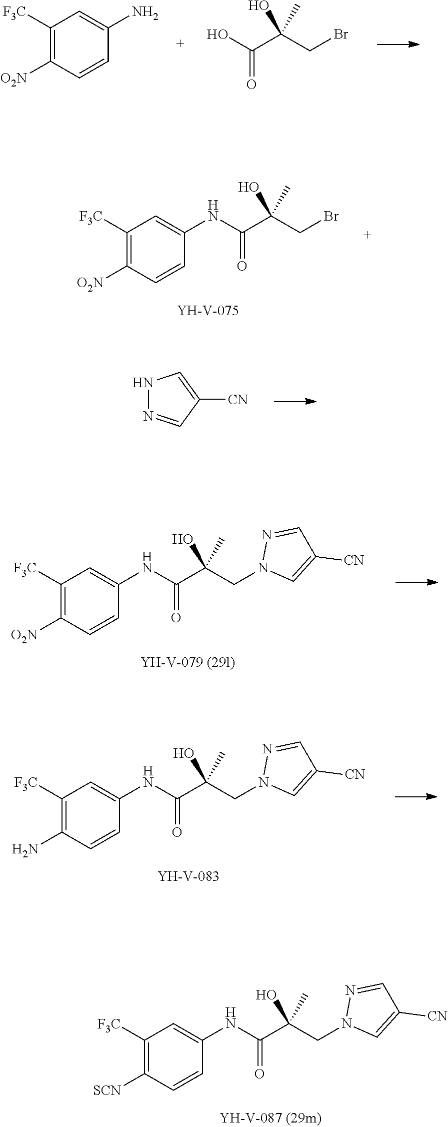

### (R)-3-Bromo-2-Hydroxy-2-Methyl-N-(4-Nitro-3-(Trifluoromethyl)Phenyl)Propanamide (C₁₁H₁₀BrF3N₂O₄)

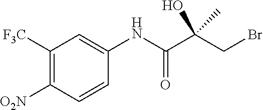 [SMILES: C[C@](O)(CBr)C(=O)Nc1ccc([N+](=O)[O-])c(C(F)(F)F)c1]

(R)-3-bromo-2-hydroxy-2-methylpropanoic acid (1.95 g, 0.0106734 mol) reacted with thionyl chloride (0.385 g, 0.0116437 mol), trimethylamine (1.276 g, 0.012614 mol), and 4-nitro-3-(trifluoromethyl)aniline (2.00 g, 0.0097031 mol) to afford the title compound. The product was purified by a silica gel column using DCM and ethyl acetate (19:1) as eluent to afford 2.70 g (75.0%) of the titled compound as yellow solid.

¹HNMR (400 MHz, DMSO-d₆) δ 10.61 (s, 1H, NH), 8.58 (d, J=2.0 Hz, 1H, ArH), 8.38 (dd, J=8.8 Hz, J=2.0 Hz, 1H, ArH), 8.22 (d, J=8.8 Hz, 1H, ArH), 6.45 (br s, 1H, OH), 3.85 (d, J=10.4 Hz, 1H, CH), 3.61 (d, J=10.4 Hz, 1H, CH), 1.50 (s, 3H, CH₃).

Mass (ESI, Positive): [M+H]⁺.

HRMS [C₁₁H₁₁BrF₃N₂O₄⁺]: calcd 370.9854, found 370.9854 [M+H]⁺.

Purity: 95.23% (HPLC).

### (S)-3-(4-Cyano-1H-Pyrazol-1-Yl)-2-Hydroxy-2-Methyl-N-(4-Nitro-3-(Trifluoromethyl)Phenyl)Propanamide (C₁₅H₁₂F₃NSO₄) (291)

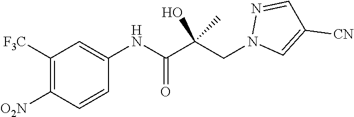 [SMILES: C[C@](O)(Cn1cc(C#N)cn1)C(=O)Nc1ccc([N+](=O)[O-])c(C(F)(F)F)c1]

To a solution of 4-cyano-1H-pyrazole (0.376 g, 0.0040419 mol) in anhydrous THF (20 mL), which was cooled in an ice water bath under an argon atmosphere, was added sodium hydride (60% dispersion in oil, 0.566 g, 0.0141466 mol). After addition, the resulting mixture was stirred for three hours. (R)-3-bromo-2-hydroxy-2-methyl-N-(4-nitro-3-(trifluoromethyl)phenyl)propanamide (1.50 g, 0.0040419 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (9:1 to 5:1) as eluent to afford 0.52g (33.5%) of the titled compound as yellow solid.

¹HNMR (400 MHz, DMSO-d₆) δ 10.42 (br s, 1H, NH), 8.47 (d, J=2.0 Hz, 1H, ArH), 8.46 (s, 1H, Pyrazole-H), 8.30 (dd, J=8.8 Hz, J=2.0 Hz, 1H, ArH), 8.20 (d, J=8.8 Hz, 1H, ArH), 8.00 (s, 1H, Pyrazole-H), 6.44 (br s, 1H, OH), 4.55 (d, J=14.4 Hz, 1H, CH), 4.36 (d, J=14.0 Hz, 1H, CH), 1.39 (s, 3H, CH₃).

Mass (ESI, Positive): [M+H]⁺.

HRMS [C₁₅H₁₃F₃N₅O₄⁺]: calcd 384.0920, found 384.0914 [M+H]⁺. Purity: 100.00% (HPLC).

### (S)—N-(4-Amino-3-(Trifluoromethyl)Phenyl)-3-(4-Cyano-1H-Pyrazol-1-Yl)-2-Hydroxy-2-Methylpropanamide (C₁₅H₁₄F₃N₅O₂)

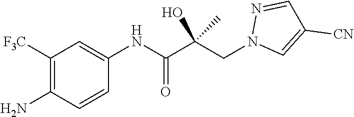 [SMILES: C[C@](O)(Cn1cc(C#N)cn1)C(=O)Nc1ccc(N)c(C(F)(F)F)c1]

(S)-3-(4-cyano-1H-pyrazol-1-yl)-2-hydroxy-2-methyl-N-(4-nitro-3-(trifluoromethyl)phenyl)propenamide (0.30 g, 0.0007827 mol) was hydrogenated on 10% Palladium on charcoal at 25 psi for 2-3 hours at room temperature. After the end of the reaction was established by TLC, the reaction mixture was filtered through Celite, concentrated under vacuum, dried and went to the next step without further purification.

¹HNMR (400 MHz, DMSO-d₆) δ

Mass (ESI, Positive): [M+H]⁺.

HRMS [C₁₅H₁₃F₃N₅O₂]: calcd 352.1021, found 352.1030 [M+H]⁺.

Purity:% (HPLC).

### (S)-3-(4-Cyano-1H-Pyrazol-1-Yl)-2-Hydroxy-N-(4-Isothiocyanato-3-(Trifluoromethyl)Phenyl)-2-Methylpropanamide (C₁₆H₁₂F₃N₅O₂S) (29m)

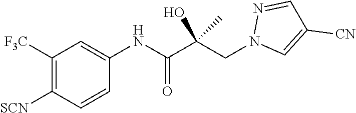

To a solution of (S)—N-(4-amino-3-(trifluoromethyl)phenyl)-3-(4-cyano-1H-pyrazol-lyl)-2-hydroxy-2-methylpropanamide (0.135 g, 0.0003821 mol) in 5 mL of anhydrous THF, which was cooled in an ice-water bath, was added thiophosgene (88 mg, 0.0007642 mol) and triethylamine (0.193 g, 0.0019105 mol) under argon. The resulting reaction mixture was for 4-5 hours at room temperature under argon. The reaction was quenched by water, extracted with ethyl acetate. The organic layer was washed with brine, dried with MgSO₄, filtered, and concentrated under vacuum. The product was purified by a silica gel column using DCM and methanol (9:1) as eluent to afford 20 mg (13.3%) of the titled compound as light brown solid (not very stable).

¹HNMR (400 MHz, DMSO-d₆) δ 10.13 (s, 1H, NH), 8.30 (d, J=2.0 Hz, 1H, ArH), 8.13 (s, 1H, Pyrazole-H), 8.04 (d, J=8.2 Hz, 1H, ArH), 7.64 (dd, J=8.2 Hz, J=2.0 Hz, 1H, ArH), 7.45 (s, 1H, Pyrazole-H), 6.19 (s, 1H, OH), 4.39 (m, 1H, CH), 4.21 (m, 1H, CH), 1.32 (s, 3H, CH₃).

Mass (ESI, Positive): [M+H]⁺.

HRMS [C₁₆H₁₁F₃N₅O═S⁻]: calcd 394.0586, found 396.0613 [M+H]⁺.

Purity: % (HPLC).

### Synthesis of Compound 29q

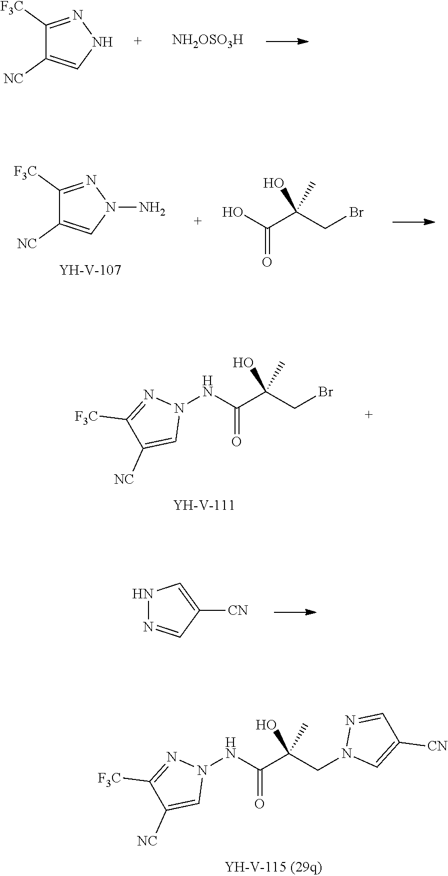

### 1-Amino-3-(Trifluoromethyl)-1H-Pyrazole-4-Carbonitrile (C₅H₃F₃N₄)

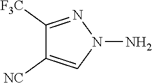 [SMILES: N#Cc1cn(N)nc1C(F)(F)F]

To a solution of 3-(trifluoromethyl)-1H-pyrazole-4-carbonitrile (0.5 g, 0.0031041 mol) in 10 mL of water was added crushed NaOH (0.5 g, 0.012416 mol). The solution was stirred at 55-60° C. for 20 minutes. Hydroxyamine-O-sulfonic acid (1.05 g, 0.009312 mol) was added cautiously by portion to above solution. The resulting reaction mixture was heated at 65° C. for 2 hours and stirred at room temperature for 2 hours. The reaction was extracted with DCM for three times. The organic layer was washed with brine, dried with MgSO₄, filtered, concentrated under vacuum, dried, and went to the next step without further purification.

¹HNMR (400 MHz, DMSO-d₆) δ

HRMS [C₅H2F₃N₄⁻]: calcd 175.0232, found 175.0317 [M−H]⁻. Purity:% (HPLC).

### (R)-3-Bromo-N-(4-Cyano-3-(Trifluoromethyl)-1H-Pyrazol-1-Yl)-2-Hydroxy-2-Methylpropanamide (C₉H₅BRF₃N₄O₂)

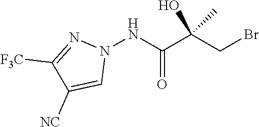 [SMILES: C[C@](O)(CBr)C(=O)Nn1cc(C#N)c(C(F)(F)F)n1]

(R)-3-bromo-2-hydroxy-2-methylpropanoic acid (0.864 g, 0.0047223 mol) reacted with thionyl chloride (0.613 g, 0.0051516 mol), trimethylamine (0.565 g, 0.0055809 mol), and 1-amino-3-(trifluoromethyl)-1H-pyrazole-4-carbonitrile (0.756 g, 0.004293 mol) to afford the title compound. The product was purified by a silica gel column using DCM and ethyl acetate (9:1 to 4:1) as eluent to afford 0.46 g (31.5%) of the titled compound as yellow solid.

### (S)-3-(4-Cyano-1H-Pyrazol-1-Yl)-N-(4-Cyano-3-(Trifluoromethyl)-1H-Pyrazol-1-Yl)-2-Hydroxy-2-Methylpropanamide (C₁₃H₁₀F₃N₁O₂) (29q)

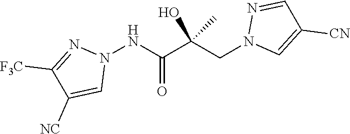 [SMILES: C[C@](O)(Cn1cc(C#N)cn1)C(=O)Nn1cc(C#N)c(C(F)(F)F)n1]

To a solution of 4-cyano-1H-pyrazole (0.15 g, 0.0016183 mol) in anhydrous THF (10 mL), which was cooled in an ice water bath under an argon atmosphere, was added sodium hydride (60% dispersion in oil, 0.19 g, 0.0047201 mol). After addition, the resulting mixture was stirred for two hours. (R)-3-bromo-N-(4-cyano-3-(trifluoromethyl)-1H-pyrazol-1-yl)-2-hydroxy-2-methylpropanamide (0.46 g, 0.0013486 mol) was added to above solution, and the resulting reaction mixture was stirred overnight at room temperature under argon. The reaction was quenched by water, extracted with ethyl acetate. The organic layer was washed with brine, dried with MgSO₄, filtered, and concentrated under vacuum. The product was purified by a silica gel column using DCM and ethyl acetate (4:1 to 2:1) as eluent to afford 0.115 g (50.4%) of the titled compound as yellow solid.

¹HNMR (400 MHz, DMSO-d₆) δ 12.27 (s, 1H, NH), 8.83 (s, 1H, Pyrazole-H), 8.46 (s, 1H, Pyrazole-H), 8.15 (s, 1H, Pyrazole-H), 6.49 (s, 1H, OH), 4.51 (d, J=14.0 Hz, 1H, CH), 4.35 (d, J=14.0 Hz, 1H, CH), 1.40 (s, 3H, CH₃).

Mass (ESI, Positive): [M+H]⁺.

HRMS [C₁₃H9F₃N₇O₂⁻]: calcd 352.0770, found 352.0761 [M−Hr. Purity: 99.00% (HPLC).

### Synthesis of Compound 290

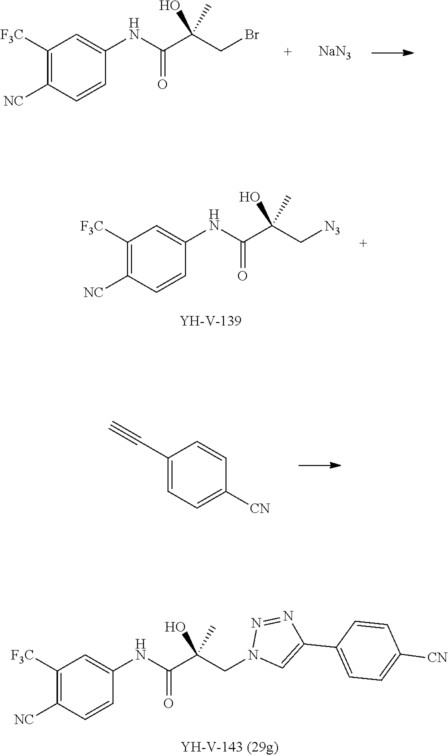

### (S)-3-Azido-N-(4-Cyano-3-(Trifluoromethyl)Phenyl)-2-Hydroxy-2-Methylpropanamide (C₁₂H₁₀F₃N₅O₂)

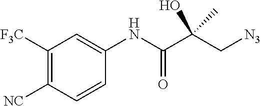 [SMILES: C[C@](O)(CN=[N+]=[N-])C(=O)Nc1ccc(C#N)c(C(F)(F)F)c1]

To a solution of (R)-3-bromo-N-(4-cyano-3-(trifluoromethyl)phenyl)-2-hydroxy-2-methylpropanamide (2.00 g, 0.005696 mol) in anhydrous DMF (10 mL) was added sodium azide (0.74 g, 0.011392 mol). The resulting mixture was heated at 80° C. for 3-4 hours. After the end of reaction was established by TLC, the reaction was quenched by water, extracted with ethyl acetate. The organic layer was washed with brine, dried with MgSO₄, filtered, and reduced volume under vacuum. The product was purified by a silica gel column using DCM and ethyl acetate (9:1) as eluent to afford 0.93g (52.4%) of the titled compound as yellowish solid.

¹HNMR (400 MHz, DMSO-d₆) δ 10.58 (s, 1H, NH), 8.54 (s, 1H, ArH), 8.31 (d, J=8.2 Hz, 1H, ArH), 8.11 (d, J=8.2 Hz, 1H, ArH), 6.43 (s, 1H, OH), 4.02 (d, J=14.0 Hz, 1H, CH), 3.39 (d, J=14.0 Hz, 1H, CH), 1.37 (s, 3H, CH₃).

Mass (ESI, Positive): [M+H]⁺.

HRMS [C₁₂H₁₁F₃N₅O₂⁺]: calcd 314.0865, found 314.0865 [M+H]⁺. Purity: 99.00% (HPLC).

### (S)—N-(4-Cyano-3-(Trifluoromethyl)Phenyl)-3-(4-(4-Cyanophenyl)-1H-1,2,3-Triazol-1-Yl)-2-Hydroxy-2-Methylpropanamide (C₂₁H₁₅F₃N₆O₂) (290)

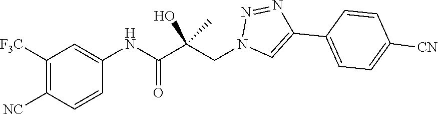 [SMILES: C[C@](O)(Cn1cc(-c2ccc(C#N)cc2)nn1)C(=O)Nc1ccc(C#N)c(C(F)(F)F)c1]

To a solution of (S)-3-azido-N-(4-cyano-3-(trifluoromethyl)phenyl)-2-hydroxy-2-methylpropanamide (0.50 g, 0.0015692 mol) in mixture of CAN and water (8 mL+2 mL) was added 4-ethynylbenzonitrile (0.30 g, 0.0023943 mol), and CuI (30 mg, 0.0001596 mol) as catalyst. The resulting mixture was stirred at room temperature for 3 days (Azide-alkyne Huisgen Cycloaddition, also called Click reaction). The reaction was quenched by water, extracted with ethyl acetate. The organic layer was washed with brine, dried with MgSO₄, filtered, and reduced volume under vacuum. The product was purified by a silica gel column using DCM and methanol (19:1) as eluent to afford 0.22g (31%) of the titled compound as white solid.

¹HNMR (400 MHz, DMSO-d₆) δ 10.44 (s, 1H, NH), 8.63 (s, 1H, Pyrazole-H), 8.42 (s, 1H, ArH), 8.23 (d, J=8.2 Hz, 1H, ArH), 8.09 (d, J=8.2 Hz, 1H, ArH), 8.03 (d, J=8.0 Hz, 2H, ArH), 7.91 (d, J=8.0 Hz, 2H, ArH), 6.56 (s, 1H, OH), 4.79 (d, J=14.0 Hz, 1H, CH), 4.61 (d, J=14.0 Hz, 1H, CH), 1.43 (s, 3H, CH₃).

Mass (ESI, Positive): [M+H]⁺.

HRMS [C₂₁H₁₆F₃N₆O₂⁺]: calcd 441.1287 found 441.1287 [M+H]⁺. Purity:% (HPLC).

### Synthesis of Compound 29p

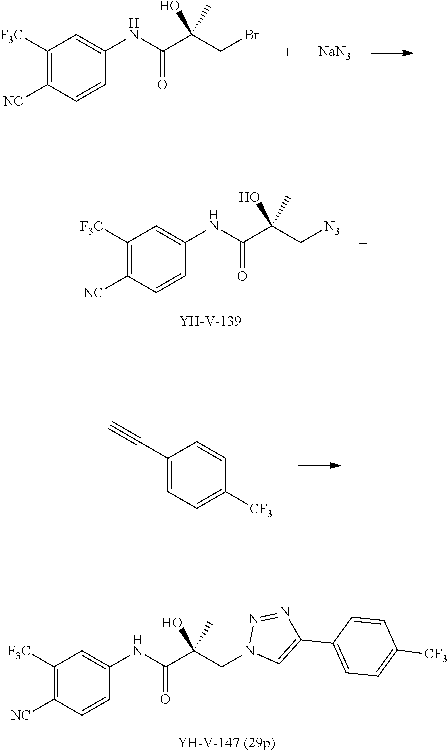 [SMILES: C[C@](O)(CBr)C(=O)Nc1ccc(C#N)c(C(F)(F)F)c1 ; C[C@](O)(CN=[N+]=[N-])C(=O)Nc1ccc(C#N)c(C(F)(F)F)c1 ; C#Cc1ccc(C(F)(F)F)cc1 ; C[C@](O)(Cn1cc(-c2ccc(C(F)(F)F)cc2)nn1)C(=O)Nc1ccc(C#N)c(C(F)(F)F)c1 ; "NaN3"]

### (S)—N-(4-cyano-3-(trifluoromethyl)phenyl)-2-hydroxy-2-methyl-3-(4-(trifluoromethyl)phenyl)-1H-1,2,3-triazol-1-yl)propenamide (C₂₁H₁₅F₆N₅O₂) (29p)

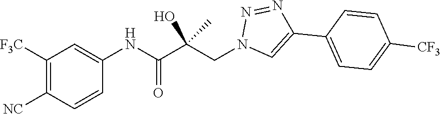 [SMILES: C[C@](O)(Cn1cc(-c2ccc(C(F)(F)F)cc2)nn1)C(=O)Nc1ccc(C#N)c(C(F)(F)F)c1]

To a solution of (S)-3-azido-N-(4-cyano-3-(trifluoromethyl)phenyl)-2-2-hydroxy-2-methylpropanamide (0.50 g, 0.0015692 mol) in mixture of CAN and water (8 mL+2 mL) was added 1-ethynyl-4-(trifluoromethyl)benzene (0.41 g, 0.0023943 mol), and Cul (30 mg, 0.0001596 mol) as catalyst. The resulting mixture was stirred at room temperature for 3 days (Azide-alkyne Huisgen Cycloaddition, also called Click reaction). The reaction was quenched by water, extracted with ethyl acetate. The organic layer was washed with brine, dried with MgSO₄, filtered, and reduced volume under vacuum. The product was purified by a silica gel column using hexanes and ethyl acetate (1:1 to 1:1.5) as eluent to afford 0.538 g (70%) of the titled compound as white solid.

¹HNMR (400 MHz, DMSO-d₆) δ 10.45 (s, 1H, NH), 8.59 (s, 1H, Pyrazole-H), 8.42 (s, 1H, ArH), 8.24 (d, J=8.2 Hz, 1H, ArH), 8.10 (d, J=8.2 Hz, 1H, ArH), 8.05 (d, J=8.0 Hz, 2H, ArH), 7.80 (d, J=8.0 Hz, 2H, ArH), 6.56 (s, 1H, OH), 4.80 (d, J=14.0 Hz, 1H, CH), 4.61 (d, J=14.0 Hz, 1H, CH), 1.44 (s, 3H, CH3).

Mass (ESI, Positive): [M+H]⁺.

HRMS [C₂₁H₁₆F₃N₆O₂⁺]: calcd 441.1287 found 441.1287 [M+H]⁺. Purity:% (HPLC).

### (S)—N-(4-Cyano-3-(trifluoromethyl)phenyl)-2-hydroxy-2-methyl-3-(4-sulfamoyl-1H-pyrazol-1-yl)propenamide (29n)

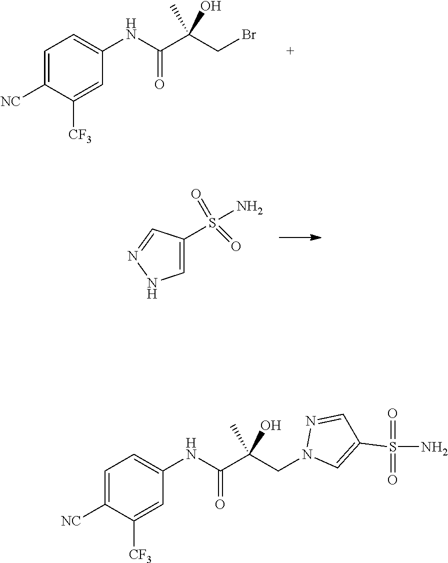 [SMILES: C[C@](O)(CBr)C(=O)Nc1ccc(C#N)c(C(F)(F)F)c1 ; NS(=O)(=O)c1cn[nH]c1 ; C[C@](O)(Cn1cc(S(N)(=O)=O)cn1)C(=O)Nc1ccc(C#N)c(C(F)(F)F)c1]

To a dry, nitrogen-purged 100 mL round-bottom flask equipped a dropping funnel under argon atmosphere, sodium hydride (NaH) of 60% dispersion in mineral oil (240 mg, 6 mmol) was added in 10 mL of anhydrous THF solvent in the flask at ice-water bath, and a solution of 1H-pyrazole-4-sulfonamide (295 mg, 2 mmol) in 5 ml anhydrous THF was added stirred 30 min at the ice-water bath. Into the flask, (R)-3-bromo-N-(4-cyano-3-(trifluoromethyl)phenyl)-2-hydroxy-2-methylpropanamide (702 mg, 2 mmol) in 5 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was crystallized with acetone/hexane to produce the target compound as white solid.

Yield: 48%; Purity: 98.18%; UV max: 270.45; MS (ESI) m/z 416.20 [M−H]⁻; LCMS (ESI) m/z calcd for C₁₅H₁₄F₃N₅O₄S 416.0640 [M−H]⁻; found: 416.0679 [M−H]⁻418.0789 [M+H]⁺, 440.0613 [M+Na]⁺.

¹HNMR (Acetone-d₆, 400 MHz) δ 9.76 (bs, 1H, NH), 8.32 (d, J=1.6 Hz, 1H), 8.11 (dd, J=8.4, 1.6 Hz, 1H), 7.94 (s, 1H), 7.88 (d, J=8.4 Hz, 1H), 6.39 (bs, 2H, SO₂NH₂), 5.53 (bs, OH), 4.54 (d, J=14.4 Hz, 1H), 4.30 (d, J=14.4 Hz, 1H), 1.38 (s, 3H);

¹⁹FNMR (CDCl₃, 400 MHz) δ −62.80.

### Example 2

Androgen Receptor Binding, Transactivation, Degradation, and Metabolism of SARDs Ligand Binding Assay (Ki values)

hAR-LBD (633-919) was cloned into pGex4t.1. Large scale GST-tagged AR-LBD was prepared and purified using a GST column. Recombinant AR-LBD was combined with [3H]mibolerone (PerkinElmer, Waltham, Mass.) in buffer A (10 mM Tris, pH 7.4, 1.5 mM disodium EDTA, 0.25 M sucrose, 10 mM sodium molybdate, 1 mM PMSF) to determine the equilibrium dissociation constant (K_{d}) of [³H]mibolerone. Protein was incubated with increasing concentrations of [3H]mibolerone with and without a high concentration of unlabeled mibolerone at 4° C. for 18 h in order to determine total and non-specific binding. Non-specific binding was then subtracted from total binding to determine specific binding and non-linear regression for ligand binding curve with one site saturation to determine the K_{d} of mibolerone.

Increasing concentrations of SARDs or DHT (range: 10-12 to 10-2 M) were incubated with [3H]mibolerone and AR LBD using the conditions described above. Following incubation, the ligand bound AR-LBD complex was isolated using Bio Gel HT® hydroxyapatite, washed and counted in a scintillation counter after adding scintillation cocktail. Values are expressed as Kᵢ.

Transactivation Assay with Wt AR (IC₅₀ Values)

HEK-293 cells were plated at 125,000 cells/well of a 24 well plate in DME+5% csFBS without phenol red. Cells were transfected with 0.25 ug GRE-LUC, 10 ng CMV-renilla LUC, and 50 ng CMV-hAR(wt) using Lipofectamine transfection reagent in optiMEM medium. Medium was changed 24 h after transfection to DME+5% csFBS without phenol red and treated with a dose response of various drugs (1 pM to 10 μM). SARDs and antagonists were treated in combination with 0.1 nM R1881. Luciferase assay was performed 24 h after treatment on a Biotek synergy 4 plate reader. Firefly luciferase values were normalized to renilla luciferase values.

### Plasmid Constructs and Transient Transfection

Human AR cloned into CMV vector backbone was used for the transactivation study. HEK-293 cells were plated at 120,000 cells per well of a 24 well plate in DME+5% csFBS. The cells were transfected using Lipofectamine (Invitrogen, Carlsbad, Calif.) with 0.25 μg GRE-LUC, 0.01 μg CMV-LUC (renilla luciferase) and 25 ng of the AR. The cells were treated 24 hrs after transfection as indicated in the figures and the luciferase assay performed 48 hrs after transfection. Data are represented as IC₅₀ obtained from four parameter logistics curve.

### LNCaP Gene Expression Assay

LNCaP cells were plated at 15,000 cells/well of a 96 well plate in RPMI+1% csFBS without phenol red. Forty-eight hours after plating, cells were treated with a dose response of SARDs. Twenty four hours after treatment, RNA was isolated using cells-to-ct reagent, cDNA synthesized, and expression of various genes was measured by realtime rtPCR (ABI 7900) using taqman primers and probes. Gene expression results were normalized to GAPDH.

### LNCaP Growth Assay

LNCaP cells were plated at 10,000 cells/well of a 96 well plate in RPMI+1% csFBS without phenol red. Cells were treated with a dose response of SARDs. Three days after treatment, cells were treated again. Six days after treatment, cells were fixed and cell viability was measured by SRB assay.

### LNCaP or AD1 Degradation (AR FL)

LNCaP or AD1 cells expressing full length AR were plated at 750,000-1,000,000 cells/well of a 6 well plate in growth medium (RPMI+10% FBS). Twenty four hours after plating, medium was changed to RPMI+1% csFBS without phenol red and maintained in this medium for 2 days. Medium was again changed to RPMI+1% csFBS without phenol red and cells were treated with SARDs (1 nM to 10 μM) in combination with 0.1 nM R1881. After 24 h of treatment, cells were washed with cold PBS and harvested. Protein was extracted using salt-containing lysis buffer with three free-thaw cycles. Protein concentration was estimated and five microgram of total protein was loaded on a SDS-PAGE, fractionated, and transferred to a PVDF membrane. The membrane was probed with AR N-20 antibody from SantaCruz and actin antibody from Sigma.

### 22RV1 and D567es Degradation (AR SV)

22RV1 and D567es cells expressing AR splice variants were plated at 750,000-1,000,000 cells/well of a 6 well plate in growth medium (RPMI+10% FBS). Twenty four hours after plating, medium was changed and treated. After 24-30 h of treatment, cells were washed with cold PBS and harvested. Protein was extracted using salt-containing lysis buffer with three free-thaw cycles. Protein concentration was estimated and five microgram of total protein was loaded on a SDS-PAGE, fractionated, and transferred to a PVDF membrane. The membrane was probed with AR N-20 antibody from SantaCruz and actin antibody from Sigma.

### 22RV1 Growth and Gene Expression

Cell growth was evaluated as described before by SRB assay. Cells were plated in a 96 well plate in full serum and treated for 6 days with medium change after day 3. Gene expression studies were performed in 22RV1 cells plated in 96 well plate at 10,000 cells/well in RPMI+10% FBS. Twenty four hours after plating, cells were treated for 3 days and gene expression studies were performed as described before.

### Transactivation (ICSO)

In vitro AR antagonism of indicated compounds in Table A. COS7 cells were transfected with 0.25 ug GRE-LUC, 0.01 ug CMV-renilla LUC, and 25 ng CMV-hAR using lipofectamine in optiMEM medium. Cells were treated 24 hours after transfection in the presence of 0.1 nM R1881 and luciferase assay performed 48 hours after transfection. Firefly luciferase values were normalized to renilla luciferase values.

### Degradation

Table A presents FL and SV AR degradation activity for indicated compounds. The numbers under each column represents the % change from vehicle. The bands were quantified using Image software. For each value, the AR band was divided by GAPDH band and the % difference from vehicle was calculated and represented. The numbers shown are O (no degradation) or represented as decreases in AR levels normalized for GAPDH levels. For FL AR degradation, LNCaP cells were maintained in charcoal-stripped PBS-containing medium for 2 days. Cells were treated in this medium in the presence of 0.1 nM R1 881. Cells were harvested 24 hours after treatment, protein extracted, and Western blot for AR and GAPDH was performed. For SV AR degradation, 22RV1 cells were treated as indicated for LNCaP.

### Determination of Metabolic Stability (In Vitro CLᵢₙₜ) of Test Compounds

### Phase I Metabolism

The assay was done in a final volume of 0.5 ml in duplicates (n=2). Test compound (1 μM) was pre-incubated for 10 minutes at 37° C. in 100 mM Tris-HCl, pH 7.5 containing 0.5 mg/ml liver microsomal protein. After pre-incubation, reaction was started by addition of 1 mM NADPH (pre-incubated at 37° C.). Incubations were carried out in triplicate and at various timepoints (0, 5, 10, 15, 30 and 60 minutes) 100 μL aliquots were removed and quenched with 100 μL of acetonitrile containing internal standard. Samples were vortex mixed and centrifuged at 4000 rpm for 10 minutes. The supernatants were transferred to 96 well plates and submitted for LC-MS/MS analysis. As control, sample incubations done in absence of NADPH were included. From % PCR (% Parent Compound Remaining), rate of compound disappearance is determined (slope) and in vitro CLmt (L/min/mg protein) was calculated.

### Metabolic Stability in Phase I & Phase II Pathways

In this assay, test compound was incubated with liver microsomes and disappearance of drug was determined using discovery grade LC-MS/MS. To stimulate Phase II metabolic pathway (glucuronidation), UDPGA and alamethicin was included in the assay.

### LC-MS/MS Analysis

The analysis of the compounds under investigation was performed using LC-MS/MS system consisting of Agilent 1100 HPLC with an MDS/Sciex 4000 Q-Trap™ mass spectrometer. The separation was achieved using a Cis analytical column (Alltima™, 2.1×100 mm, 3 m) protected by a Cis guard cartridge system (SecurityGuard™ ULTRA Cartridges UHPLC for 4.6 mm ID columns, Phenomenex). Mobile phase was consisting of channel A (95% acetonitrile+5% water+0.1% formic acid) and channel C (95% water+5% acetonitrile+0.1% formic acid) and was delivered at a flow rate of 0.4 mL/min. The volume ratio of acetonitrile and water was optimized for each of the analytes. Multiple reaction monitoring (MRM) scans were made with curtain gas, collision gas, nebulizer gas, and auxiliary gas optimized for each compound, and source temperature at 550° C. Molecular ions were formed using an ion spray voltage of −4200 V (negative mode). Declustering potential, entrance potential, collision energy, product ion mass, and cell exit potential were optimized for each compound.

### LC-MS/MS Analysis for Determining Rat Serum Concentrations

Serum was collected 24-30 hours after last dose. 100 μL of serum was mixed with 200 μL of acetonitrile/internal standard. Standard curves were prepared by serial dilution of standards in nM with 100 μL of rat serum, concentrations were 1000, 500, 250, 125, 62.5, 31.2, 15.6, 7.8, 3.9, 1.9, 0.97, and 0. Standards were with extracted with 200 μL of acetonitrile/internal standard. The internal standard for these experiments was (S)-3-(4-cyanophenoxy)-N-(3-(chloro)-4-cyanophenyl)-2-hydroxy-2-methylpropanamide.

The instrumental analysis of the analyte SARD was performed using LC-MS/MS system consisting of Agilent 1100 HPLC with an MDS/Sciex 4000 Q-Trap™ mass spectrometer. The separation was achieved using a C18 analytical column (Alltima™, 2.1×100 mm, 3 m) protected by a C18 guard column (Phenomenex™ 4.6 mm ID cartridge with holder). Mobile phase was consisting of channel A (95% acetonitrile+5% water+0.1% formic acid) and channel C (95% water+5% acetonitrile+0.1% formic acid) and was delivered isocratically at a flow rate of 0.4 mL/min at 70% A and 30% B. The total runtime for analyte SARD was optimized but generally 2-4 minutes, and the volume injected was 10 μL. Multiple reaction monitoring (MRM) scans were made with curtain gas at 10; collision gas at medium; nebulizer gas at 60.0 and auxiliary gas at 60.0 and source temperature at 550° C. Molecular ions were formed using an ion spray voltage (IS) of 4200 (negative mode). Declustering potential (DP), entrance potential (EP), collision energy (CE), product ion mass, and cell exit potential (CXP) were optimized for each analyte SARD for the mass pair observed.

### Log P: Octanol-Water Partition Coefficient (Log P)

Log P is the log of the octanol-water partition coefficient, commonly used early in drug discovery efforts as a rough estimate of whether a particular molecule is likely to cross biological membranes. Log P was calculated using ChemDraw Ultra version is 12.0.2.1016 (Perkin-Elmer, Waltham, Mass. 02451). Calculated Log P values are reported in Table A in the column labeled ‘Log P (−0.4 to +5.6)’. Lipinski's rule of five is a set of criteria intended to predict oral bioavailability. One of these criteria for oral bioavailability is that the Log P is between the values shown in the column heading (−0.4 (relatively hydrophilic) to +5.6 (relatively lipophilic) range), or more generally stated <5. One of the goals of SARD design was to improve water solubility. The monocyclic templates of this invention such as the pyrazoles, pyrroles, etc. were more water soluble than earlier analogs.

TABLE A
In vitro screening of LBD binding (Ki), AR antagonism (IC₅₀), SARD
activity, and metabolic stability

| | | | | | | SARD |
|---|---|---|---|---|---|---|
| | | | | | | Activity |
| | | | | | | Full Length |
| | | Log P | | Binding/Wt. | | % |
| Compd | | (−0.4 | | Ki (nM) | | degradation |
| ID | | to | | (DHT = | IC₅₀ | at 1.10 |
| (Scaffold) | Structure | +5.6) | M.W. | 1 nM) | (nM) | μM |
| 29g | 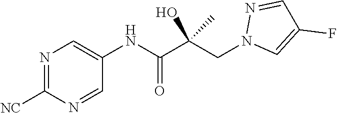 [SMILES: C[C@](O)(Cn1cc(F)cn1)C(=O)Nc1cnc(C#N)nc1] | 0.06 | 290.25 | | — | |
| 29h | 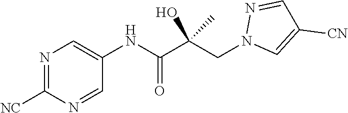 [SMILES: C[C@](O)(Cn1cc(C#N)cn1)C(=O)Nc1cnc(C#N)nc1] | −0.06 | 297.27 | | — | |
| 29i | 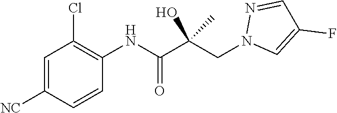 [SMILES: C[C@](O)(Cn1cc(F)cn1)C(=O)Nc1ccc(C#N)cc1Cl] | 1.66 | 322.72 | | 1232 | |
| 29j | 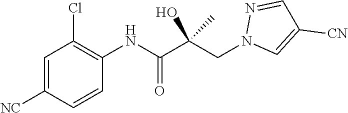 [SMILES: C[C@](O)(Cn1cc(C#N)cn1)C(=O)Nc1ccc(C#N)cc1Cl] | 1.54 | 329.74 | | 1123 | |
| 29k | 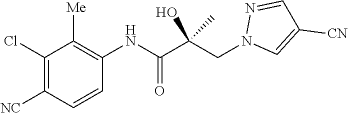 [SMILES: Cc1c(NC(=O)[C@@](C)(O)Cn2cc(C#N)cn2)ccc(C#N)c1Cl] | 2.03 | 343.77 | | 1700 | |
| 29l | 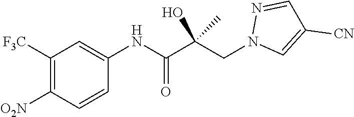 [SMILES: C[C@](O)(Cn1cc(C#N)cn1)C(=O)Nc1ccc([N+](=O)[O-])c(C(F)(F)F)c1] | 1.90 | 383.28 | No binding | 29.2 | 15 |
| 29m | 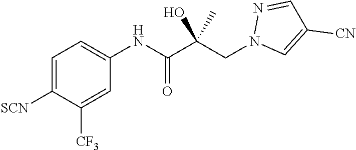 | 2.03 | 395.36 | | — | |
| 29n | 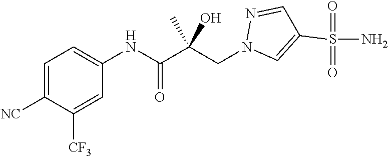 [SMILES: C[C@](O)(Cn1cc(S(N)(=O)=O)cn1)C(=O)Nc1ccc(C#N)c(C(F)(F)F)c1] | 0.61 | 417.36 | | 11.500 | 22 |
| 29o | 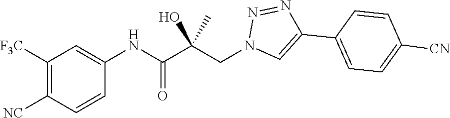 [SMILES: C[C@](O)(Cn1cc(-c2ccc(C#N)cc2)nn1)C(=O)Nc1ccc(C#N)c(C(F)(F)F)c1] | 3.61 | 440.38 | 1514 | 62 | |
| 29p | 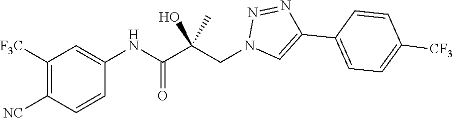 [SMILES: C[C@](O)(Cn1cc(-c2ccc(C(F)(F)F)cc2)nn1)C(=O)Nc1ccc(C#N)c(C(F)(F)F)c1] | 4.50 | 483.37 | 1892 | 214 | |
| 29q | 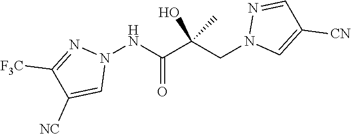 [SMILES: C[C@](O)(Cn1cc(C#N)cn1)C(=O)Nn1cc(C#N)c(C(F)(F)F)n1] | | 353.26 | | — | |

### Example 3: Monosubstitutions of Pyrazole Moiety (B-Ring, Series I)

### Biological Method

### Competitive Ligand-Binding Assay

AR ligand-binding assay was performed as described previously using purified ARLBD cloned from rat prostate (Cancer Res 2017, 77, 6282-6298; J Med Chem 2019, 62, 491-511).

### AR Transactivation Assay

HEK-293 cells plated in 24 well plates at 70,000 cells/well were transfected using lipofectamine transfection reagent (Life Technologies, Carlsbad, Calif.). Cells were transfected with 0.25 μg GRE-LUC, 25 ng CMV-hAR, and 10 ng CMV-LUC. Cells were treated 24 hours after transfection and luciferase assay performed 48 hours after transfection. Firefly luciferase assay values were normalized to Renilla luciferase assay numbers.

### Mutant AR (F876L) and Wt PR Transactivation Assay

COS cells were plated at 70,000 cells/well of a 24 well plate in DME plus 5% csFBS without phenol red. Cells were transfected with 0.25 μg GRE-LUC, 10 ng CMV-renilla LUC, and 50 ng pCR3.1-hPR(wt) or F876L AR using Lipofectamine transfection reagent in optiMEM medium. Medium was changed 24 h after transfection to DME+5% csFBS without phenol red and treated with a dose response of various drugs (1 pM to 10 μM) in the presence or absence of 0.1 nM progesterone (PR) or R1881 (F876L AR). Luciferase assay was performed 24 h after treatment on a Biotek synergy 4 plate reader. Firefly luciferase values were normalized to renilla luciferase values.

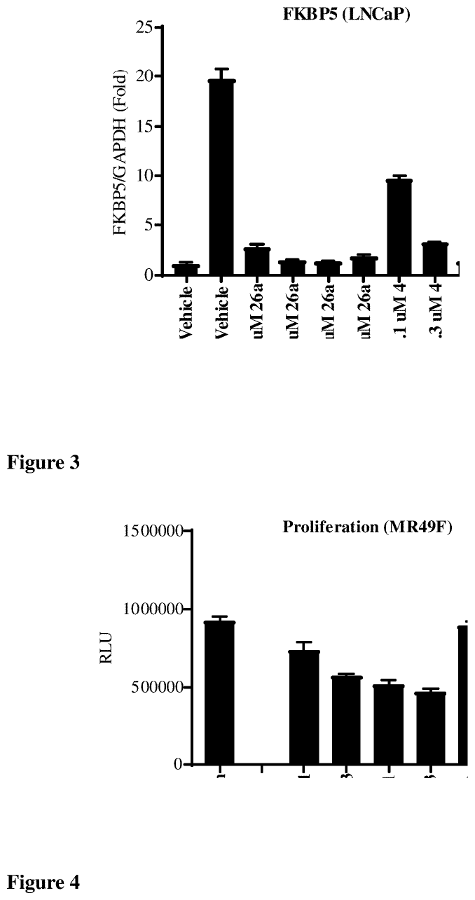

### Androgen Receptor Dependent Gene Expression in LNCaP Cells (FIG. 3)

LNCaP cells were plated in 96 well plates in RPMI plus 1% csFBS without phenol red. Cells were maintained in this medium for two days and treated in the presence of 0.1 nM R1 881. Twenty-four hours after treatment, the cells were harvested, RNA was isolated, and cDNA was prepared using cells-to-ct kit (Life Technologies). Expression of genes was measured using realtime PCR using TaqMan primers and probe (Life Technologies).

### Cellular Proliferation Assays in MR49F LNCaP Cells (FIG. 4)

MR49F cells were plated in 96 well plates in RPMI plus 1% csFBS without phenol red. Cells were treated in this medium in the presence of 0.1 nM R1881 for six days, with medium change and retreatment after three days. Number of viable cells was measured using cell-titer-glo (Promega).

### Western Blot

Indicated cell lines were treated for 24 hours. Cells were harvested, protein extracted and Western blot for AR, AR-SV, and GAPDH was performed using AR PG-21 rabbit poly clonal antibody that binds to the N-terminus of the AR.1, 12

### In Vitro Metabolism Assays

DMPK assays were performed as described before. ^{1, 12} Metabolism assays were performed in mouse, rat, and human liver microsomes as described before.

### In Vivo Pharmacokinetics in Rats

PK studies were conducted at Covance using standard methods as briefly discussed below.

### Animal Husbandry and Experimental Design

Male Sprague Dawley rats from Envigo RMS, Inc. were acclimated to study conditions for 5 days prior to initial dose administration. At initial dosing, the animals were 12 weeks of age. The animals were group housed (up to three animals/cage/group) in polycarbonate cages with hardwood chip bedding. Certified Rodent Diet #2016C (Envigo RMS, Inc.) was provided ad libitum. Water was provided fresh daily, ad libitum. Environmental controls for the animal room were set to maintain a temperature of 20 to 26° C., a relative humidity of 50±20%, and a 12-hour light/12-hour dark cycle. As necessary, the 12-hour dark cycle was interrupted to accommodate study procedures. The test article was prepared in 15% dimethyl sulfoxide (DMSO)/85% polyethylene glycol (PEG) 300 by Covance. Individual doses were calculated based on body weights recorded on Day 1 and Day 7 of dose administration. A single oral daily dose was administered via a gavage needle on seven consecutive days, and blood was sampled as described below. A single intravenous dose was administered via a tail vein and blood sample on Day 1.

Additional detailed information for the 26a experiment, including the groups, number of animals per group, dose (oral 5, 10, 20 and 30 mg/kg per day for seven days; iv 10 mg/kg on Day 1), and route are given in the Experimental Design Table B below. Animals were observed for mortality and signs of pain and distress twice daily (a.m. and p.m.), and cage side observations for general health and appearance were done once daily. Animals were weighed at the time of animal selection and on Day 1 and Day 7 of dose administration.

TABLE B
Experimental design of seven day rat pharmacokinetics experiment

| Group Designations | | | | | Target | |
|---|---|---|---|---|---|---|
| and Dose Levels | | | | Target | Dose | Target |
| | Number | | | Dose | Concen- | Dose |
| | of | Test | Dose | Level | tration | Volume |
| Group | Animals | Article | Route | (mg/kg/day) | (mg/mL) | (mL/kg) |
| 1 | 6 Males | 26a | Oral ^{a} | 5 | 1.67 | 3 |
| 2 | 6 Males | 26a | Oral ^{a} | 10 | 3.33 | 3 |
| 3 | 6 Males | 26a | Oral ^{a} | 20 | 6.67 | 3 |
| 4 | 6 Males | 26a | Oral ^{a} | 30 | 10 | 3 |
| 5 | 6 Males | 26a | IV ^{b} | 10 | 10 | 1 |

IV—intravenous; given as a bolus injection.
^{a} Animals received a single daily dose for seven consecutive days.
^{b} Animals received a single bolus intravenous injection on Day 1 only.

### Sample Collection

Blood (approximately 0.5 mL) was collected via a jugular vein via syringe and needle and transferred into tubes containing K₃EDTA on Days 1 and 7 from three animals/group predose (Day 7 only) and at approximately 0.083, 0.25, 0.5, 1, 3, 6, 12, and 24 h post dose. For i.v. group, blood (approximately 0.5 mL) was collected via a jugular vein at approximately 0.083, 0.25, 0.5, 1, 3, 6, 12, and 24 h postdose. Blood was maintained in chilled cryoracks prior to centrifugation to obtain plasma. Centrifugation began within 1 hour of collection. Plasma was placed into 96-well tubes with barcode labels. Plasma was maintained on dry ice prior to storage at approximately −70° C. Drug concentrations were measured by established chromatography/mass spectrometry (LC-MS/MS) methods.

### Hershberger Assay

Male rats (6-8 weeks old) were randomized into groups based on body weight. Animals were treated with drugs by oral administration as indicated in the figures for 14 days. Animals were sacrificed, prostate and seminal vesicles were weighed, and organ weights were normalized to body weight. Male rats (n=5/group) were left intact, for 13 days. Intact rats were treated with the indicated compounds at the indicated dose by mouth daily for 13 days. Rats were sacrificed on day 14 of treatment and prostate and seminal vesicles organs were removed and weighed. Organ weights were normalized to body weight. This 20 mg/kg fixed dose screening Hershberger which was performed for 10, 21a, 16i (toxic so no data), and 26a. The goal of the experiments was to find compounds with in vivo antiandrogen efficacies greater than 10.

### Xenograft Studies

Xenograft studies were performed at Hera Biolabs (Lexington, Ky.). Enzalutamide resistant VCaP (MDVR VCaP; licensed from Dr. Donald McDonnell, Duke University, Durham, N.C.) cells were implanted subcutaneously in SRG rats (n=5-7/group) (Hera Biolabs). Once the tumors grow to 1000-2000 mm³, the animals were randomized and treated with the indicated drugs. Tumor volume was measured thrice weekly. Thirty days after treatment, the animals were sacrificed, tumors were weighed, and stored for further analysis.

The syntheses of 16a-16x were performed according to Scheme 1. Commercially available (R)-3-bromo-2-hydroxy-2-methylpropanoic acid (11) was treated with SOCl₂ to transform the acid 11 to acid chloride (R)-3-bromo-2-hydroxy-2-methylpropanoyl chloride (not shown), which reacted with aniline (12) to afford the bromide compound (13). Under basic conditions (e.g., K₂CO₃), 13 was transformed to a key oxirane intermediate (14). Alkylation of commercially available pyrazoles (15) by their reaction with 14 afforded the pyrazol-1-ylpropanamides 16a-16x. Series I and all other compounds tested herein were screened in vitro for AR LBD binding (Ki), inhibition of transactivation (IC₅₀), AR degradation (% degradation) of full length (AR FL in LNCaP cells) and splice variant (AR SV in 22RV1 cells) androgen receptors in prostate cancer cell lines, and degradation potency (DC₅₀ values) in LNCaP cells (Table 2). Optimal SARDs and pan-antagonists are compounds that potently inhibit AR transactivation (IC₅₀) and optionally degrade AR FL or AR SV and possess in vivo efficacy in models of antiandrogen resistant CRPC of greater potency than 10.

Compound 16a, which has no substitution on the pyrazole ring, possessed weak AR inhibitory activity with an IC₅₀ value of 1.442 μM. AR inhibition in vitro is defined as the ability to inhibit R1881-induced wtAR transcriptional activity as measured by the luciferase assay [see values in the Transactivation (IC₅₀) column of Table 2], referred to as in vitro AR inhibition herein. Introducing a halogen on the pyrazole significantly increased AR inhibitory activity, except for the 4-iodo compound 16e. The order of AR inhibitory potency with halogen substitution was: 16c (4-Cl, 0.136 μM)>10 (4-F, 0.199 μM)>16b (3-F, 0.220 μM)>16d (4-Br, 0.427 μM)>16a (4-H, 1.442 μM)>16e (4-1, 2.038 μM). Compounds with 4-substitution exhibited more potent AR inhibitory activity than that of their 3-substitution counterparts, for example, comparing 10 (4-F) to 16b (3-F), 16g (4-CF3) to 16h (3-CF3), 16m (4-phenyl) to 16n (3-phenyl), and 160 [4-(4-fluorophenyl)] to 16p [3-(4-fluorophenyl)], respectively.

The stronger the electron withdrawing group (EWGs) is on the pyrazole ring, the more potent is the AR inhibitory activity, with the potency order of 16j (4-NO2, 0.036 μM)>16i (4-CN, 0.045 μM)>16g (4-CF₃, 0.071 μM)>16h (3-CF₃, 0.205 μM)>16q (4-ethynyl, 0.276 μM)>16f (4-COCH₃, 0.758 μM). Compounds bearing an electron donating group on the pyrazole ring showed low potency AR inhibitory activity (16l, 16u, and 16x), no AR inhibitory activity (16k, 16s, 16v, and 16w), or even AR agonist activity (16t which is 4-NH₂). For example, 16r bearing [4-(4-OH-but-1-yn-1-yl)] on the pyrazole ring exhibited no AR inhibitory activity but showed 51% AR full length protein degradation activity.

With regard to SARD activity, substitution of the pyrazole ring seems to be necessary (16a; 0%/0% in % degradation) but some electron donating groups such as 4-OCH₃ of 16k and 4-phenyl or 3-phenyl in 16m and 16n are inactive. Like AR inhibitory potency, the strength of the electron withdrawing groups (EWGs) and 4-substitution seem to contribute favorably as seen in 10 (4-F; 100%/100% for AR FL and AR SV efficacies), 16g (4-CF₃; 80%/100% efficacies), 16i (4-CN; 90%/100% efficacy), whereas 3-substituted EWGs possessed slightly lower SARD activity as can be seen in 16b (3-F; 82%/73% efficacy) and 16h (3-CF₃; 67%/54% efficacy). However, 16p (3-(4-fluorophenyl)) is superior to its 4-position isomer 16o (4-(4-fluorophenyl)) with 54%/81% vs 72%/0% degradation efficacies.

Inhibitory potency (IC₅₀) does not always correlate with % degradation. For example, the most potent inhibitor 16j (4-NO₂; 0.036 μM) was a poor degrader, and the most potent Series I halogen 16c (4-Cl; 0.136 μM) demonstrated only moderate SARD activity (71%/34%). Further, LBD binding (Ki) does not correlate with AR inhibitory potency (IC₅₀) or SARD activity. For example, non-binders 10 and 16c (Kᵢ values>10 μM) inhibited and degraded, whereas nonbinder 16r degraded but was not an inhibitor.

The structure-activity relationships (SARs) of % efficacy of SARD activity (considering AR FL SARD and AR SV SARD activity in aggregate due to their limitation as semi-quantitative values) seems to correlate with the AR inhibitory potency to some degree and LBD Kᵢ to a [much] lesser degree. The screening profile is intended to allow to maximize FL and SV SARD efficacies and AR inhibitory potency to provide the most potent and broad scope antagonists for testing in the models of antiandrogen resistance CRPC. In some cases, such as 16g, 16i, and 26a, high efficacy SARDs (>70% for both AR FL and AR SV) are potent AR inhibitors (<0.100 μM IC₅₀); and moderate efficacy SARDs were moderate potency inhibitors such as 16b, 16c, 16h, and 26c. However, % SARD efficacy does not always correlate well with in vitro inhibitory potency or LBD binding. For example, 16j was a poor degrader, possessing only 20% AR FL efficacy (N.A. for SV), but very potent inhibition (0.036 μM) compared to LBD binding (2.225 μM), and 26f, which possessed only 8%, 15% AR FL efficacy at 1 and 10 μM but extremely potent inhibition (0.035 μM), which is >10-fold more potent than LBD binding (0.567 μM). Thus, it has been considered to place primary emphasis of these molecules as AR degraders (i.e., SARDs) instead of placing emphasis of their ability to inhibit, and in most cases degrade, all AR forms tested to date. In an effort to determine the contribution of AR SARD activity to the AR antagonism observed, the degradation potency values (DC₅₀ values) in LNCaP cells have been provided here for the first time. These values were approximately 4- to 10-fold greater than IC₅₀ values (Tables 2-5), suggesting that SARD activity alone may not explain the potent AR pan-antagonism of these compounds. Hence, these broad scopes and potent noncanonical AR antagonists are noted as SARDs and pan-antagonists herein.

TABLE 2
In Vitro AR Activity of 16a-16x (Series I) and
Approved Antiandrogens

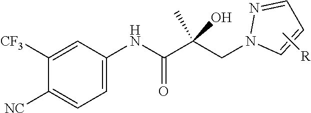

| | Binding (Ki)/ | | | | |
|---|---|---|---|---|---|
| | Trans- | | SARD Activity | | |
| | activation(IC₅₀) | | (% degradation) | | |
| | (μM) | | Full | Splice | |
| | Kᵢ | | Length^{c} | Variant^{c} | F.L. |
| Compound ID | (DHT = | | (LNCaP) | (22RV1) | DC₅₀ |
| (R-group) | 1 nM)^{a} | IC₅₀^{b} | at 1 μM | at 10 μM | (μM) |
| 16a (4-H) | 7.398 | 1.442 | 0 | 0 | |
| 10 (4-F)^{d} | >10 | 0.199 | 100 | 100 | 0.74 |
| 16b (3-F) | 0.821 | 0.220 | 82 | 73 | 0.47 |
| 16c (4-Cl) | >10 | 0.136 | 71 | 34 | 0.97 |
| 16d (4-Br) | >10 | 0.427 | 42 | 0 | |
| 16e (4-I) | N.A.^{e} | 2.038 | N.A.^{e} | N.A.^{e} | |
| 16f (4-COCH₃) | 1.056 | 0.758 | 30 | N.A. | |
| 16g (4-CF₃) | 0.898 | 0.071 | 80 | 100 | 0.29 |
| 16h (3-CF₃) | 0.512 | 0.205 | 67 | 54 | 0.73 |
| 16i (4-CN) | 1.499 | 0.045 | 90 | 100 | 0.32 |
| 16j (4-NO₂) | 2.225 | 0.036 | 20 | N.A. | |
| 16k (4-OCH₃) | N.A. | No | 0 | 0 | |
| | | effect | | | |
| 16l (4-CH₃) | 1.552 | 8.087 | N.A. | N.A. | |
| 16m (4-phenyl) | >10 | 1.152 | 0 | 0 | |
| 16n (3-phenyl) | 3.660 | 4.770 | 0 | 0 | |
| 16o [4-(4- | 0.612 | 0.969 | 72 | 0 | 0.86 |
| fluorophenyl)] | | | | | |
| 16p [3-(4- | >10 | 1.069 | 54 | 81 | 0.99 |
| fluorophenyl)] | | | | | |
| 16q (4-ethynyl) | N.A. | 0.276 | 7.28^{f} | N.A. | |
| 16r [4-(4-OH- | >10 | No | 51.80^{f} | N.A. | 0.97 |
| but-1-yn-1-yl)] | | effect | | | |
| 16s (4- | N.A. | No | N.A. | N.A. | |
| carbamoyl) | | effect | | | |
| 16t (4-NH₂) | 0.223 | Agonist | — | N.A. | |
| 16u (4- | 1.382 | 1.153 | 20 | 0 | |
| NHCOOtBu) | | | | | |
| 16v (4- | >10 | No | — | N.A. | |
| NHCOCH₃) | | effect | | | |
| 16w (4- | >10 | No | N.A. | N.A. | |
| NHCOCH₂Cl) | | effect | | | |
| 16x (4- | >10 | 0.827 | N.A. | N.A. | |
| NHCOOCH₃) | | | | | |
| 3 (R- | 0.509 | 0.248 | — | — | |
| bicalutamide)^{g} | | | | | |
| 4 | 3.641 | 0.216 | — | — | |
| (enzalutamide)^{g} | | | | | |
| 5 (apalutamide)^{g} | 1.452 | 0.160 | | — | |
| 6 (darolutamide) | 0.011^{b} | 0.65^{b} | N.A. | N.A. | |

^{a}AR binding was determined by competitive binding of 1 nM [3H] MIB to recombinant
LBD of wildtype AR (wtAR). DHT was used in each experiment as a standard agent and the
values are normalized to DHT, with the IC₅₀ of DHT taken as 1 nM.
^{b}Inhibition of transactivation was determined by transfecting HEK-293 cells with full-
length wtAR, GRE-LUC, and CMV-renilla luciferase for transfection control. Cells were
treated 24 h after transfection with a dose response of compounds (1 pM to 10 μM) in the
presence of 0.1 nM R1881 (antagonist mode) or in the absence of R1881 (agonist mode).
Luciferase assay was performed 24 h after treatment using a dual luciferase (firefly and
Renilla) assay kit (Promega, Madison, WI).
^{c}SARD activity was assayed by treating LNCaP or 22RV1 cells for determining FL AR
(at 1 μM of antagonist) or SV AR (at 10 μM of antagonist) protein levels, respectively. Cells
were maintained in charcoal-stripped serum-containing medium for 48 hand treated with the
indicated doses of antagonist for 24 h in the presence of 0.1 nM R1881 (agonist). Cells were
harvested and Western blot for AR was performed using AR-N20 or PG-21 antibody that is
directed toward the NTD of AR and actin (internal control for protein loading). The AR FL
and AR SV bands were quantified and normalized to actin bands and represented as percent
inhibition from vehicle treated cells.
^{d}Result was reported in the literature in the same assay as described here.
^{e}N.A. means data not available.
^{f}The two values indicate SARD assays run with 1 and 10 μM of antagonist.
^{g}Transcriptional activation was performed in the same assay in antagonist mode and the
IC₅₀ values are reported.
^{h}Binding affinity and wtAR inhibition of transactivation were reported in the literature
for the mixture of diastereomers of darolutamide.

### Example 4

### Modifications of Aromatic A-Ring (Series II)

Compounds 21a-21j were prepared by the route shown in Scheme 2. Treatment of acid 11 with SOCh provided the acid chloride (R)-3-bromo-2-hydroxy-2-methylpropanoyl chloride (not shown), which was reacted with various amines (17) under basic Et3N conditions to furnish bromoamides 18 with different A-rings. Basic conditions (e.g., K2CO3) transformed bromoamides 18 to the oxirane intermediates 19, followed by coupling with various pyrazoles 20 under the sodium hydride basic conditions to produce the target compounds 21a-21j. The compounds were tested in vitro for AR activity (Table 3).

For the 4-F pyrazole, replacing a carbon (CH) with a nitrogen (N) at the 3′-position of the A-ring, that is, 3′-pyridino derivative of 10, delivered a more potent compound (21a) with AR inhibitory IC₅₀ value of 0.062 μM compared to its counterpart 10 (IC₅₀=0.199 M). However, in other instances, 3′-pyridino derivatives were equipotent or less potent than their phenyl A-ring analogues. The 3′-pyridino 21c (4-CN; 0.059 μM) showed almost equally potent AR inhibitory activity compared to its A-ring phenyl analog 16i (IC₅₀=0.045 μM). However, 3′-pyridino compounds 21b (4-CF₃) and 21d (4-NHCOOtBu) showed lower activity (IC₅₀ values of 0.208 μM and 6.108 μM, respectively) than their phenyl A-ring counterparts 16g and 16u. Other A-ring modifications of 10 decreased the AR inhibitory activity and % degradation when compared to 10 (0.199 μM; 100%/100%), including replacing the 3′-CF3 with a 3′-Cl (21e; 0.427 μM; 42%/0% degradation), replacing the 4′-CN with a 4′-NO₂ (21f; partial agonist; N.A. % degradation), and other modifications as in 21g-21k. Unlike other pyrazole propanamides, which show low or no AR LBD binding affinity (Kᵢ), it was found that the combination of 4-CN substituent in pyrazole and 3′-pyridino A-ring promoted the tight LBD binding seen for 21c (Kᵢ=0.089 μM) but relatively poor SARD activity (15%/N.A.).

TABLE 3
In Vitro AR Activity of 21a-21j (Series II)

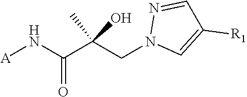

| | | Binding (Kᵢ)/ | | | | |
|---|---|---|---|---|---|---|
| | | Transactivation | | SARD Activity | | |
| | | (IC₅₀) | | (% degradation) | | |
| | | (μM) | | Full | Splice | |
| | Structure of the | Kᵢ | | Length^{a} | Variant^{a} | F.L. |
| | A-ring | (DHT = 1 | | (LNCaP) | (22RV1) | DC₅₀ |
| ID (R₁) | (A) | nM)^{a} | IC₅₀^{a} | at 1 μM | at 10 μM | (μM) |
| 21a (4-F) | 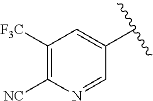 [SMILES: *c1cnc(C#N)c(C(F)(F)F)c1] | >10 | 0.062 | 54 | 81 | 0.88 |
| 21b (4-CF₃) | 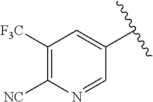 [SMILES: *c1cnc(C#N)c(C(F)(F)F)c1] | 2.286 | 0.208 | 10 | N.A.^{b} | — |
| 21c (4-CN) | 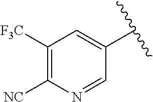 [SMILES: *c1cnc(C#N)c(C(F)(F)F)c1] | 0.089 | 0.059 | 10 | N.A.^{b} | — |
| 21d (4- NHCOOtBu) | 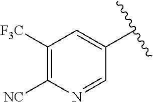 [SMILES: *c1cnc(C#N)c(C(F)(F)F)c1] | >10 | 6.108 | — | N.A.^{b} | — |
| 21e (4-F) | 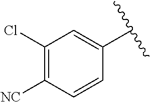 [SMILES: *c1ccc(C#N)c(Cl)c1] | >10 | 0.427 | 42 | 0 | — |
| 21f (4-F) | 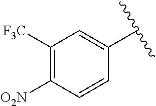 [SMILES: *c1ccc([N+](=O)[O-])c(C(F)(F)F)c1] | N.A.^{b} | Partial Agonist | N.A.^{b} | N.A.^{b} | — |
| 21g (4-F) | 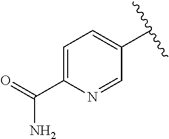 [SMILES: *c1ccc(C(N)=O)nc1] | >10 | No effect | 0 | N.A.^{b} | — |
| 21h (4-F) | 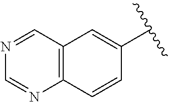 [SMILES: *c1ccc2ncncc2c1] | >10 | No effect | N.A. | N.A. | — |

^{a}AR binding, transactivation, and degradation assays were performed and values
reported as described in Table 2.
^{b}N.A. means data not available.

### Example 5: Disubstitution of the Pyrazole B-Ring (Series III)

Compounds 26a-26h were synthesized utilizing similar synthetic methods as in Schemes 1 and 2, as depicted in Scheme 3 and tested for AR activity (Table 4).

Compound 26a possessed two electron withdrawing groups (3-F and 4-Br) on the pyrazole ring and exhibited potent inhibitory activity (IC₅₀ value of 0.084 μM) and moderate to high efficacy AR FL and AR SV degradation (70-80% degradation). Compound 26a improved the AR inhibitory potency by 3-4 folds over the 3-F (16b; 0.220 μM; 82%/73%) and 4-Br (16d; 0.427 μM; 42%/0%) monosubstituted analogues and retained or improved upon degradation properties, supportive of further exploration of disubstitution. Replacing a carbon (CH) with a nitrogen (N) in the A-ring of 26a delivered the 3′-pyridino 26f, which was a very potent AR inhibitor with an IC₅₀ value of 0.035 μM but poor SARD activity (8%, 15%/N.A. for FL/SV) (Table 4). Compounds 26b-26e with di-substituents on pyrazole showed inhibitory activity comparable to 10 (0.199 μM) with AR inhibitory IC₅₀ values in the order of 26e (3-Br, 4-Cl; 0.138 μM)>26c (3-Br, 4-CN; 0.202 μM)>26b (3-Br, 4-(4-fluorophenyl); 0.285 μM)>26d (3-Cl, 4-methyl); 0.332 μM).

Compounds 26e (0.138 μM) and 26c (0.202 μM) did not improve upon their monosubstituted analogs 16c (4-Cl; 0.136 μM) and 16i (4-CN; 0.045 μM). Addition of halogens to 4-EDG pyrazoles at least partially rescued the activity, for example, compare to 26b (3-Br, 4-(4-fluorophenyl); 0.285 μM) and 160 (4-(4-fluorophenyl); 0.969 μM) and 26d (3-Cl, 4-methyl); 0.332 μM) and 16l (4-methyl; 8.087 μM). Again, these results suggest that the EWG strength of the pyrazole substituents contribute favorably to inhibitory activity. The 3′-pyridino A-ring version of 26c afforded a >10-fold less potent inhibitor 26g with an AR inhibitory IC₅₀ value of 5.481 μM despite 80% SARD efficacy in AR FL (but no efficacy in AR SV). Introducing an extra bromo (26g) or a phenyl (26h) group on the 3-position of the pyrazole greatly decreased the inhibitory activity to 5.481 or 0.579 μM, respectively, compared to 21c (4-CN; 0.059 μM). It was found that 3-Br and 4-CN substituents on pyrazole promoted a tighter LBD binding (Kᵢ=0.202 μM) for 26c and 3-F, 4-Br substituents on pyrazole (26a) delivered a potent inhibitor (IC₅₀=0.084 μM) while retaining the SARD activity in AR FL and AR SV (70%/80%).

TABLE 4
In Vitro AR Activity of 26a-26h (Series III)

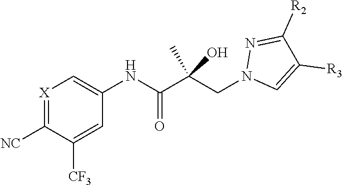

| | | | | Binding (Kᵢ)/ | | SARD Activity | | |
|---|---|---|---|---|---|---|---|---|
| | | | | Trans- | | (% degradation) | | |
| | | | | activation | | Full | Splice | |
| | | | | (IC₅₀) (μM) | | Length^{a} | Variant^{a} | |
| | | | | Kᵢ | | (LNCaP) | (22RV1) | F.L. |
| | | | | (DHT = | | at 1 μM, | at | DC₅₀ |
| ID | X | R₂ | R₃ | 1 nM)^{a} | IC₅₀^{a} | 10 μM | 10 μM | (μM) |
| 26a | CH | F | Br | 0.607 | 0.084 | 70 | 80 | 0.86 |
| 26b | CH | F | 4-F- | 0.601 | 0.285 | N.A.^{c} | toxic | |
| | | | phenyl | | | | | |
| 26c | CH | Br | CN | 0.202 | 0.181 | 41.23^{b} | 32 | |
| 26d | CH | Cl | CH₃ | 1.345 | 0.332 | 41.83^{b} | N.A.^{c} | |
| 26e | CH | Br | Cl | 4.935 | 0.138 | N.A.^{c} | N.A.^{c} | |
| 26f | N | F | Br | 0.567 | 0.035 | 8.15^{b} | N.A.^{c} | |
| 26g | N | Br | CN | N.A.^{c} | 5.481 | 40.80^{b} | N.A.^{c} | N.A. |
| 26h | N | phenyl | CN | N.A.^{c} | 0.579 | 9.55^{b} | N.A.^{c} | |

^{a}AR binding, transactivation, and degradation assays were performed and values
reported as described in Table 2.
^{b}The two values indicate SARD assays run with 1 and 10 μM of antagonist.
^{c}N.A. means data not available.

### Example 6: Modification of the Linkage Moiety (Series IV)

Compounds 29a-29f were synthesized utilizing similar synthetic methods as in Schemes 1-3, as depicted in Scheme 4 and 29a-29f were tested for their AR activity (Table 5 and Table 1).

Switching the chirality of 10 (the S-isomer) afforded the almost equipotent 29a (the R-isomer) with an AR inhibitory IC₅₀ value of 0.192 μM and slightly decreased to 84% degradation compared to 10 (100%). Removal of the 2-hydroxyl moiety from the 2-hydroxy-2-methyl propanamide linker of 10 produced 26b with reduced AR inhibitory activity (0.462 μM) and 60%/70% FL/SV SARD activity compared to 10 (0.199 μM; 100%/100%). Removal of 2-methyl and 2-hydroxy moieties from linkage of 10 to produce the linear propanamide 29c further decreased the AR inhibitory and SARD activities.

As an oxazolidin-2,4-dione linker variant of 10, compound 29d possessed groups similar to the amide and hydroxyl (as the oxygen in the carbamate) groups of the linker. 29d still showed activity but with substantially decreased AR inhibitory (1.131 μM) and SARD (18%, 50%/N.A.) activities compared to 10 (0.199 μM; 100%1100%). Acylation of the 2-hydroxy AR agonist 16t (4-NH₂) produced 29e which recovered some antagonist activity with AR inhibitory IC₅₀ value of 0.901 μM, whereas introducing a second amide into the linker and varying the pyrazole attachment position as in 29f produced an agonist. Although the linker element was not optimized in this initial SAR of Series IV, tolerance to chiral center inversion was again observed, and it was established that there is no absolute requirement for the 2-hydroxy-2-methylpropamide linker for inhibitory and SARD activities.

TABLE 5
In Vitro AR Activity of 29a-29f (Series IV)

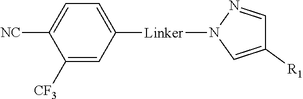

| | | | | SARD Activity | | |
|---|---|---|---|---|---|---|
| | | Binding (Kᵢ)/ | | (% degradation) | | |
| | | Transactivation | | Full | | |
| | | (IC₅₀) (μM) | | Length^{a} | Splice | |
| | | Kᵢ | | (LNCaP) | Variant^{a} | F.L. |
| | | (DHT = 1 | | at 1 μM, | (22RV1) | DC₅₀ |
| ID (R₁) | Linker | nM)^{a} | IC₅₀^{a} | 10 μM | at 10 μM | (μM) |
| 29a (4-F) (R-isomer) | 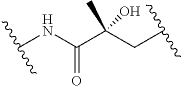 [SMILES: *C[C@@](C)(O)C(=O)N*] | >10 | 0.192 | 84 | N.A.^{b} | — |
| 29b (4-F) | 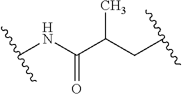 [SMILES: *CC(C)C(=O)N*] | >10 | 0.462 | 60 | 70 | 0.74 |
| 29c (4-F) | 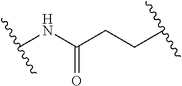 [SMILES: *CCC(=O)N*] | >10 | 2.124 | 35 | 40 | — |
| 29d (4-F) | 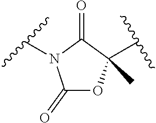 [SMILES: *N1C(=O)O[C@](*)(C)C1=O] | N.A.^{b} | 1.131 | 18.50^{c} | N.A.^{b} | — |
| 29e (4-NH₂) | 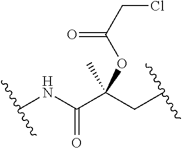 [SMILES: *C[C@](C)(OC(=O)CCl)C(=O)N*] | >10 | 0.901 | N.A. | N.A. | — |

^{a}AR binding, transactivation, and degradation assays were performed and values
reported as described in Table 2.
^{b}N.A. means data not available.
^{c}The two values indicate SARD assays run with 1 and 10 μM of antagonist.

The AR LBD affinity (for some compounds) and in vitro antagonist properties of Series 1-111 ranged from comparable to favorable relative to the known standard AR antagonists currently employed clinically for the treatment of PC. For example, 2, 4, 5, and 6 had LBD binding affinities of 0.509, 3.641, 1.452, and 0.011 μM (values for 3-5 are internally determined vs. for 6 is from the literature), and in vitro inhibition of 0.248, 0.216, 0.160, and 0.065 μM (values for 3-5 are internally determined vs. for 6 is from the literature); compared to 10 binding of >10 μM and antagonism of 0.199 μM. It was found that compounds 16b, 16c, 16g, 16h, 16i, and 16m from Series I, 21a from Series II, 26a and 26c from Series Ill, and 29a from Series IV exhibited relatively potent AR inhibitory IC₅₀ values in the range from 0.041 to 0.220 μM but, unlike 2 and 4-6, were SARDs with degradation activity values in the range from 100% to 45%. These compounds with the exception of 16m were comparable to improved inhibitors relative to known LED-targeted antiandrogens but possessed novel pan-antagonism and SARD activities.

### Example 7: In Vitro Metabolic Stability in Mouse, Rat, and Human Liver Microsomes

Compounds with potent inhibitory activity of each series were selected to be further evaluated for in vitro metabolic stability in mouse liver microsomes (MLM) with co-factors for enzymes of both phase I and phase II metabolism. The half-life (T_{1/2}) and intrinsic clearance (CLᵢₙₜ) values were calculated as a predictor of the distribution, metabolism and pharmacokinetic (DMPK) properties of these compounds (Table 6). The CLᵢₙₜ of these compounds was slower than previous generations of SARDs, producing relatively stable T_{1/2} values that range from 48.45 min to >360 min for these pyrazol-1-yl-propanamides (16b, 16g, 16h, 16i, 16m, 21a, 26a, and 29a) with six of the nine tested pyrazoles being stable for >360 min in MLM. This is a vast improvement when compared to previous SARD templates such as 1.15 min for tertiary amine 8, 12.11 min for indole 9 (Ponnusamy, et al. Cancer Res. 2017, 77, 6282-6298) and 9-36 min for a variety of indole and indoline B-ring compounds previously published in the same in vitro assay ((Dellis, et al. Expet. Opin. Invest. Drugs 2018, 27, 553-559) and an improvement over 10 (T_{1/2} of 77.96 min).

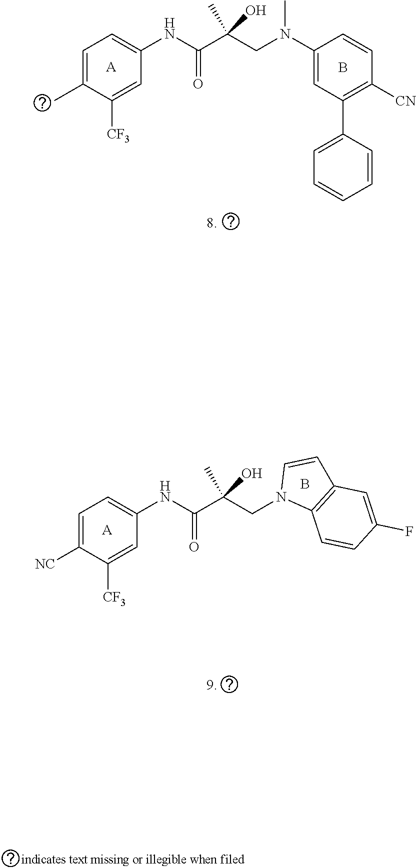

TABLE 6
In Vitro Metabolic Stability for Selected
Compounds in Mouse Liver Microsomes (MLM)

(DMPK) MLM^{a}

| Compound ID | T_{1/2} (min) | CLᵢₙₜ (mL/min/mg) |
|---|---|---|
| 10 (4-F)^{b} | 77.96 | 0.89 |
| 16b (3-F) | 64.07 | 1.02 |
| 16g (4-CF₃) | >360 | 0 |
| 16h (3-CF₃) | 330 | 0 |
| 16i (4-CN) | >360 | 0 |
| 16m (4-phenyl) | 48.45 | 14.31 |
| 21g (4-F) | >360 | 0 |
| 26a (3-F, 4-Br) | >360 | 0 |
| 29b (4-F) | >360 | 0 |
| 8^{b}/9^{b} | 1.15/12.11 | 208.8/57.26 |
| 4 (Enzalutamide) | 10.04 h ^{c} | 86.3^{d} |

^{a}compounds were incubated together with mouse liver microsomes (MLM) with co-factors for phases I and II provided, as described in the Experimental Section.
^{b}Reported previously in using the same method as in the Experimental Section.
^{c} T_{1/2} (h) after oral administration in humans as previously reported in ref.⁵⁸
^{d}CL (mL/h/kg) after oral administration in humans as previously reported.⁵⁸

The likely metabolic liability in aryl bicycles such as indoles and indolines may be aryl hydroxylation of the B-ring. The A ring and propanamide portions have been incorporated into many bioavailable compounds such as 2 (N-[4-cyano-3-(trifluoromethyl)phenyl]-3-(4-fluorophenyl)sulfonyl-2-hydroxy-2-methylpropanamide) and enobosarm ((2S)-3-(4-cyanophenoxy)-N-[4-cyano-3-(trifluoromethyl)phenyl]-2-hydroxy-2-methylpropanamide), leaving the B-ring as the likely metabolically labile site. A possible rationale for improved PK properties with pyrazoles is the elimination of some of the possible aryl hydroxylation sites on the B-ring. It is also possible that the increased positive charged on the 2-position nitrogen atom of the pyrazole makes the compounds poor substrates for metabolic enzymes and/or improves biological partitioning.

Four further compounds were also characterized in rat liver microsomes (RLM) and human liver microsomes (HLM) as these readouts are relevant to suggest the stability of compounds for in vivo testing in PD models such as the rat Hershberger assay and xenograft in SRG rats (see infra) and ultimately in the clinic (Table 7). Series I compounds 16c and 16g were stable in RLM (T_{1/2} of >120 min) but 16c was less stable in HLM (T_{1/2} of 102 min). 21a (3-pyridino, 4-F) and 26a (3-F, 4-Br) were stable (T_{1/2} of >120 min) in both RLM and HLM, which was similar to previously published data for 10 in RLM (181 min) and HLM (274 min) (Ponnusamy, et al. Clin. Cancer Res. 2019, 25, 6764-6780). The stability in RLM and HLM is consistent with the possibility of oral bioavailability of these pyrazoles, as previously seen with 10. However, 21a and 26a have improved in vitro efficacy relative to 16c and 10.

Correspondingly, if sufficiently high blood levels of 21a and 26a are attained and the compounds are distributed to the site of action, that is, the tumor(s) throughout the body, it may be possible to improve the efficacy to treat antiandrogen-resistant CRPC compared to 10. As a result, pyrazole compounds (16i, 21a, and 26a) with a variety of activity profiles were advanced to testing in the models of CRPC including resistance to 4 (MR49F cells harboring F876L AR point mutant) and 1 (LNCaP cells harboring the T877 A).

TABLE 7
In Vitro Metabolic Stability for Selective
Compounds in RLM and HLM

| | RLM^{a} | | HLM^{b} | |
|---|---|---|---|---|
| | | CLᵢₙₜ | | CLᵢₙₜ |
| | | (mL/ | | (mL/ |
| Compound ID | T_{1/2} (min) | min/mg) | T_{1/2} (min) | min/mg) |
| 16c (4-Cl) | >120 | 0 | 102 | 6.78 |
| 16g (4-CF₃) | >120 | 0 | >120 | 0 |
| 21a (4-F) | >120 | 0 | >120 | 0 |
| 26a (3-F, 4-Br) | >120 | 0 | >120 | 0 |

^{a}compounds were incubated together with RLM with cofactors for phases I and II provided, as described in the Experimental Section.
^{b}Compounds were incubated together with HLM with cofactors for phases I and II provided, as described in the Experimental Section.

### Example 8: In Vitro Pharmacodynamics in Models of Castration Resistant Prostate Cancer

Compounds were screened in vitro in a competitive LBD binding assay (Ki), inhibitory AR transactivation assay (IC₅₀), and AR FL (in LNCaP cells) and AR SV (in 22RV1 cells) degradation assays (% degradation) (Tables 2-5 above). Once strong in vitro screening profiles were accomplished for single molecule(s), in vitro metabolic stability criteria were also considered in the selection of the compounds to be tested further in vitro (Tables 6 and 7 above). In order to improve the efficacy in in vivo testing, compounds were sought with superior in vitro screening profiles compared to 10 and tested further for transactivation selectivity between AR and PR, AR target gene expression in LNCaP cells, and proliferation studies in Enz-R PC cells (MR49F LNCaP cells).

Mutant AR and wtPR Antagonist Effects

The selected compounds 16i (4-CN), 21a (3-pyridino, 4-F), 26a (3-F, 4-Br), and 10 (4-F) were tested for their ability to antagonize a LBD point mutant AR, which confers an enzalutamide (4) resistant (Enz-R) phenotype to PC cells. This F876L-mutant AR or wildtype PR (wtPR) were transfected into COS cells, a non-PC cell line, and quantified by luciferase assay (FIG. 1). Compounds 16i, 21a, 26a, and 10 robustly inhibited the F876L mutant AR with IC₅₀ values of 0.043, 0.063, 0.084, and 0.219 μM (FIG. 1) that are comparable to wtAR IC₅₀ values of 0.045, 0.062, 0.084, and 0.199 μM (Tables 2-4). The ability to equipotently inhibit F876L and wtAR indicates these SARDs exhibit pan-antagonism in a model of Enz-R. Further, this panantagonism cannot be explained by the AR LBD Ki values of 1.499, >10, 0.607, and >10 μM for 16i, 21a, 26a, and 10. Moreover, the increased potency of 16i, 21a, and 26a relative to 10 in wtAR inhibitory potency translated into this model of Enz-R.

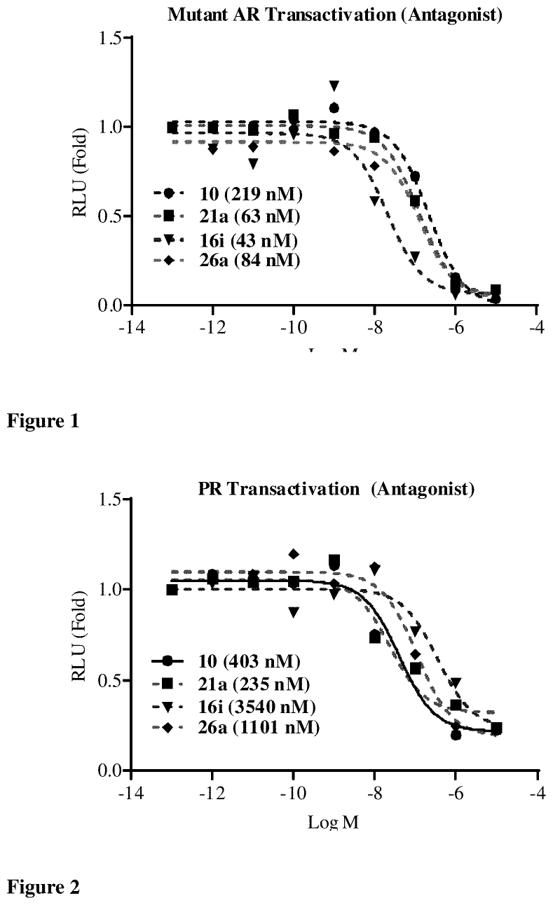

These molecules also inhibited wtPR activity with IC₅₀ values of 3.540, 0.235, 1.101, and 0.403 μM for 16i, 21a, 26a, and 10 (FIG. 2) vs. 0.045, 0.062, 0.084, and 0.199 μM for wtAR inhibition (Tables 2-5). Though wtPR inhibition was conserved, selectivity ratios ([PR IC₅₀]/[AR IC₅₀]) varied with values of 79-, 3.8-, 13.1-, and 2.0-fold for the compounds selected for testing, suggesting that AR selectivity could also be optimized with further testing. Importantly, none of these molecules had any effect on GR, MR, or ER transactivation (data not shown).

### AR-Target Gene Expression in CRPC Cells.

An AR target gene inhibitory experiment was performed to determine the effect of lead pyrazole 26a on R1881-induced AR target gene expression in LNCaP cells (FIG. 3). The LNCaP cell line is a very well characterized model of CRPC that expresses the T877 A point mutation of AR that confers resistance to 1. Compound 26a was chosen as the lead pyrazole as 26a possessed a balance of high potency inhibition (0.084 μM) and high efficacy degradation (70-80% for both AR FL and AR SV) with 3,4-disubstitution that blocked metabolism relative to 10 (T_{1/2}>360 min vs. 77.96 min in MLM (Table 6)) and 26a is also stable in RLM and HLM (>120 min). Consistent with the nM inhibition of wtAR (0.084 μM; Table 4) and F876L AR (0.084 μM; FIG. 1) transactivation, FKBP5 gene expression in LNCaP cells was robustly inhibited by 26a at concentrations as low as 0.1 μM, indicating that the antiandrogenic effects include inhibition of endogenous gene expression (FIG. 3) in another model of antiandrogen resistant CRPC without loss of potency. As expected, the antiandrogen 4 also inhibited expression of the FKBP5 but at a slightly lower potency. The same results were observed with other ARtarget genes such as PSA and TMPRSS2 (data not shown). Cumulatively, the above data above support that 26a has pan-antagonist effects in at least wtAR (Table 4), F876L (FIG. 1), T877 A (FIG. 3), and AR SV (Table 4).

### Proliferation Studies in Enzalutamide-Resistant LNCaP Cells.

Proliferation studies were conducted with 26a to confirm that potent inhibition of ARdependent gene expression in a model of CRPC harboring the T877 A antiandrogen resistance mutation (i.e., LNCaP cells) translated into antiproliferation in an even more refractory model of CRPC, that is, MR49F LNCaP cells harboring F876L and T877 A point mutations of AR. As mentioned above, the F876L mutation confers enzalutamide (4) resistance (Enz-R) to MR49F cells; however, MR49F cells remain dependent on the AR for growth. MR49F cells were tested in the presence of a titrated dose of 26a or 4 as shown in FIG. 4. Compound 26a demonstrated dose responsive antiproliferation that showed potent, but partial, efficacy (˜50-60% reduction from vehicle) at doses as low as 0.1 μM. The Enz-R of the MR49F model was demonstrated as the antiproliferation of 4 was ˜100-fold less potent. For example, 10 μM of 4 produced effects comparable to 0.1 μM of 26a, which was weak at ˜20% efficacy and not significant different from vehicle. Assuming that 26a can reach the tumors, this potent antiproliferation suggests that 26a may perform well in in vivo models of Enz-R CRPC.

### AR FL (F876L) and AR SV (AR-V7) Degradation in Models of CRPC

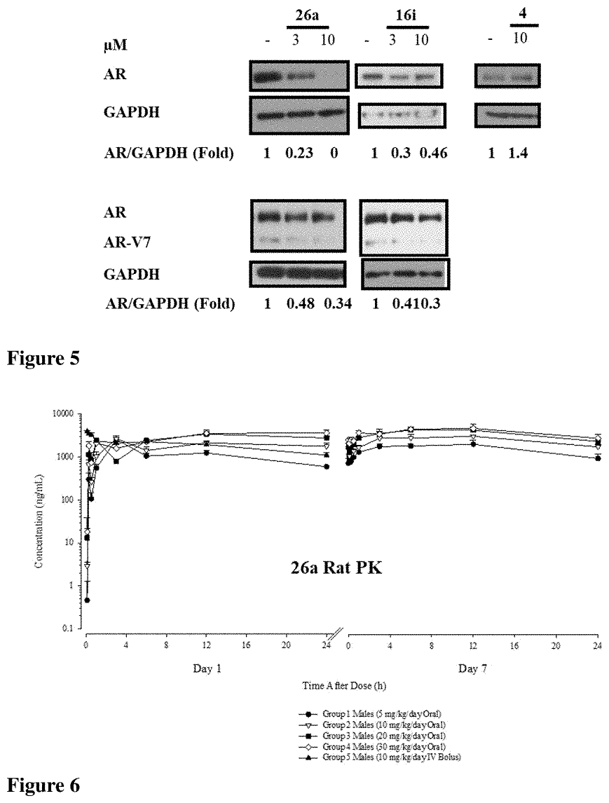

FL AR degradation studies in MR49F cells were performed in order to confirm that the robust in vitro AR antagonism profiles of 16i (0.045 μM, 90%, 100% in wtAR inhibition, AR FL and AR SV degradation assays) and 26a (0.084 μM, 70%, 80%) predicted SARD activity in this model of highly refractory CRPC. Compound 26a possessed the ability to suppress ARdependent gene expression in LNCaP cells and suppress proliferation in MR49F cells as described above and was able to also degrade the FL AR (FIG. 5 upper panel) in the Enz-R CRPC setting. Western blotting is not a quantitative method and it can be difficult to compare AR levels between compounds based on relative band densities. Accordingly, GAPDH was also included as a protein loading control in each lane. The levels of AR are normalized to the level of GAD PH in that lane. The western blots were quantified densitometrically and the AR/GAD PH values are represented as fold change (under blots in FIG. 5) or percent change from vehicle treated cells (Tables 2-5).

High efficacy SARD activity was observed with 26a at 3 μM and complete degradation at 10 μM (FIG. 5, top panel), indicating that this mutant AR FL that confers Enz-R in MR49F LNCaP cells is susceptible to destruction by 26a. 16i also demonstrated SARD activity but not full efficacy, whereas 4 produced no AR degradation in MR49F cells. The lower panel demonstrates that the SARD activity is not just present for T877 A (LNCaP; Tables 2-5) and F876L/T877 A (MR49F LNCaP cells; FIG. 5 upper panel) AR FL with point mutations in the LBD but also can degrade AR SVs such as the AR-V7 that lack the expression of the LBD (22RV1 cells; lower panel of FIG. 5). As shown in Tables 4 and 2 (see AR SV degradation column), 26a and 16i were able to reduce AR-V7 levels in 22RV1 cells at 10 μM. FIG. 5 confirmed AR-V7 SARD activity at 3 and 10 μM, but % degradation was not complete for either SARD in this particular experiment. Lower % degradation for AR SV than AR FL is consistent with earlier reports and Tables 2-5, which revealed that AR SV degradation can be complete but generally at higher treatment concentrations (screened at 10 M) than for AR FL (screening at 1 μM). PCs expressing AR SV s possess no binding site for traditional (or canonical) antiandrogens to bind AR, are associated with poor prognosis, and are believed to be pan-resistant to approved therapies including 1-7.66 Accordingly, these pyrazole SARDs and pan-antagonists, such as 10, 21a, and 26a that possess PK properties compatible with oral administration at low dose afford a very broad scope of AR antagonistic abilities in at least:
1) wtAR (IC₅₀ values in Tables 2-5),
2) T877 A (LNCaP AR FL degradation in Tables 2-5 and inhibition of AR-dependent gene expression in FIG. 3),
3) F876L (inhibition in COS cells in FIG. 1),
4) F876L/T877A co-mutant (proliferation in MR49F cells in FIG. 4),
5) AR-V7 (degradation of AR SV in 22RV1 cells in Tables 2-5 and FIG. 5), and
6) AR amplification/overexpression (see VCaP data reported infra).

The broad scope AR antagonism across various resistance-conferring AR mutants helps to ensure that treated tumors that are evolving to contain these and/or other AR mutations will remain sensitive to the SARDs and pan-antagonists as described herein. Further, these SARDs and pan-antagonists performed well in models of AR overexpression and/or AR gene recombination such as present in VCaP cells suggesting these PCs will not be able to resist this treatment either. In view of the fact that SARD activity may not be necessary for these activities, these compounds act as AR pan-antagonists. Compound 26a was tested as one of lead SARDs and pan-antagonists in vitro and subjected to a series of in vivo tests to describe its pharmacokinetic and pharmacodynamic profiles in healthy rats and models of antiandrogen resistant PC in rats.

### Example 9: In Vivo Rat Pharmacokinetics

Rat PK studies were conducted to confirm that pyrazole 26a possessed improved PK properties compared to previous generations of the SARDs. Optimized PK properties within the pyrazole template provide the best chance to reveal optimized in vivo PD profiles for the molecules with their unique AR mechanism of action in in vivo models of advanced PCs.

Male Sprague Dawley rats were given a single oral (po) daily dose on seven consecutive days or a single intravenous (iv) dose on day 1, and blood was sampled periodically at 0.083, 0.25, 0.5, 1, 3, 6, 12, and 24 h post dose. The doses of 5, 10, 20, and 30 mg/kg po (groups 1-4) and 10 mg/kg iv (group 5) were selected based on in vivo efficacies seen in a series of pilot experiments, which were similar to the Hershberger study discussed in detail in this section. Concentration-time curves were plotted from this data for 26a (FIG. 6), and the PK parameters were calculated for 26a from this data (Table 8).

TABLE 8
Summary of 26a pharmacokinetic parameters

| | Dose | | | | | | |
|---|---|---|---|---|---|---|---|
| | Level | C₀ | | DN Cₘₐₓ | | AUC₀₋₂₄ | DN AUC₀₋₂₄ |
| Dose | (mg/kg/ | (ng/ | Cₘₐₓ | (ng/mL)/ | Tₘₐₓ | (h*ng/ | (h*ng/mL)/ |
| Group | day) | mL) | (ng/mL) | (mg/kg/day) | (h) | mL) | (mg/kg/day) |
| 1 | 5 | NA | 2570 | 515 | 3.00 | 26,800 | 5350 |
| 2 | 10 | NA | 2680 | 268 | 3.00 | 44,600 | 4460 |
| 3 | 20 | NA | 3420 | 171 | 12.0 | 64,100 | 3200 |
| 4 | 30 | NA | 3650 | 122 | 24.0 | 71,500 | 2380 |
| 5 | 10 (iv) | 4200 | 3940 | 394 | 0.083 | 45,500 | 4550 |

C₀—Back-extrapolated concentration at time 0 (Group 5 only), Cₘₐₓ—Maximum observed concentration. DN Cₘₐₓ—Dose normalized Cₘₐₓ, calculated as Cₘₐₓ/dose level. Tₘₐₓ—Time of maximum observed concentration. AUC₀₋₂₄—Area under the concentration-time curve from time 0 to hour 24, estimated by linear trapezoidal rule. DN AUC₀₋₂₄—Dose normalized AUC₀₋₂₄, calculated as AUC₀₋₂₄/dose level.

Like 10, compound 26a demonstrated a robust PK profile in rats characterized by micromolar blood levels and a long terminal elimination half-life (t₁/2) (Table 8) consistent with daily oral dosing. An advantage of 26a over 10 is its relatively long t½, which is in excess of 24 versus 2.6 h (calculated based on the 7 day rat PK data reported in Ponnusamy's paper for 10) (Ponnusamy, et al. Clin. Cancer Res. 2019, 25, 6764-6780). The exact t½ value of 26a could not be calculated as the t½ was longer than the 24 h dosing interval (FIG. 6). 26a had decreasing oral bioavailability at higher doses as revealed by the decreasing dose-normalized area under the concentration-time curve from 0 to 24 h (DN AUC0-24) values and increasing time of maximum concentration (Tₘₐₓ) values for groups 1-4 with increasing 26a dose (Table 8). The calculated oral bioavailabilities for 5, 10, 20, and 30 mg/kg doses of 26a were 1.18, 0.982, 0.705, and 0.524. The longer t1/2 of 26a relative to 10 at least partially offset the decreasing oral bioavailability at high doses and 26a attained marginally increased absolute exposures compared to 10. For example, the AUC₀₋₂₄ values for 30 mg/kg po 26a and 10 were 71,500 and 62,000 h*ng/mL, respectively. The latter value, again, is calculated from the 7 day rat PK data presented in Ponnusamy's paper (Ponnusamy, et al. Clin Cancer Res. 2019, 25, 6764-6780).

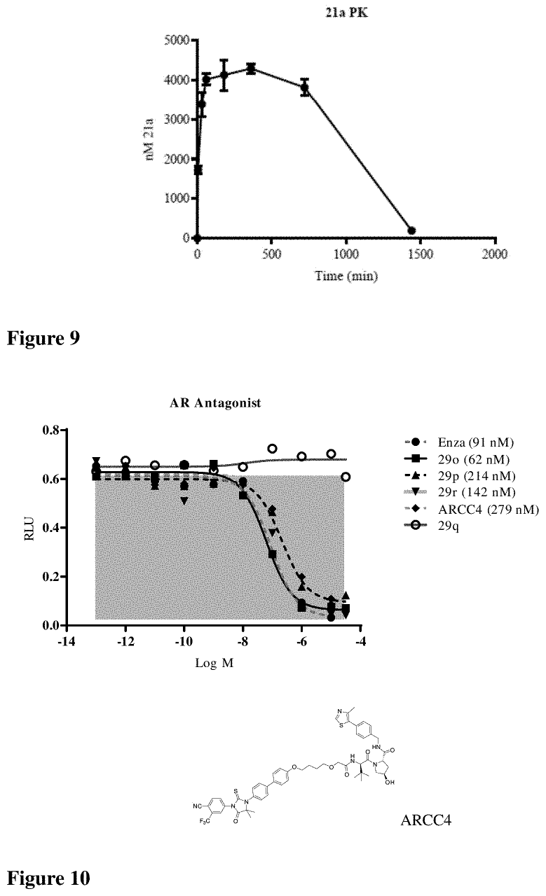

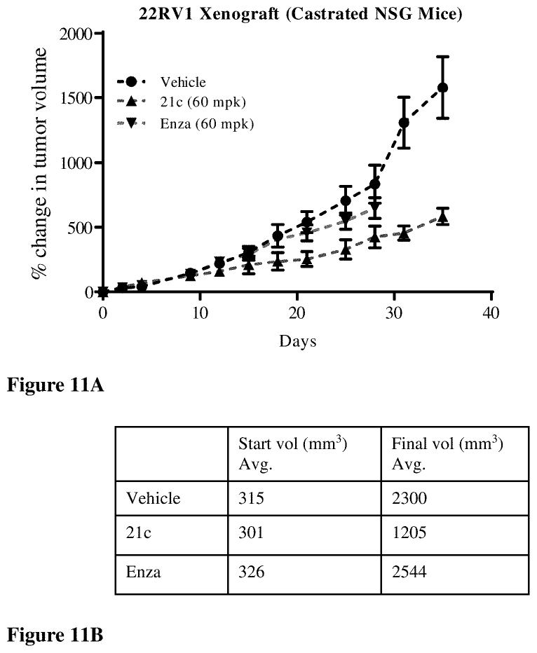

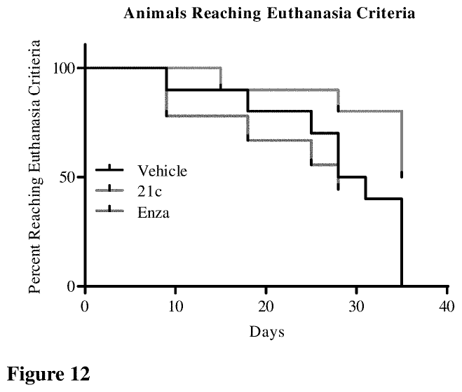

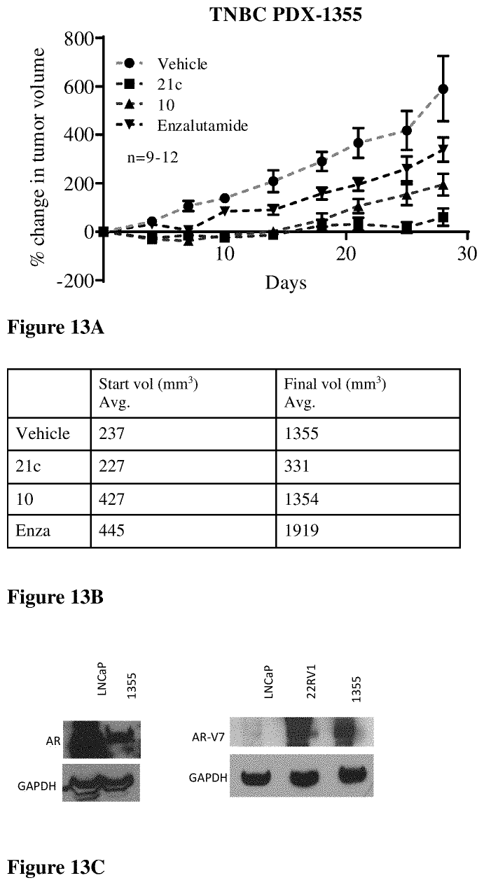

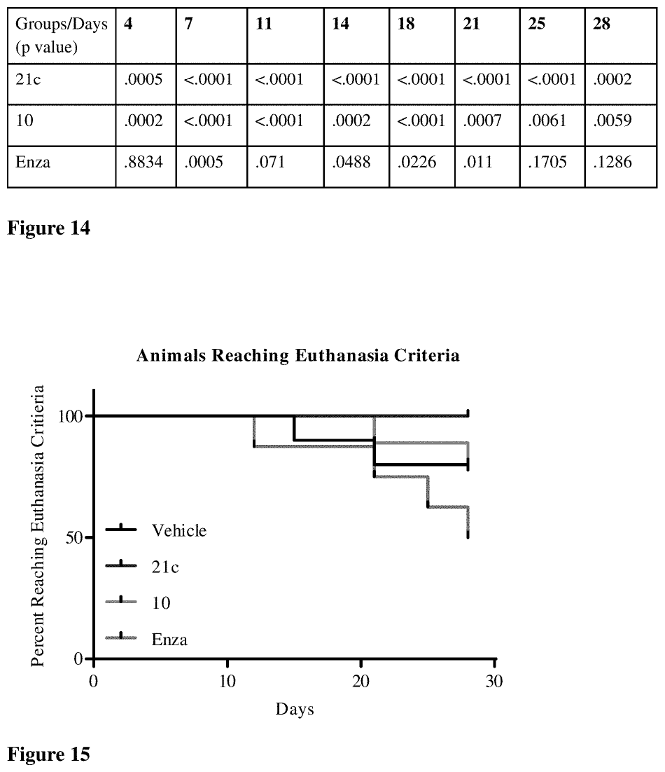

Compound 26a exhibited a PK profile sufficiently robust to maintain high blood levels in vivo via oral daily dosing in rats. Also shown is preliminary rat PK data for 30 mg po 21a (FIG. 9). The concentration versus time plot demonstrated reduced in vivo stability, with the vast majority of 21a eliminated by 24 h, which is in sharp contrast to 30 mg po 26a where blood levels at 24 h were barely reduced from their Cₘₐₓ (FIG. 6). Compound 21a at 30 mg po demonstrated sufficiently low CL to allow observation of its PD character in rats. Despite a potent in vitro panel of activities, 16i demonstrated lethality at 5 mg/kg in vivo. Compounds 21a and 26a were studied in rat Hershberger assays, and 26a was chosen as one of the leads for xenograft studies.

The micromolar Cₘₐₓ blood levels and long t½ observed for 26a suggested PK properties in rats consistent with revealing any high efficacy AR antagonism of 26a in vivo that was engendered by the data in Examples 2-8. These examples demonstrated that 26a inhibited a broad spectrum of antiandrogen activities in vitro with increased potency compared to 10, including in the models of antiandrogen-resistant CRPC. Oral daily dosing in rats with 26a should be able to maintain blood levels above the IC₅₀ value of AR antagonism (Table 4) and inhibitory effects on AR-dependent transcription (FIG. 3) and proliferation (FIG. 4), as would be necessary to suppress the AR axis in AR-dependent xenografts. Further, the low micromolar drug levels seen for 21a (FIG. 9), 26a (FIG. 6 and Table 8), and 102 were in excess of DC so values for 21a (880 nM), 26a (860 nM), and 10 (740 nM) (see Tables 2-4), suggesting that SARD activity may contribute to in vivo AR antagonism, as seen previously with 10 where intratumoral degradation was observed.

### Example 10: In Vivo Androgen Receptor Antagonist Activity

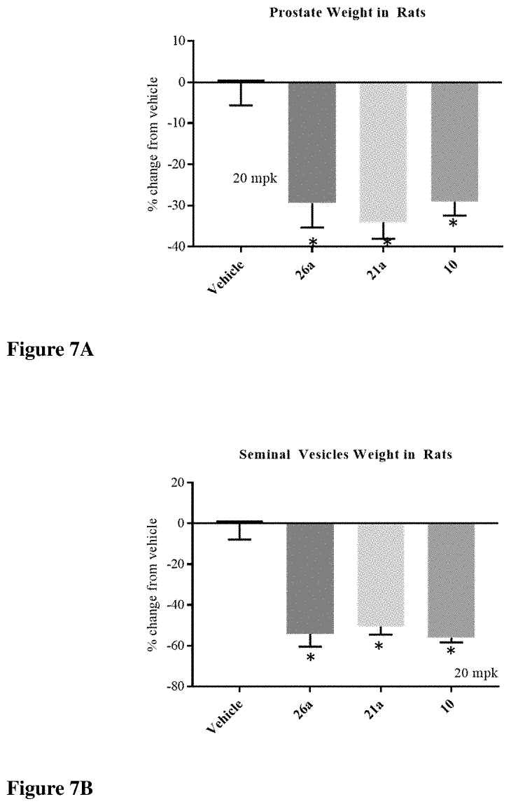

Hershberger Assays. In order to find whether these pyrazolylpropanamide compounds with robust PK properties have clinically meaningful SARD and pan-antagonist activity in vivo, Hershberger assays were performed in intact rats for 21a and 26a which have demonstrated oral bioavailability in rats (FIGS. 9 and 7). The Hershberger assay has been used to demonstrate anabolic selectivity of androgens for decades. Rat ventral prostate (VP), seminal vesicle (SV), and levator ani (LA) muscle are AR-dependent tissues whose size (reflected by their weight) responds rapidly to castration.

Upon castration, these organs atrophy within 3-7 days to organ weights that are approximately 85% (VP), 90% (SV), and 50% (LA) reduced compared to their intact organ weights. Traditionally, agonists are dosed to prevent (whereby agonist is given upon castration) or restore (agonist is given after tissue atrophies) anabolic tissue weights [LA or other skeletal muscles and bone (the latter takes months not days to atrophy and restore)] to intact levels or greater, without increasing androgenic tissue (SV or VP) weights back to intact levels. As employed herein, that is, antagonist mode, young intact animals were used wherein the endogenous androgen milieu provided AR-mediated support for the VP, SV, and LA weights, as reflected by the 0% change for the vehicle columns in FIGS. 7A and 7B.

Exogenous antagonists 21a and 26a, with potent in vitro inhibition [0.062 and 0.084 μM (Tables 3 and 4)], were dosed to observe their AR antagonism in vivo. Improved potency of in vivo AR antagonism was seen for derivatives of 10 with (1) the addition of the 3′-pyridino N to 10 as in 21a or (2) an additional halogen on the pyrazole such as 3-F or 4-Br in 26a. At 20 mg/kg of 21a and 26a, that is, one-third of the dose of 10 mentioned above, VP weights were reduced by approximately 35 and 30% (FIG. 7A), demonstrating that the in vivo PD properties intrinsic to 21a and 26a are observable at lower doses relative to 10. In SV at this dose, approximately 45-50% reductions were provided by 21a and 26a. Consistent with Example 9, these results confirm that orally administered 26a was absorbed and distributed to the site of action in AR target organs and suggest that these compounds should also distribute to tumors in xenograft models and exert antitumor effects in sensitive models.

Enz-R (MDVR) VCaP Xenografts in Rats. The VCaP cell line is derived from a vertebral bone metastasis from a patient with hormone refractory PC (https://atcc.org/Products/all/CRL-2876.aspx; accessed Jan. 20, 2020). 55 VCaP is commonly used as a model of CRPC, which expresses both AR SV (AR-V7) and overexpression of AR FL (TMPRSS2-ERG gene fusion). VCaP, the parental cell line for the MDVR VCaP used in the experiments below, is a model of highly advanced PC where multiple mechanisms of hormone resistance have emerged in response to androgen ablation in a single AR axis-driven cell line. The parental VCaP cells are nonetheless sensitive to enzalutamide (4); however, MDVR VCaP cells possess acquired Enz-R in addition to the resistance mechanisms in the parental cell line. Previously, it was observed that VCaP are partially sensitive to 4, whereas MDVR VCaP are not sensitive (Ponnusamy, et al. Clin. Cancer Res. 2019, 25, 6764-6780).

Following the demonstration with 26a of an in vitro screening panel that was superior to 10, in vitro activity in MR49F (an Enz-R LNCaP cell line) and in vivo antagonism in Hershberger assays, the activity of 26a was shown in Enz-R MDVR VCaP xenografts. To allow a direct comparison of 26a with 10, the MDVR VCaP xenografts were performed as published for 10 (Ponnusamy, et al. Clin. Cancer Res. 2019, 25, 6764-6780). For 10, castration was not necessary to show the efficacy in this model (unlike all previous AR antagonists to our knowledge); however, 10 was not stable in mice; therefore, intact SRG rats were used as the host for MDVR VCaP xenograft experiments.

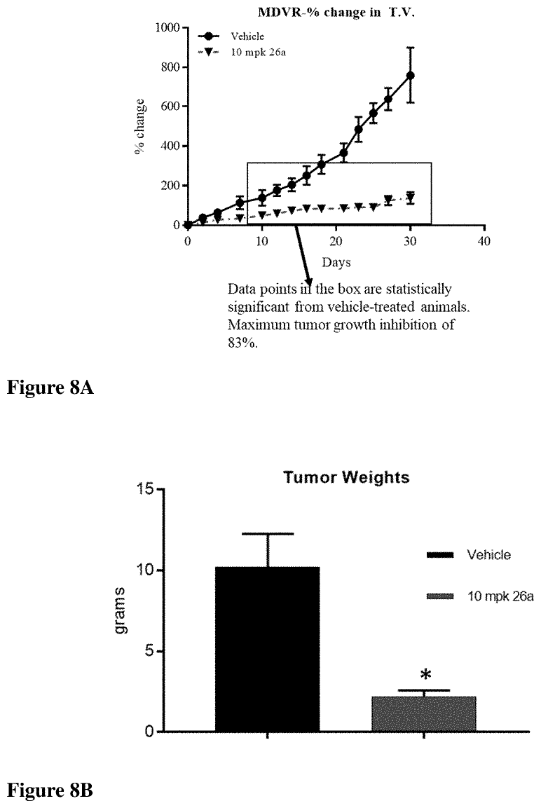

Treatment of intact SRG rats (studies performed at HERA Biolabs, Lexington Ky.) with 10 mg/kg po daily of 26a produced comparable efficacy of up to 83% TGI (FIG. 8A) versus 10 required 20-30 mg/kg to achieve similar results whereas 4 failed to durably achieve any effect (not shown; previously published) (FIG. 5) (Ponnusamy, et al. Clin. Cancer Res. 2019, 25, 6764-6780). Tumor weights measured at the end of the study also demonstrated a significant inhibition (FIG. 8B). Consistent with the exhibited high-potency antitumor activity, 26a was observed in this study at an average concentration within the tumors of 881 nM, which is 10-fold higher than its IC50 value in wtAR or F876L (both 84 nM). Further, intratumoral levels were only slightly reduced from the 1319 nM average concentration of 26a in the blood of these animals (Table 9). This supports efficient distribution of 26a into tumors, in addition to VP and SV, and supports its use in advanced PC.

TABLE 9
Serum and Tumor Drug Concentration of 26a

26a (3-F, 4-Br)

| | Serum (nM)a | Tumor (nM)a |
|---|---|---|
| animal 1 | 1611.663 | 962.6859 |
| Animal 2 | 1360.036 | 913.4912 |
| Animal 3 | 1143.556 | 666.0278 |
| Animal 4 | 1160.002 | 983.6887 |
| average | 1318.814 | 881.4484 |
| S.E. | 109.3146 | 73.29447 |

aTwenty to 24 h after the last dose (day 28), the animals were sacrificed, and blood and tumors were collected for further analysis. The serum was separated from blood, and drug concentration in serum and tumor was measured using the LC-MS/MS method (n = 4).

The in vitro DC₅₀ values (concentration of the 50th percentile of degradation efficacy) in LNCaP cells for 21a (880 nM) and 26a (860 nM) reported in Tables 3 and 4 were comparable to the intratumoral levels attained in the MDVR VCaP xenografts. Despite the different cell types between in vitro and in vivo studies, the data suggest the possibility of suboptimal exposures for full-efficacy SARD activity in the tumors of this experiment. This presumed half-efficacy intratumoral SARD activity may contribute to the TGI. It may be possible to improve the antitumor activity with increased intratumoral levels, that is, at increased dose of 26a, or with improved degradation potency analogues.

The results clearly indicate that 26a was stable in rats and was very potent and highly efficacious in this AR overexpressing and AR-V7 expressing model of Enz-R CRPC. The results further suggest that the improved PK and PD of 26a translated into more potent in vivo efficacies compared to 10, providing a dose-sparing SARD and pan-antagonist if, as of yet to be unobserved, toxicities become dose limiting. Further, the improved PK may translate into improved penetration throughout the cancer patient allowing better suppression of distant metastatic growth. All the above increase the chances of observing clinically significant reduction in disease burden when trialed in a human population (HLM studies in Table 7) expressing a broad spectrum of CRPC-resistant mechanisms. This population would still be sensitive even if expressing AR SVs (like AR-V7), AR gene amplications to overexpress AR (like TMPRSS2-ERG), or LED-directed antiandrogen resistance (like Enz-R and/or darolutamide resistance observed in MR49F or MDV VCaP cells) or combinations thereof as in MDVR VCaP.

These results confirm that for 26a, the in vitro screening paradigm was successful in identifying an improved lead compound from SARDs and pan-antagonists that was highly efficacious in an in vivo model of CRPC. Though full-efficacy in vitro SARD activity such as published for 10 is unique and should be beneficial in AR-dependent disease, it may not be necessary for efficacy in the clinic. This is supported by the more potent and comparable efficacy antitumor activity in vivo for 26a, which was not a full-efficacy SARD in vitro (70%/80%; Table 4), unlike 10 (100%/100%; Table 2). Although exact and incontrovertible mechanistic explanations of the high efficacy of 26a are not possible, its potent in vivo efficacy is also incontrovertible. The pyrazole template represents the optimal B-ring template presented to date, and 26a is one of optimized leads from this template. 10 or 26a is believed to hold great potential for overcoming multiple mechanisms of CPRC present in the clinic.

Compounds 16c, 16g, 16i, and 16j from Series I; 21a and 21c from Series II; and 26a, 26c, 26e, and 26f from Series III exhibited potent inhibitory activity in vitro, while compounds 16b, 16c, 16g, and 16i from Series I; 21a from Series II; and 26a and 26g from Series III possessed potent SARD activity in vitro (Tables 2-4). Compared to previous SARD templates such as 1.15 min for the tertiary amine 8 and 12.11 min for lead indole 9 (Ponnusamy, et al. Cancer Res. 2017, 77, 6282-6298), these pyrazolylpropanamide compounds, such as 16g, 16i, 21a, and 26a, significantly improved their stability in vitro in MLM (Table 6), and 21a and 26a were stable in RLM and HLM (Table 7). Compounds 16i, 21a, and 26a robustly inhibited the F876L-mutant AR with IC50 values of 0.043, 0.063, and 0.084 μM (FIG. 1), as well as inhibited wt PR activity with IC50 values of 3.540, 0.235, and 1.101 μM (FIG. 2). Compound 26a effectively inhibited the expression of FKBP5 in LNCaP cells at concentrations as low as 0.1 μM, indicating that the antiandrogenic effects include inhibition of endogenous gene expression (FIG. 3), as well as demonstrated dose-responsive antiproliferation at doses as low as 0.1 μM (FIG. 4). Compound 26a also produced superior in vivo rat PK and PD properties compared to 10 and 21a, with relatively long t_{1/2} values that were well in excess of 24 h (FIG. 6) and AR antagonism in rat Hershberger assay, with approximately 30% (VP), and 50% (SV) reduced compared to their intact organ weights (FIG. 7), which was comparable to 21a.

Enz-R (MDVR) VCaP xenograft experiments with 10 mg/kg po daily of 26a in an intact rat model demonstrated high drug levels intratumorally (881 nM) and produced an efficacy of 83% TGI (FIG. 8A), which was comparable to 10 at 20-30 mg/kg po. The results clearly indicate that 26a was very potent and highly efficacious in this AR overexpressing and AR-V7 expressing model of Enz-R CRPC and collectively satisfied all the criteria for a next-generation AR antagonist for Enz-R prostate cancer.

The pyrazolylpropanamide compounds as described herein are selective androgen receptor (AR) degraders (SARDs) and pan-antagonists that exert broad scope AR antagonism. Pharmacological evaluation demonstrated that these small molecules exhibited unique SARD and pan-antagonist activities. These compounds exhibited potent and broad spectrum AR antagonist activities including potent in vivo activities and promising distribution, metabolism, and pharmacokinetic (DMPK) properties.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating prostate cancer in a subject in need thereof, comprising administering to the subject a therapeutically effective amount of a compound represented by the structure of formula I 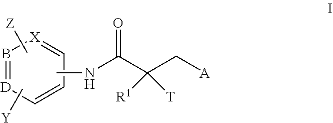
wherein
T is OH;
R¹ is CH₃;
Y is H, CF₃, F, I, Br, Cl, or CN;
Z is H, NO₂, CN, halogen, COOR, COR, NHCOR, or CONHR;
or Y and Z form a 5 to 8 membered fused ring;
X and Dare each CH or N;
B is a bond or CH, and when B is a bond, D=B-X is represented by D-X;
R is H, alkyl, haloalkyl, alkyl-OH, aryl, F, Cl, Br, I, or OH;
A is a five-membered unsaturated heteroaryl having at least one nitrogen atom, optionally substituted with at least one of Q¹, Q², Q³ and Q⁴, each independently selected from linear or branched alkyl, haloalkyl, CF₃, aryl, F, Cl, Br, I, CN, NO₂, OR, benzyl, alkynyl, SO₂N(R)₂, NH COOR, N(R)₂, NHCOR, CONHR, COOR, or COR;
wherein said alkyl, alkynyl, and aryl are each optionally substituted with halogen, CN, or OH,
or its optical isomer, pharmaceutically acceptable salt, hydrate or any combination thereof.

2. The method according to claim 1, wherein said compound is represented by a compound of formula IIA or formula IIB:

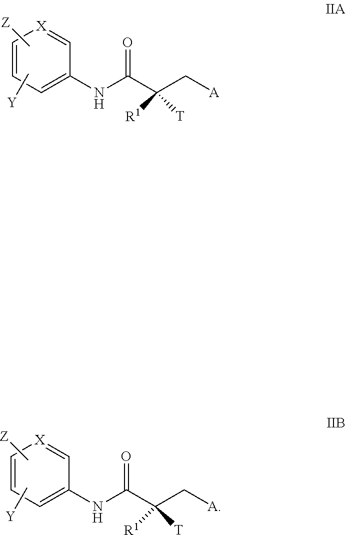

3. The method according to claim 1, wherein said compound is represented by a compound of formula III: 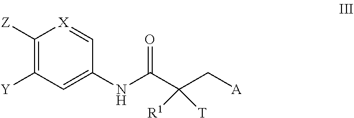
wherein
T is OH;
R¹ is CH₃;
Y is H, CF₃, F, I, Br, Cl, or CN;
Z is H, NO₂, CN, halogen, COOR, COR, NHCOR, or CONHR;
or Y and Z form a 5 to 8 membered fused ring;
X is CH or N;
R is H, alkyl, haloalkyl, alkyl-OH, CF₃, CH₂Cl, CH₂CH₂Cl, aryl, F, Cl, Br, I, or OH;
A is a pyrrole, pyrazole, triazole, or imidazole, each optionally substituted with at least one of Q¹, Q², Q³ and Q⁴, each independently selected from linear or branched alkyl, haloalkyl, CF₃, aryl, F, Cl, Br, I, CN, NO₂, OR, benzyl, alkynyl, SO₂N(R)₂, NHCOOR, N(R)₂, NHCOR, CONHR, COOR, or COR; wherein said alkyl, alkynyl, and aryl are each optionally substituted with halogen, CN, or OH,
or its optical isomer, pharmaceutically acceptable salt, hydrate or any combination thereof.

4. The method according to claim 3, wherein said compound is represented by the structure of formula IIIA or formula IIIB:

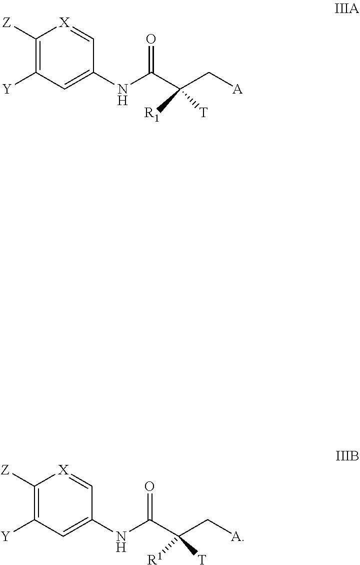

5. The method according to claim 1, wherein said compound is represented by the structure of formula IV A or formula IVB:

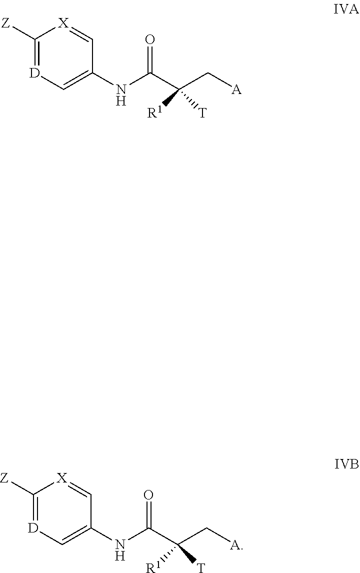

6. The method according to claim 1, wherein said compound is represented by the structure of formula V: 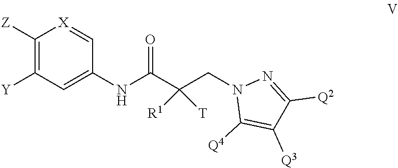
wherein
Q², Q³ and Q⁴ are each independently selected from linear or branched alkyl, haloalkyl, CF₃, aryl, F, Cl, Br, I, CN, NO₂, OR, benzyl, alkynyl, SO₂N(R)₂, NHCOOR, N(R)₂, NHCOR, CONHR, COOR, or COR; wherein said alkyl, alkynyl, and aryl are each optionally substituted with halogen, CN, or OH,
or its optical isomer, pharmaceutically acceptable salt, hydrate or any combination thereof.

7. The method according to claim 6, wherein said compound is represented by the structure of formula VA or formula VB:

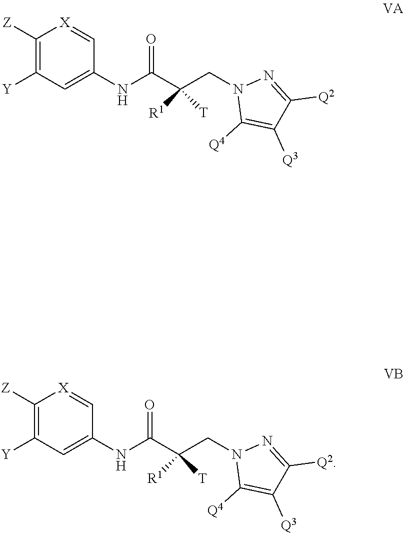

8. The method according to claim 1, wherein Q¹, Q², Q³ and Q⁴ is CN, NO₂, CF₃, F, Cl, Br, I, alkynyl, SO₂N(R)₂, NHCOOR, N(R)₂, NHCOR, COR, or phenyl, wherein said phenyl is optionally substituted with halogen, CN, or OH.

9. The method according to claim 1, wherein said compound is represented by any one of the following compounds:

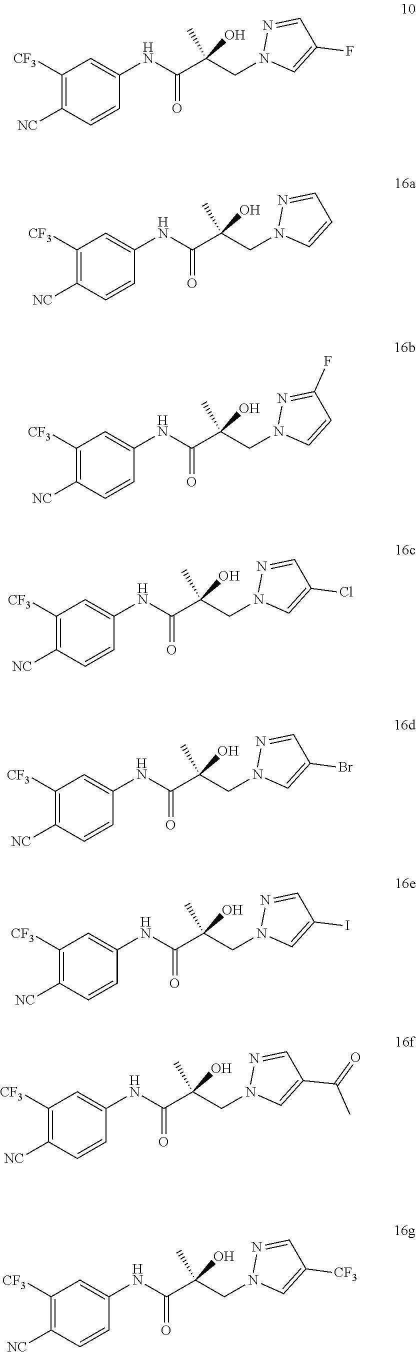

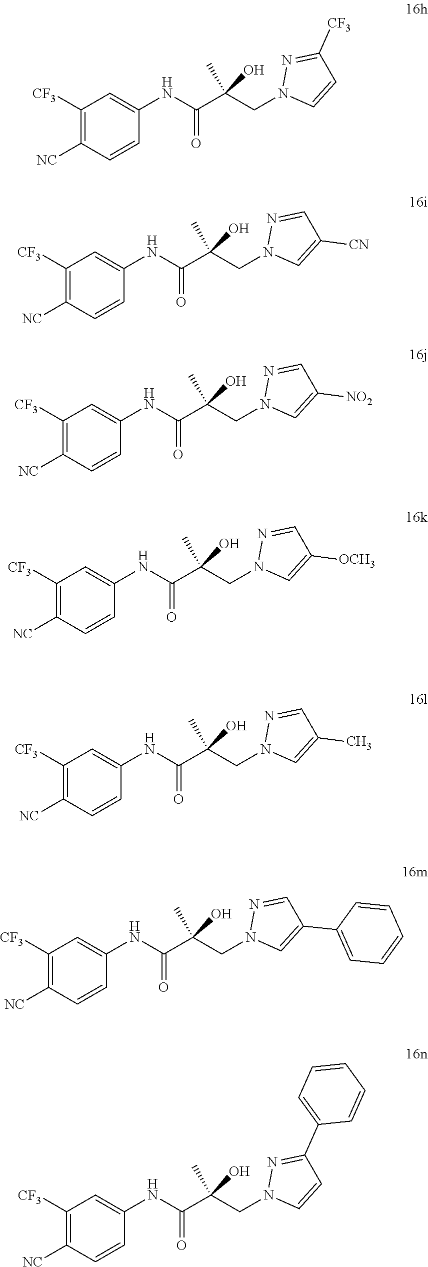

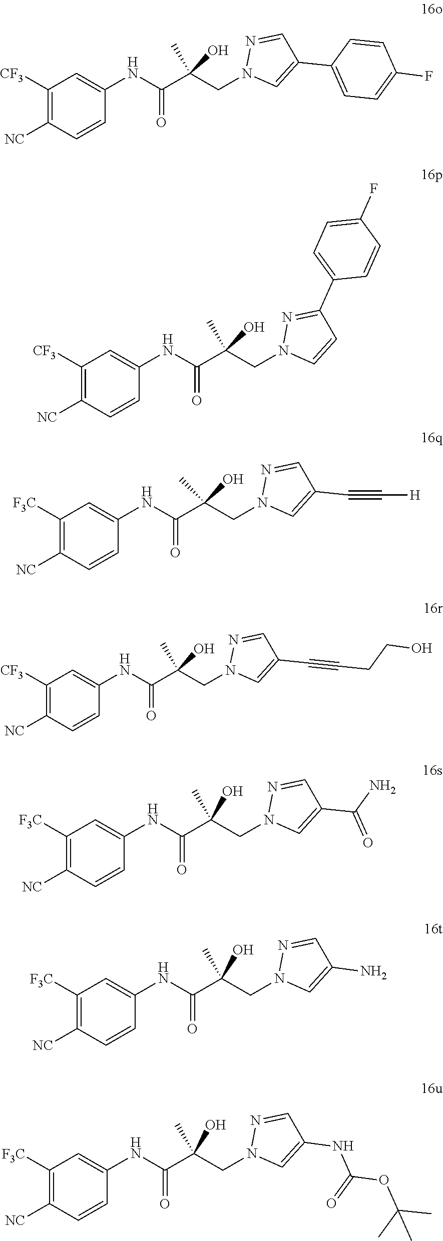

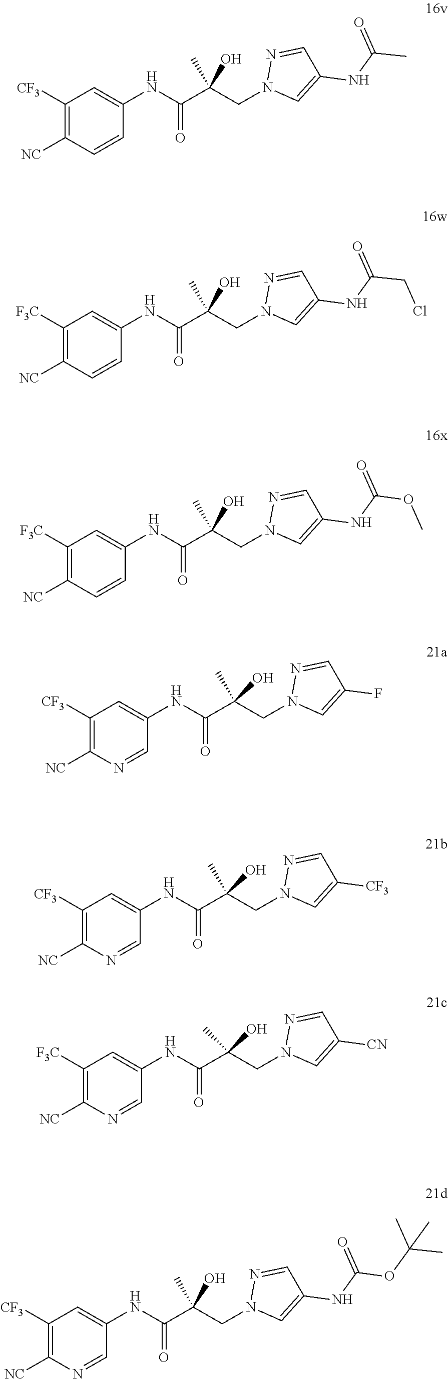

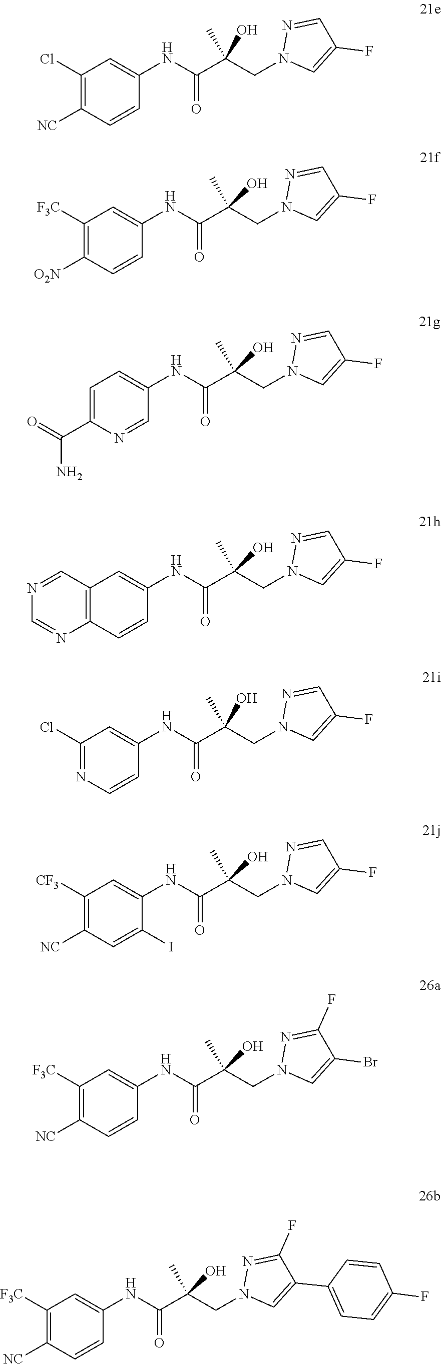

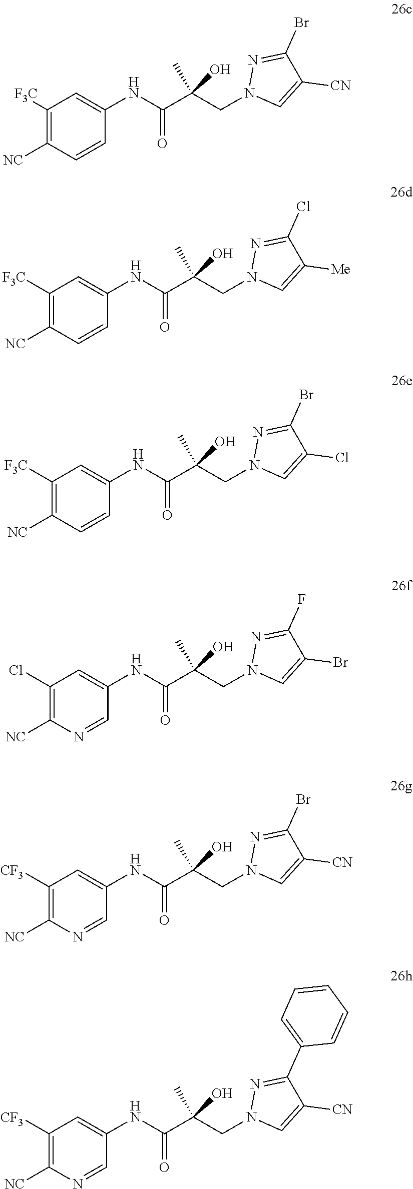 [SMILES: C[C@](O)(Cn1cc(C#N)c(Br)n1)C(=O)Nc1ccc(C#N)c(C(F)(F)F)c1 ; Cc1cn(C[C@](C)(O)C(=O)Nc2ccc(C#N)c(C(F)(F)F)c2)nc1Cl ; C[C@](O)(Cn1cc(Cl)c(Br)n1)C(=O)Nc1ccc(C#N)c(C(F)(F)F)c1 ; C[C@](O)(Cn1cc(Br)c(F)n1)C(=O)Nc1cnc(C#N)c(Cl)c1 ; C[C@](O)(Cn1cc(C#N)c(Br)n1)C(=O)Nc1cnc(C#N)c(C(F)(F)F)c1 ; C[C@](O)(Cn1cc(C#N)c(-c2ccccc2)n1)C(=O)Nc1cnc(C#N)c(C(F)(F)F)c1]

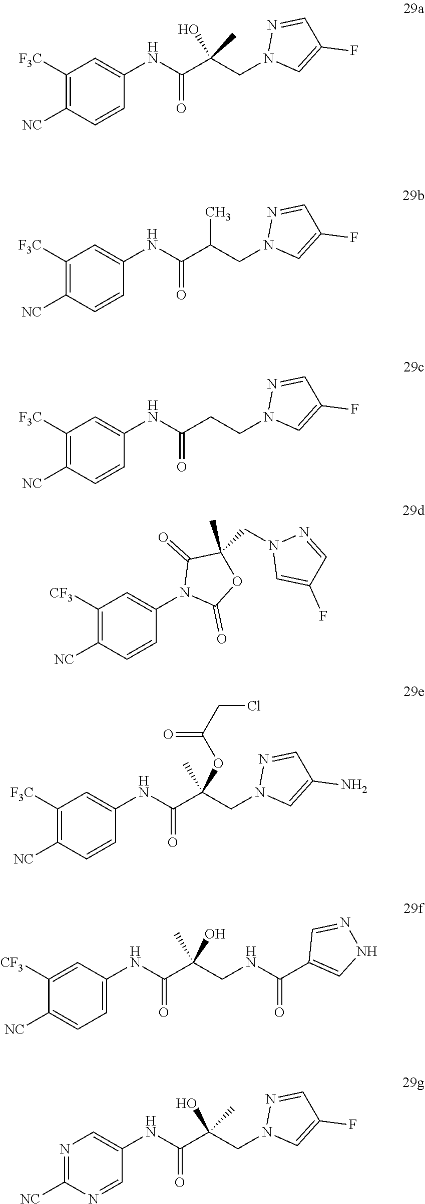

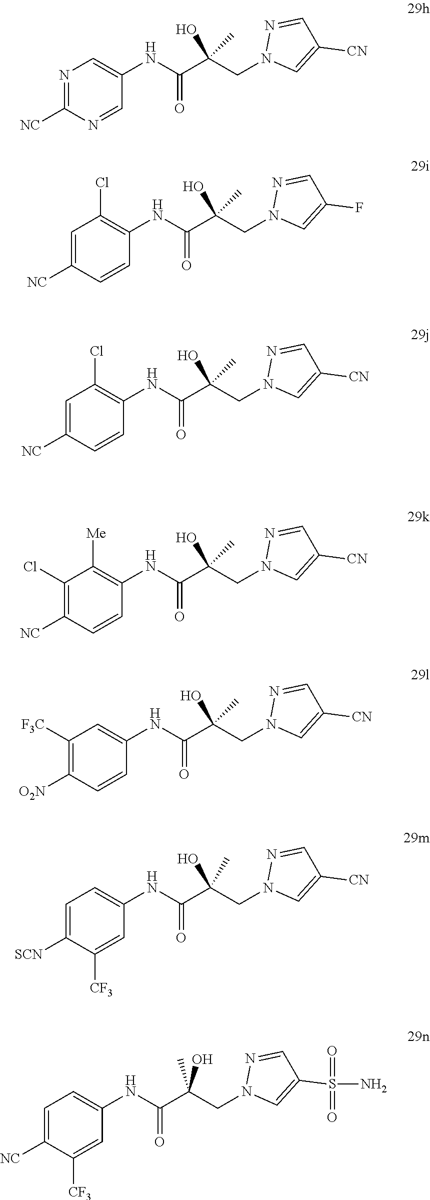

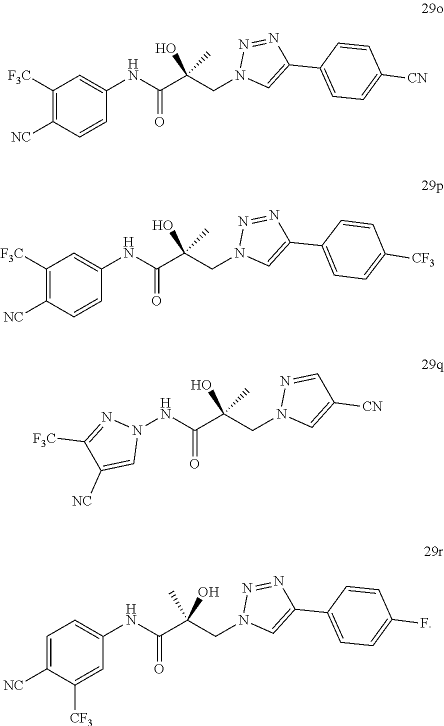 [SMILES: C[C@](O)(Cn1cc(-c2ccc(C#N)cc2)nn1)C(=O)Nc1ccc(C#N)c(C(F)(F)F)c1 ; C[C@](O)(Cn1cc(-c2ccc(C(F)(F)F)cc2)nn1)C(=O)Nc1ccc(C#N)c(C(F)(F)F)c1 ; C[C@](O)(Cn1cc(C#N)cn1)C(=O)Nn1cc(C#N)c(C(F)(F)F)n1 ; C[C@](O)(Cn1cc(-c2ccc(F)cc2)nn1)C(=O)Nc1ccc(C#N)c(C(F)(F)F)c1]

10. The method according to claim 1, wherein said compound is represented by any one of the following compounds

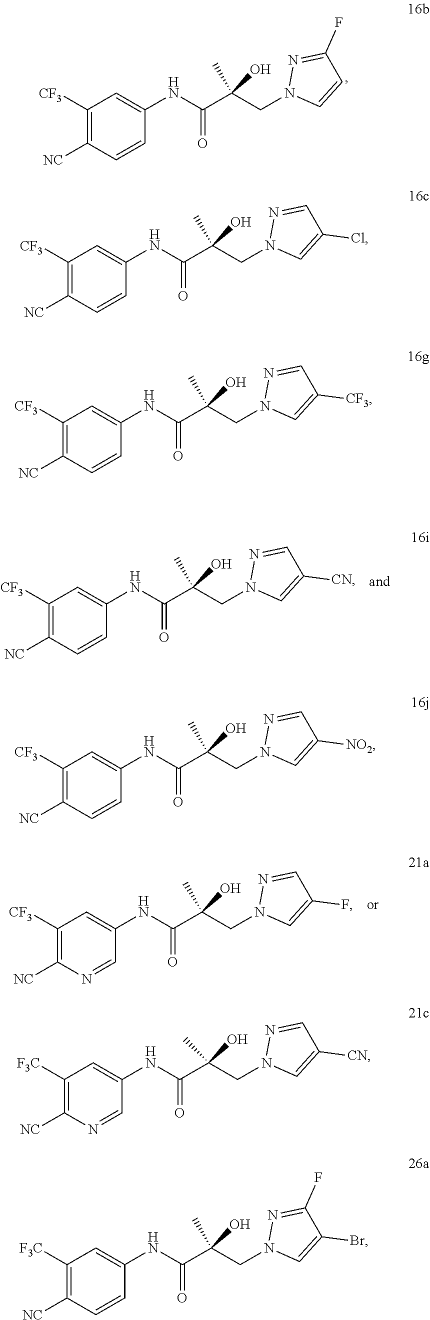

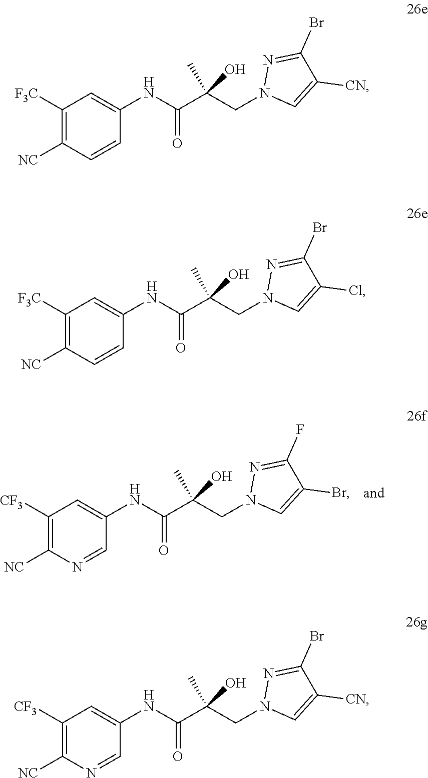 [SMILES: C[C@](O)(Cn1cc(C#N)c(Br)n1)C(=O)Nc1ccc(C#N)c(C(F)(F)F)c1 ; C[C@](O)(Cn1cc(Cl)c(Br)n1)C(=O)Nc1ccc(C#N)c(C(F)(F)F)c1 ; C[C@](O)(Cn1cc(Br)c(F)n1)C(=O)Nc1cnc(C#N)c(C(F)(F)F)c1 ; C[C@](O)(Cn1cc(C#N)c(Br)n1)C(=O)Nc1cnc(C#N)c(C(F)(F)F)c1]

11. The method according to claim 1, wherein said compound is represented by compound 26a

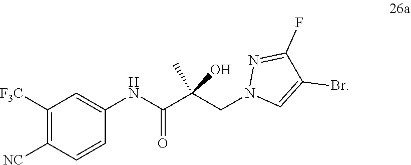 [SMILES: C[C@](O)(Cn1cc(Br)c(F)n1)C(=O)Nc1ccc(C#N)c(C(F)(F)F)c1]

12. The method according to claim 1, wherein said prostate cancer is advanced prostate cancer, refractory prostate cancer, AR overexpressing prostate cancer, castration-resistant prostate cancer, castration-sensitive prostate cancer, AR-V7 expressing prostate cancer, or d567ES expressing prostate cancer.

13. The method according to claim 12, wherein said castration-resistant prostate cancer IS AR overexpressing castration-resistant prostate cancer, F876L mutation expressing castration-resistant prostate cancer, F876L_T877A double mutation expressing castration-resistant prostate cancer, AR-V7 expressing castration-resistant prostate cancer, d567ES expressing castration-resistant prostate cancer, and/or castration-resistant prostate cancer characterized by intratumoral androgen synthesis.

14. The method according to claim 12, wherein said castration-sensitive prostate cancer is F876L mutation expressing castration-sensitive prostate cancer, F876L_T877A double mutation castration-sensitive prostate cancer, and/or castration-sensitive prostate cancer characterized by intratumoral androgen synthesis.

15. The method according to claim 12, wherein said treating of castration-sensitive prostate cancer is conducted in a non-castrate setting, or as monotherapy, or when castration-sensitive prostate cancer tumor is resistance to enzalutamide, apalutamide, and/or abiraterone.

16. The method according to claim 12, wherein the castration resistant prostate cancer (CRPC) is metastatic CRPC (mCRPC), non-metastatic CRPC (nmCRPC), or high-risk nmCRPC.

17. The method according to claim 1, further comprising administering androgen deprivation therapy (ADT).

18. The method according to claim 1, wherein the prostate cancer IS resistant to treatment with an androgen receptor antagonist.

19. The method according to claim 18, wherein the androgen receptor antagonist is at least one of darolutamide, enzalutamide, apalutamide, bicalutamide, abiraterone, EPI-001, EPI-506, AZD-3514, galeterone, ASC-J9, flutamide, hydroxyflutamide, nilutamide, cyproterone acetate, ketoconazole, or spironolactone.

20. The method according to claim 1, wherein said prostate cancer is darolutamide resistant prostate cancer, enzalutamide resistant prostate cancer, apalutamide resistant prostate cancer, or abiraterone resistant prostate cancer.

21. The method according to claim 1, wherein said prostate cancer is darolutamide resistant prostate cancer.

22. The method according to claim 1, wherein said prostate cancer is enzalutamide resistant prostate cancer.

23. The method according to claim 1, wherein said prostate cancer is apalutamide resistant prostate cancer.

24. The method according to claim 1, wherein said prostate cancer is abiraterone resistant prostate cancer.
